SEQUENCE LISTING (14 sequences)

SEQ ID NO: 1 - DNA, 3542 bp, Artificial Sequence
  H. sapiens CXCL12, transcript variant 2 (GI 76563934)
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaa

SEQ ID NO: 2 - DNA, 471 bp, Artificial sequence
  H. sapiens CXCL12, transcript variant 3 (GI 76563932)
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaaggggcgcagagaagaaaaagtggggaaaaaagaaaagataggaaaaaagaagcgacagaagaagagaaaggctgcccagaaaaggaaaaactagttatctgccacctcgagatgga

SEQ ID NO: 3 - DNA, 1937 bp, Artificial Sequence
  H. sapiens CXCL12, transcript variant 1 (GI 76563931)
gcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

SEQ ID NO: 4 - DNA, 1691 bp, Artificial Sequence
  H. sapiens CXCR4, transcript variant 2 (GI 56790928)
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 5 - DNA, 1912 bp, Artificial Sequence
  H. sapiens CXCR4, transcript variant 1 (GI 56790926)
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 6 - DNA, 3958 bp, Artificial Sequence
  H. sapiens hypoxia-inducible factor 1, alpha subunit (HIF1A), transcript variant 1 (GI 31077212)
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 7 - DNA, 3812 bp, Artificial Sequence
  H. sapiens hypoxia-inducible factor 1, alpha subunit (HIF1A), transcript variant 2 (GI 31077210)
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 8 - DNA, 5186 bp, Artificial Sequence
  H. sapiens endothelial PAS domain protein 1 (EPAS1) (GI 41327154)
gccacacgggtccggtgcccgctgcgcttccgccccagcgctcctgaggcggccgtacaatcctcggcagtgtcctgagactgtatggtcagctcagcccggcctccgactccttccgactcccagcattcgagccacttttttttttctttgaaaactcagaaaagtgactccttttccagggaaaaaggaacttgggttcccttctctccgtcctcttttcgggtctgacagcctccacccactccttccccggaccccgcctccgcgcgcaggttcctcccagtcacctttctccacccccgcccccgcacctagcccgccgcgcgccaccttccacctgactgcgcggggcgctcgggacctgcgcgcacctcggaccttcaccacccgcccgggccgcggggagcggacgagggccacagccccccacccgccagggagcccaggtgctcggcgtctgaacgtctcaaagggccacagcgacaatgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctgagccaggccttctacctgggcagcacctctgccgacgccgtcccaccagcttcactctctccgtctgtttttgcaactaggtatttctaacgccagcacactatttacaagatggacttacctggcagacttgcccaggtcaccaagcagtggcctttttctgagatgctcactttattatccctatttttaaagtacacaattgttttacctgttctgaaatgttcttaaattttgtaggatttttttcctccccaccttcaatgacttctaatttatattatccataggtttctctccctccttctccttctcacacacaactgtccatactaacaagtttggtgcatgtctgttcttctgtagggagaagctttagcttcattttactaaaaagattcctcgttattgttgttgccaaagagaaacaaaaatgattttgctttccaagcttggtttgtggcgtctccctcgcagagcccttctcgtttcttttttaaactaatcaccatattgtaaatttcagggtttttttttttttgtttaagctgactctttgctctaattttggaaaaaaagaaatgtgaagggtcaactccaacgtatgtggttatctgtgaaagttgcacagcgtggcttttcctaaactggtgtttttcccccgcatttggtggattttttattattattcaaaaacataactgagttttttaaaagaggagaaaatttatatctgggttaagtgtttatcatatatatgggtactttgtaatatctaaaaacttagaaacggaaatggaatcctgctcacaaaatcactttaagatcttttcgaagctgttaatttttcttagtgttgtggacactgcagacttgtccagtgctcccacggcctgtacggacactgtggaaggcctccctctgtcggctttttgccatctgtgatatgccataggtgtgacaatccgagcagtggagtcattcagcgggagcactgcgcgctatcccctcacattctctatgtactatgtatgtatgtattattattattgctgccaagagggtctgatggcacgttgtggggtcggggggtggggcggggaagtgctctaacttttcttaaggttttgttgctagcccttcaagtgcactgagctatgtgactcggatggtctttcacacggcacatttggacatttccagaactaccatgagatggtttagacgggaattcatgcaaatgaggggtcaaaaatggtatagtgaccccgtccacgtcctccaagctcacgaccttggagccccgtggagctggactgaggaggaggctgcacagcgggagagcagctggtccagaccagccctgcagcccccactcagccggcagccagatggccccgcaaggcctccagggatggcccctagccacaggccctggctgaggtctctgggtcggtcagtgacatgtaggtaggaagcactgaaaatagtgttcccagagcactttgcaactccctgggtaagagggacgacacctctggtttttcaataccaattacatggaacttttctgtaatgggtacaatgaagaagtttctaaaaacacacacaaagcacattgggccaactatttagtaagcccggatagacttattgccaaaaacaaaaaatagctttcaaaagaaatttaagttctatgagaaattccttagtcatggtgttgcgtaaatcatattttagctgcacggcattaccccacacagggtggcagaacttgaagggttactgacgtgtaaatgctggtatttgatttcctgtgtgtgttgccctggcattaagggcattttacccttgcagttttactaaaacactgaaaaatattccaagcttcatattaaccctacctgtcaacgtaacgatttcatgaacgttattatattgtcgaattcctactgacaacattataactgtatgggagcttaactttataaggaaatgtattttgacactggtatcttattaaagtattctgatcctaaaaaaaaaaaaaaaaaaaaaaaaaaaaaaaaaaaaaaaaaaaaaaaaa

SEQ ID NO: 9 - DNA, 5228 bp, Artificial Sequence
  H. sapiens v-ets erythroblastosis virus E26 oncogene homolog 1 (avian) (ETS1) (GI 41393580)
cgggcgagggccgggcaggaggagcgggcgcggcgcgggcgaggctgggacccgagcgcgctcacttcgccgcaaagtgccaacttcccctggagtgccgggcgcgcaccgtccgggcgcgggggaaagaaaggcagcgggaatttgagatttttgggaagaaagtcggatttcccccgtccccttccccctgttactaatcctcattaaaaagaaaaacaacagtaactgcaaacttgctaccatcccgtacgtcccccactcctggcaccatgaaggcggccgtcgatctcaagccgactctcaccatcatcaagacggaaaaagtcgatctggagcttttcccctccccggatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtgatggcactgaaggggctggggaaaccctgctgagaccttccaaggacagccgtgttggttggactctgaattttgaattgttattctattttttattttccagaactcattttttaccttcaggggtgggagctaagtcagttgcagctgtaatcaattgtgcgcagttgggaaaggaaagccaggacttgtggggtgggtgggaccagaaattcttgagcaaattttcaggagagggagaagggccttctcagaagcttgaaggctctggcttaacagagaaagagactaatgtgtccaatcatttttaaaaatcatccatgaaaaagtgtcttgagttgtggacccattagcaagtgacattgtcacatcagaactcatgaaactgatgtaaggcaattaatttgcttctgtttttaggtctgggagggcaaaaaagaggtgggtgggatgaaacatgttttggggggggatgcactgaaaatctgagaactatttacctatcactctagttttgaagcaaagatggacttcagtggggaggggccaaaaccgttgttgtgttaaaatttattttattaaattttgtgccagtattttttttcttaaaaatcgtcttaagctctaaggtggtctcagtattgcaatatcatgtaagtttgtttttatttgccggctgaggattctgtcacaatgaaagaaaactgtttatatagaccccattggaaaagcaaaacgctctcactgagatcagggatcccaaattcatgggacttatataagaaggacaattaatgctgatttgggtacaggggaattatgtgtgtgaatgtcatctacaattaaaaaaaattagcacatccctttacttacttgttatcagtggattctcggggtttggacttaatgttgagctaagaagcattaagtctttgaactgaatgtattttgcatccctggttttggacgacagtaaacgtaggagcactgttgaagtcctggaagggagatcgaaggaggaagattgacttggttctttcttagtcctatatctgtagcatagatgacttggaataaaagctgtatgcatgggcattacccctcaggtcctaagaaataagtcctgaatgcatgtcgttccaaactaacactctgtaatttttcttttatgtcttattttccaagagtcctccattttttgcaccccctcaccgccaactctgttattcagtagagagaagtgtacggctttctgattggtgagtgaaaaagtaacttgagacacgacctaagttgaagagtttagacttgctgagttttagaagtgatggaaattaagagagcatttcaataaaatgtgacttggctgtctttggaagagaagtgcaaggctttcctttgaagaatttaaattagtccggtaggatgtcaggtgagactgtgtatgcaaaatgaatggcacaggtgatgccagggcctcttgcttgggtctgatgtcttggcacagggtaagtgaaggttaattccagaagagaggaatgacttgaaggcaaaggaaactaaggaaggaggttcagtgaggaaaataaggttgtccatgagatttgaatagatttttagttcccccaaggtttaaatacaaacatagtcaagcaaggtagtcatctttctgctggttgtgagggggaatctgaaaatggagttttagaggaaaagtcaacatctaactagtgaggaaaagtgcctaatacaattagaatctccctcactctatagttgcccagttgaaaggataaggaggaggggtggcttttatggacttccatgagagaaggaaagaaatatttcaggtaagcttctcagggctggccctttttgggatttggatgagaaattggaagtactaactactttctagcatatctttaagaaaattgattgttatttactcccagatcctcttgcagacccagaattatcaggaacatagctctgtgattcatgagtgtccccatactgatgaattggagcatccatatggaaagcaaaggcagaattatcccagctgtattattttgatcttttggatgcaggtgccttaatgaagctctcaaaatattttaggagctgctcagggagtgttgggtggaactgtttggactacattgttttctcttagattatgtgatttttgttgggcactggcaaaaggtgtgtgtgtgaatgtgtgcatgtgtgtgaatgttgtgtgtgtgtgtgtgtgtgtgtgtgtgtgtgtgtgtgtttgcagacatgcaaaactgcagctgaaataataccttagatttctaggtaagtctttccacatttcaataatgggtaagagtagaaccagggccgggtatcaattattgcttgctgtttgcaaccaggcataaaatcactttctcaaatcatccaccgttcctattaaatttatgccggaaactctccttctgtgagtataactcctgcagttcctatagcagataagatataagaaagtgcctcctagtgctcctccgcccgcttgtttgctaaaattccctttctctctaagtccaccattttcaagatttgtagatagtgtattagttaagacagctttgtcgatctggccagatgttttttctcctttgtccaaaggccagagaccatcccaggaagagtggtgggtggtttatacactggaaatgttgcgtttatgctttttaaaaacacacgttaacttcagaggaaggatgggcaaatctggtctagctgggtgaaacccttattttcccagagatgccttaacctttgttggttttggctttagggttcagagtcacttttgttcccttctccattctggagagggacttcccctacatagagccctgatttttgtggctgtggggattggaggtagcattcaaagatcagatgtgcttttcctcactttggagatgaacactctgggttttacagcattaacctgcctaaccttcatggtgagaaatacaccatctctcttctagtcatgctgtgcatgccgcttactctgttggggtctatataaatttgttgaactcttacctacattccaaagaagtttcaaggaaccataaatatatgtatacatatacatatataaaatatatatattaaaataaaattatcaggaatactgcctcagttattgaacttttttttttaagaatactttttttttaagctgagaagtatagggatgaaaaagatgttatattgtgtttgactattttccaacttgtattttcatataatttatattttttaaaagctgaaaatttagaagcaagatgaaaaaaaggaaaagcaggtgctttttaaaaatcagaactgaggtagcttagagatgtagcgatgtaagtgtcgatgtttttttaaaaaaaaatgcaaaaaaattcttatggcggagttttttgtttgtttattttagtagctgatgctggcacatcattttgctggagagttttttatatactgtagcctgatttcatattgtattttaaactgtgtgaaattaaaaacaaagaatttcattcataaaaaaaaaaaaaaaaaaaaaaaaaaaaaaaaaaaaaaaaaa

SEQ ID NO: 10 - DNA, 4104 bp, Artificial Sequence
  H. sapiens nuclear factor of kappa light polypeptide gene enhancer in B-cells 1 (p105) (NF-kappaB1) (GI 34577121)
gtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctcaaaaaaaaaaaaaaaaaa

SEQ ID NO: 11 - DNA, 3001 bp, Artificial Sequence
  H. sapiens nuclear factor of kappa light polypeptide gene enhancer in B-cells 2 (p49/p100) (NF-kappaB2) (GI 19923222)
actttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcacctcccagcccatccatgatagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgcggtgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcacgcagccccgagccctactcgactactgcgtcaccgcggacgccgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccttggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctcgcacgggcacccccagcctcaggtgactgacctgctgcctgcccccagcccccttcccggaccccctgtacagcgtccccacctatttcaaatcttatttaacaccccacacccacccctcagttgggacaaataaaggattctcatgggaaggggaggaccccgaattcct

SEQ ID NO: 12 - DNA, 3054 bp, Artificial Sequence
  H. sapiens nuclear factor of kappa light polypeptide gene enhancer in B-cells 2 (p49/p100) (NF-kappaB2), transcript variant 2 (GI 117320526)
ggtattttcgggactttcctaagctgctctaactttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttaaaaaaaaaaaaa

SEQ ID NO: 13 - DNA, 1760 bp, Artificial Sequence
  H. sapiens v-rel reticuloendotheliosis viral oncogene homolog A, nuclear factor of kappa light polypeptide gene enhancer in B-cells 3, p65 (avian) (RELA) (GI 46430498)
ggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtccttcctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgg

SEQ ID NO: 14 - DNA, 2287 bp, Artificial Sequence
  H. sapiens v-rel reticuloendotheliosis viral oncogene homolog B, nuclear factor of kappa light polypeptide gene enhancer in B-cells 3 (avian) (RELB) (GI 35493877)
cgcgccccgcgcagccccgggcgccgcgcgtcctgcccggcctgcggccccagcccttgcgccgctcgtccgacccgcgatcgtccaccagaccgtgcctcccggccgcccggccggcccgcgtgcatgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtagccccgcgatgccagaggaggggcactgggtggggagggaggtggaggagccgtgcaatcccaaccaggatgtctagcacccccatccccttggcccttcctcatgcttctgaagtggacatattcagccttggcgagaagctccgttgcacgggtttccccttgagcccattttacagatgaggaaactgagtccggagaggaaaagggacatggctcccgtgcactagcttgttacagctgcctctgtccccacatgtgggggcaccttctccagtaggattcggaaaagattgtacatatgggaggagggggcagattcctggccctccctccccagacttgaaggtggggggtaggttggttgttcagagtcttcccaataaagatgagtttttgagcctcaaaaaaaaaaaaaaaaa

(19) United States
(12) Patent Application Publication
(10) Pub. No.: US 2010/0298214 A1
(43) Pub. Date: Nov. 25, 2010

(54) TREATMENT OF AUTOIMMUNE DISEASE

(75) Inventors: Qibin Leng, Beijing (CN); Jianzhu Chen, Lexington, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 12/672,436

(22) PCT Filed: Aug. 29, 2008
(86) PCT No.: PCT/US08/74910

§ 371 Date: Jun. 16, 2010

Related U.S. Application Data
(60) Provisional application No. 60/969,362, filed on Aug. 31, 2007.

(51) Int. Cl. A61K 38/16; C12Q 1/04; A61K 38/22; C40B 40/00; A61K 31/7105; A61K 31/711; A61K 38/02; A61P 37/02; A61P 3/10

(52) U.S. Cl. 514/6.9; 435/34; 514/9.7; 506/13; 514/44 R; 514/1.1

(57) ABSTRACT

The present invention provides compositions, systems, and methods for identifying a patient suffering from and/or susceptible to autoimmune disease who might be likely to respond to treatment with CXCL12 and/or CXCR4 antagonists. The present invention provides novel CXCL12 and/or CXCR4 antagonists, methods of identifying novel CXCL12 and/or CXCR4 antagonists, and methods involving the use of these in the treatment of autoimmune disease.

### GOVERNMENT SUPPORT

The United States Government has provided grant support utilized in the development of the present invention. In particular, the National Institutes of Health (contract numbers A150631 and A169208) has supported development of this invention. The United States Government has certain rights in the invention.

### BACKGROUND OF THE INVENTION

Autoimmunity is the failure of an organism to recognize its own constituent parts (down to the sub-molecular levels) as “self,” which typically results in an immune response against its own cells and tissues. Any disease that results from such an aberrant immune response is termed an autoimmune disease. Prominent examples include diabetes mellitus type 1, systemic lupus erythematosus (SLE), Sjögren's syndrome, multiple sclerosis, Hashimoto's thyroiditis, Graves' disease, rheumatoid arthritis (RA), and psoriasis. Symptoms of an autoimmune disease can vary widely and depend on the specific disease. A group of very nonspecific symptoms often accompany autoimmune diseases, and may include dizziness, fatigue, general malaise, and low-grade fever.

There is a need in the art for systems, methods, and/or compositions for inhibiting and/or delaying the onset of autoimmune disorders, such as diabetes. There is a need in the art for methods for identifying patients who are likely to respond to a particular treatment, as the ability to identify such patients would minimize side effects and open new avenues for “personalized” therapy.

### SUMMARY OF THE INVENTION

The present invention provides compositions, systems, and methods for identifying a patient who might be likely to respond to treatment with CXCL12 and/or CXCR4 antagonists, as described herein. The present invention encompasses the recognition that some autoimmune disorders (e.g., diabetes) are associated with elevated levels and/or activity of CXCL12 and/or CXCR4 in certain tissues and/or cells (e.g., bone marrow, blood, etc.). The present invention encompasses the recognition that some autoimmune disorders (e.g., diabetes) arc associated with elevated levels of naïve T cells and/or stem cells in certain tissues and/or cells (e.g., bone marrow). The present invention encompasses the recognition that identification of patients suffering from or susceptible to an autoimmune disorder that is associated with elevated levels of CXCL12 is desirable because it allows for identification of patients who might be likely to respond to particular therapies (e.g., CXCL12 and/or CXCR4 antagonists). In some embodiments, the present invention encompasses the recognition that CXCL12 and/or CXCR4 antagonists (e.g., AMD3100) may be utilized for treatment and/or prophylaxis of autoimmune disorders (e.g., type I diabetes) in humans.

In general, a CXCL12 and/or CXCR4 antagonist is any substance that negatively affects the ability of CXCL12 to bind to CXCR4 (i.e., “the CXCL12-CXCR4 interaction”). A CXCL12 and/or CXCR4 antagonist in accordance with the invention may be one which exerts its modulatory effect upstream, downstream, and/or directly on CXCL12 and/or CXCR4. According to the present invention, CXCL12 and/or CXCR4 antagonists may be small molecules, proteins (e.g., peptides, antibodies, etc.), nucleic acids (e.g., antisense oligonucleotides, ribozymes, siRNAs, etc.), lipids, carbohydrates, viruses, etc.

The present invention provides novel CXCL12 and/or CXCR4 antagonists and method of identifying novel CXCL12 and/or CXCR4 antagonists. In some embodiments, the present invention provides in vitro methods for screening for CXCL12 and/or CXCR4 antagonists. For example, in some embodiments, a method generally comprises steps of: (1) providing a test substance (e.g., CXCL12 and/or CXCR4 protein and/or the CXCL12 and/or CXCR4 gene); (2) providing a candidate substance; and (3) measuring and/or detecting an influence of the candidate substance on the test substance. For example, in some embodiments, binding assays involve exposing CXCL12 and CXCR4 proteins (including homologs, portions, variants, mutants, and/or derivatives thereof) to a candidate substance and detecting binding between CXCL12 and CXCR4 in the presence of the candidate substance.

In some embodiments, the present invention provides in cyto methods for screening for CXCL12 and/or CXCR4 antagonists. For example, such methods may involve contacting a candidate substance with a cell. The cell can then be assayed for various parameters associated with CXCL12 and/or CXCR4 activity. For example, parameters associated with CXCL12 and/or CXCR4 activity include, but are not limited to, the ability of CXCL12 to bind to CXCR4.

In some embodiments, the present invention provides in vivo methods for screening for CXCL12 and/or CXCR4 antagonists. In vivo assays utilize various animal models, including transgenic animals that have been engineered to have specific defects and/or carry markers that can be used to measure the ability of a candidate substance to reach and/or affect different cells within an organism. In such assays, one or more candidate substances are administered to an animal, and the ability of a candidate substance(s) to alter one or more characteristics, as compared to a similar animal not treated with the candidate substance(s), identifies a CXCL12 and/or CXCR4 antagonist. Such characteristics may be any of those discussed herein with regard to symptoms associated with an autoimmune disorder (e.g., diabetes) and/or accumulation of T cells and/or stem cells in bone marrow. To give but one example, methods of identifying novel CXCL12 and/or CXCR4 antagonists useful for treatment of diabetes may comprise steps of (1) providing a mouse exhibiting symptoms of diabetes (e.g., NOD mouse), (2) administering a candidate substance to the mouse, (3) assaying for increased mobilization of naïve T cells and/or stem cells from bone marrow to peripheral lymphoid organs (e.g., by measuring changes in percentage of cells and/or number of cells in bone marrow with or without treatment).

Compositions, systems, and methods described herein can be useful for identifying patients suffering from or susceptible to an autoimmune disorder (e.g., diabetes) that is associated with elevated levels of CXCL12 and/or CXCR4 in a particular tissue (e.g., bone marrow, blood, etc.), and/or treatment and/or diagnosis of such an autoimmune disorder. Compositions, systems, and methods described herein can be useful for identifying patients suffering from or susceptible to an autoimmune disorder (e.g., diabetes) that is associated with elevated levels of naïve T cells and/or stem cells in a particular tissue (e.g., bone marrow), and/or treatment and/or diagnosis of such an autoimmune disorder.

Thus, in some embodiments, the present invention provides methods comprising steps of: (1) providing a subject suffering from and/or susceptible to an autoimmune disorder, such as diabetes, (2) assaying levels of CXCL12 in a particular test tissue (e.g., bone marrow and/or blood), (3) identifying patients with elevated levels of CXCL12 in the test tissue, and (4) administering to these patients a therapeutic amount of CXCL12 and/or CXCR4 antagonist that is sufficient to treat, alleviate, ameliorate, relieve, delay onset of, inhibit progression of, reduce severity of, and/or reduce incidence of one or more symptoms or features of an autoimmune disorder.

Alternatively or additionally, levels of CXCL12 in a particular test tissue (e.g., bone marrow) may be assayed indirectly by measuring levels of CXCL12 in a test subject's blood. For example, the present invention encompasses the recognition that assaying levels of CXCL12 and/or CXCR4 in blood can correlate with levels of CXCL12 and/or CXCR4 in bone marrow. Alternatively or additionally, elevated levels in blood could lead accumulation of naïve T cells and/or stem cells in blood, thereby reducing their level in other lymphoid organs (e.g., spleen and lymph nodes).

In some embodiments, levels of CXCL12 in a particular test tissue may be assayed in vitro using cell migration assays. Cell migration assays are well-known and can be designed and carried out in any way determined by one of ordinary skill in the art. For example, cells expressing CXCR4 on their surfaces may be exposed to a sample (e.g., from a subject's bone marrow, blood, etc.), and the responsiveness of the cells to the sample (e.g., rate of migration toward the sample, distance migrated, etc.) can serve as a measure of CXCL12 levels in the sample.

The present invention encompasses the recognition that patients exhibiting elevated levels of CXCL12 in particular tissues (e.g., bone marrow) might be likely to respond to therapies involving CXCL12 and/or CXCR4 antagonists.

The present invention provides methods of treating and/or diagnosing a patient who is suffering from and/or is susceptible to an autoimmune disorder (e.g., diabetes). The present invention encompasses the recognition that some autoimmune disorders (e.g., diabetes) are associated with elevated levels of CXCL12 in particular tissues (e.g., bone marrow). The present invention encompasses the recognition that autoimmune disorders that are associated with elevated levels of CXCL12 in particular tissues may be treated with CXCL12 and/or CXCR4 antagonists.

Compositions in accordance with the present invention may be administered using any amount and any route of administration effective for treatment, including, but not limited to, oral, systemic intravenous injection, regional administration via blood and/or lymph supply, and/or direct administration to an affected site.

Pharmaceutical compositions in accordance with the present invention may be administered either alone or in combination with one or more other therapeutic agents.

In some embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of an autoimmune disorder. For example, compositions in accordance with the invention may be administered in combination with traditional diabetes therapies including, but not limited to, insulin administration. To give another example, compositions in accordance with the invention may be administered in combination with soluble TNF receptor, anti-TNFα receptor, analgesics, non-steroidal anti-inflammatory agents (NSAIDs), and/or other agents may be useful for treatment of rheumatoid arthritis.

The invention provides a variety of kits comprising one or more composition(s) in accordance with the invention. For example, a kit may include materials useful for identifying and/or screening for patients who may be likely to respond to treatment with CXCL12 and/or CXCR4 antagonists. Such a kit may include, for example, (i) equipment suitable for obtaining a bone marrow and/or blood sample from a subject; (ii) an antibody that recognizes CXCL12 in western blotting and/or ELISA assays; (iii) CXCL12 protein that may serve as a positive control for western blotting and/or ELISA assays; (iv) a reference bone marrow and/or blood sample (e.g., samples from non-diabetic individuals).

This application refers to various issued patents, published patent applications, journal articles, and other publications, all of which are incorporated herein by reference in their entirety.

### BRIEF DESCRIPTION OF THE DRAWING

FIG. 1: Accumulation of naïve T cells in the bone marrow of NOD mice. (A) Frequency of CD4 T cells in the bone marrow of BALB/c, prediabetic, and diabetic NOD mice. Cells from bone marrow (BM), lymph nodes (LN; including cervical, mediastinal, auxiliary, brachial, mesenteric, and inguinal nodes), and spleen (SP) were stained for CD4, CD8, TCRβ, and PI. CD4 versus TCRβ profiles are shown for live cells (PI-negative). The numbers indicate the percentages of CD4⁺TCR⁺ cells in the gated regions. p<0.001 comparing percentages of CD4 T cells in bone marrow between BALB/c and NOD mice. (B) The average number of CD4 (open bar) and CD8 (solid bar) T cells in the BM, LN, and SP of BALB/c (n=22), prediabetic (n=19), and diabetic (n=16) NOD mice. p<0.01 comparing CD4 or CD8 T cells in bone marrow between BALB/c and prediabetic or diabetic NOD mice. (C) Gradual increase of percentages of CD4 T cells with age (16-27 weeks) in the bone marrow of diabetic NOD mice. CD4 T cells were assayed the same way as in (A). (D) Inverse correlation between percentage of CD4 T cells in the bone marrow and CD4 T cell number in the spleen of the same diabetic NOD mice. r² value is =0.69. Data presented in (C) and (D) are from the same group of mice. One dot represents one mouse. (E) Comparison of percentages of CD4 T cells in the bone marrow of 4-5 week old BLAB/c (n=6) and NOD (n=8) mice. p<0.001. (F) Comparison of percentages of CD4 T cells in the bone marrow of 6-9 week old BALB/c (n=5), EA16 (n=3), and NOD (n=8) mice. p<0.001 comparing BALB/c to NOD (or EA16) mice. (G) Phenotype of bone marrow T cells. Bone marrow cells from BALB/c, prediabetic, and diabetic NOD mice were stained for TCR; CD4; CD25 plus CD45RB or CD62L; or TCR, CD4, CD44 and CD45RB. Representative plots from at least four independent experiments are shown, gating on CD4′TCR′ cells. The numbers indicate the percentages of cells in the gated regions.

FIG. 2: T cell accumulation in the bone marrow of NOD mice is due to homing, not proliferation. (A) Comparison of steady-state level of CD4 T cell proliferation in the bone marrow of age-matched BALB/c and prediabetic NOD mice (15-16 weeks of age). Bone marrow cells were stained for TCR, CD4, CD44, and Ki67. Representative Ki67 versus CD44 expression profiles are shown for TCR⁺CD4⁺cells. The numbers are percentage of Ki67⁺ cells. (B) Comparison of T cell homing to the bone marrow and lymph nodes in BALB/c and NOD mice. CFSE-labeled T cells from BALB/c or NOD mice were injected intravenously into both BALBic and NOD recipients (12-16 week old) and analyzed 2 hours following the transfer. Homing index is calculated by dividing percentage of CFSE-positive donor CD4 T cells in the bone marrow or lymph nodes by that in the spleen of the same recipient. Mean ±SD of homing index of CD4 T cells in at least four mice per group is shown. *p<0.01.

FIG. 3: T cells preferentially home to the bone marrow of NOD mice. T cells from NOD mice were labeled with CSFE and injected intravenously into prediabetic NOD mice and BALB/c mice and analyzed 48 hours later. Homing index is calculated by dividing the percentage of CFSE-positive donor CD4 T cell numbers in bone marrow or lymph nodes by that in the spleen of the same recipient. Mean±SD of homing index of CD4 T cells in at least four mice per group is shown. *p<0.01.

FIG. 4: Elevated CXCL12 expression correlates with T cell accumulation in the bone marrow of NOD mice. (A) Analysis of chemokine expression in the bone marrow. RNA was isolated from bone marrows of BALB/c (n=5) and prediabetic NOD (n=6) mice, pooled, labeled, and used as a probe to hybridize with GEArray chemokine array filters. Representative chemiluminescent images from the hybridization are shown. 1, CCL19; 2, CXCL12. (B) Quantitation of CXCL12 and CCL19 transcripts by RT-PCR. The same RNA as in A was used in real-time RT-PCR analysis for CXCL12, CCL19, and GAPDH. The relative transcript levels of CXCL12 and CCL19 to GAPDH are shown. (C) Comparison of CXCL12 transcript levels in the bone marrow between 4-5 week-old NOD mice (n=8) and age-matched BALB/c mice (n=6). (D) Comparison of CXCL12 transcript levels in the bone marrow among 6-9 week old BALB/c (n=5), EA16 (n=3), and NOD (n=8) mice. (E)

CXCR4 expression by CD4 T cells in different organs of BALB/c and prediabetic NOD mice. Cells from BM, LN and SP of BALB/c and prediabetic NOD mice were stained for TCR, CD4, CD45RB, and CXCR4. Representative CXCR4 versus CD45RB profiles are shown for TCR⁺CD4⁺ cells. The numbers indicate percentages of cells in the gated areas. *p<0.05

FIG. 5: AMD3100 inhibits naïve T cell accumulation in the bone marrow of NOD mice. Prediabetic NOD mice (15-16 weeks of age) were treated with PBS or AMD3100 (AMD) daily for 8 days. Two hours after the last AMD3100 injection, mice were analyzed by flow cytometry. (A) Comparison of percentage of CD4 and CD8 T cells (mean±SD) in the bone marrow of AMD3100 (n=4) and PBS (n=6) treated NOD mice. *p<0.05. (B) Comparison of CD44 versus CD45RB profiles of TCR⁺CD4⁺cells from AMD3100 (n=4) and PBS (n=6) treated NOD mice. The numbers indicate percentages of cells in the gated areas. The percentages (mean±SE) of CD45RB^{hi} CD44^{lo} naïve T cells are 47.3±10.9 for PBS-treated mice and 24.8±3.2 for AMD3100-treated mice (p<0.01).

FIG. 6: AMD3100 treatment does not affect NKT cell distribution. Prediabetic NOD mice were given either PBS or AMD3100 for 8 days. NKT cells were assayed by staining with anti-TCRβ and CD1d loaded with PBS57 ligand. The numbers indicate the percentages of NKT cells in various organs.

FIG. 7: The elevated CXCL12 expression promotes recruitment/retention of Treg and hematopoietic stem cells in the bone marrow. (A) Comparison of the percentages and numbers of Foxp3⁺CD4⁺ Tregs in the bone marrow between age-matched BALB/c (n=7) and prediabetic NOD (n=20) mice (15 weeks of age). Bone marrow cells were assayed for TCR, CD4 and Foxp3. Tregs are identified as TCR⁺Foxp3⁺CD4⁺ cells. The percentage value of Foxp3⁻ cells is expressed as percent of CD4⁺ T cells. (B) Effect of CXCR4 deletion on Treg distribution in the spleen and bone marrow. Cells from spleen and bone marrow of Cxcr4^{f/f} Lck-Cre (KO) mice and littermate Cxcr^{+/f} Lck-Cre (WT) (8 weeks of age) were assayed for TCR, CD4, CD25 and Foxp3. The numbers of TCR⁺CD4⁺CD25⁺Foxp3⁺ cells are compared in the spleen and bone marrow from 3 mice per group. (C) Comparison of numbers of hematopoietic stem cells (Lin⁻Sca1⁺c-Kit⁺, LSK) among age-matched BALB/c (n=5), prediabetic NOD (n=5) and AMD3100-treated (8 days) prediabetic NOD mice (n=4). *p<0.05.

FIG. 8: AMD3100 treatment inhibits leukocyte infiltration and development of diabetes. (A) Immunohistological staining of pancreatic sections of NOD mice. The pancreas of prediabetic, diabetic NOD mice, or prediabetic NOD mice that have been given AMD3100 for 8 days were fixed and embedded. Parallel tissue sections were stained with haematoxylin and eosin (H&E, top panel), anti-glucagon (middle panel), or anti-insulin (bottom panel) antibodies. Note lymphocyte infiltration in the islets of prediabetic NOD mouse without AMD3100 treatment. (B) Percentage of peri-insulitis and insulitis in pancreatic sections of 15 week-old prediabetic NOD mice that were given daily with PBS (n=8) or AMD3100 (n=6) for 8 days. *p<0.01. (C) and (D) Comparison of diabetes incidence in NOD mice that were given AMD3100 or PBS for 3 weeks (C) or 14 weeks (D), starting at 15-16 weeks of age. Number of mice in each group (n) is shown. Mice are scored as diabetic when glucose level in the urine reaches 500 mg/dl. p<0.01.

FIG. 9: CFA prevents development of diabetes in NOD mice. NOD mice (15 weeks of age) were given a single CFA injection subcutaneously. Mice were monitored for diabetes by measuring urine glucose level. Mice were scored diabetic when glucose level reaches 500 mg/dl. Number of mice in each group (n) is shown.

FIG. 10: Elevated CXCL12 expression likely contributes to diabetes in NOD mice through multiple mechanisms. (A) and (B) Inhibition of CXCL12 expression and T cell accumulation in the bone marrow of NOD mice by CFA. Prediabetic NOD mice were given a single injection of CFA. Percentages of CD4 T cells and CXCL12 expression were measured 2 weeks later. (A) The average percentage of CD4 T cells in the BM of age-matched CFA-treated (n=5) and untreated (n=8) NOD mice. (B) Comparison of CXCL12 transcript level in the BM of CFA-treated (n=5) and untreated (n=8) NOD mice. The error bar shows the one standard deviation. (C) Comparison of percentages of hematopoietic stem cells (Lin⁻Sca1⁺c-Kit⁺, LSK) between age-matched BALB/c mice (n=5) and prediabetic NOD mice (n=8). *p<0.05. (D) Comparison of percentages of hematopoietic stem cells in prediabetic NOD mice following daily injection of AMD3100 or PBS for 8 days. *p=0.06. (E) Comparison of the percentages and numbers of Foxp3⁺CD4⁺ Tregs in the bone marrow between age-matched BALB/c and prediabetic NOD mice (15 weeks of age). *p<0.05. (F) Effect of CXCR4 deletion on Treg distribution in the spleen and bone marrow. Cells from spleen and bone marrow of Cxer4^{f/f} Lck-cre (KO) mice and littermate Cxcr4^{+/f} Lck-cre (WT) mice (12 weeks of age) were assayed for TCR, CD4, CD25, and Foxp3. The numbers of TCR⁺CD4⁺CD25⁺Foxp3⁺ cells are compared in the spleen and bone marrow from 3 mice per group. P values are shown. (G) and (H) Comparison of the percentage (G) and number (H) of Foxp3⁺ CD4⁺ T cells in the BM, SP, and PDLN of AMD3100 or PBS-treated NOD mice. *p<0.05; **p<0.01.

FIG 11: Foxp3 and CD25 staining profiles. Example of Foxp3 versus CD25 staining profiles of CD4+ T cells from bone marrow (BM), pancreas-draining lymph node (PDLN), and spleen (SP) of prediabetic NOD mice (15-16 weeks of age) that were given PBS or AMD3100 daily for eight days.

### DEFINITIONS

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” or “natural amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “non-natural amino acid” encompasses chemically produced or modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Autoimmunity: As used herein, the term “autoimmunity” refers to the failure of an organism to recognize its own constituent parts (down to the sub-molecular levels) as “self.” In general, autoimmunity results in an immune response against the organism's own cells and tissues. As used herein, any disease that results from such an aberrant immune response is termed an “autoimmune disease” or “autoimmune disorder.” Exemplary autoimmune disorders include, but are not limited to, those listed in below in Tables 1 and 2.

Characteristic portion: As used herein, the phrase a “characteristic portion” of a substance, in the broadest sense, is one that shares some degree of sequence and/or structural identity and/or at least one functional characteristic with the relevant intact substance. For example, a “characteristic portion” of a polynucleotide is one that contains a continuous stretch of nucleotides, or a collection of continuous stretches of nucleotides, that together are characteristic of a polynucleotide. In some embodiments, each such continuous stretch generally will contain at least 2, 5, 10, 15, 20 or more nucleotides. In some embodiments, the characteristic portion may be biologically active.

CXCL12 and/or CXCR4 Antagonist: As used herein, the term “CXCL12 and/or CXCR4 antagonist” refers to any substance that directly and/or indirectly changes, affects, alters, inhibits, and/or decreases the activity, function, stability, and/or levels of CXCL12 and/or CXCR4. CXCL12 and/or CXCR4 antagonists may inhibit, reduce, decrease, and/or abolish CXCL12 and/or CXCR4 mRNA and/or protein levels; an activity of CXCL12 and/or CXCR4; the half-life of CXCL12 and/or CXCR4 mRNA and/or protein; and/or the interaction between CXCL12 and/or CXCR4 and their natural binding partners, as measured using standard methods. mRNA levels may be determined using standard RNase protection assays and/or in situ hybridization assays, and/or protein levels may be determined using standard Western and/or immunohistochemistry analysis. In certain embodiments, CXCL12 and/or CXCR4 antagonists may negatively affect CXCR4 signaling. In certain embodiments, CXCL12 and/or CXCR4 antagonists may negatively affect CXCR4-mediated biological effects. In certain embodiments, a CXCL12 and/or CXCR4 antagonist is any substance that results in mobilization of naïve T cells and Tregs from the bone marrow to peripheral lymphoid organs. In some embodiments, CXCL12 and/or CXCR4 antagonists may inhibit, reduce, decrease, and/or abolish binding between CXCL12 and CXCR4. Thus, in some embodiments, binding between CXCL12 and CXCR4 is stronger in the absence of the CXCL12 and/or CXCR4 antagonist than in its presence. Put another way, a CXCL12 and/or CXCR4 antagonist increases the Km of binding between CXCL12 and CXCR4. CXCL12 and/or CXCR4 antagonists may be inorganic and/or organic. CXCL12 and/or CXCR4 antagonists may comprise one or more of the following: proteins, peptides, antibodies, nucleic acids, antisense oligonucleotides, ribozymes, viruses, small molecules, proteoglycans, lipids, and/or carbohydrates. CXCL12 and/or CXCR4 antagonists may be in the form of monomers, dimers, oligomers, and/or in a complex.

Diabetes: As used herein, the term “diabetes” refers to an autoimmune disorder in which the body's own immune system attacks the beta cells in the islets of Langerhans of the pancreas. In some cases, the beta cells are damaged and/or destroyed sufficiently to reduce and/or eliminate insulin production. Damage and/or destruction of the beta cells is due to defects in both central and peripheral T cell tolerance. As used herein, the term “diabetes” generally refers to “type I diabetes,” but can refer to any condition characterized by an autoimmune attack on beta cells in the pancreas.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode RNA molecules (e.g., functional RNA molecules, such as rRNAs and/or tRNAs).

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989. 4: 11-17; incorporated herein by reference), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Natural binding partner: As used herein, the term “natural binding partner” refers to any substance that binds to CXCL12 and/or CXCR4. In some embodiments, the substance binds directly, and in some embodiments, the substance binds indirectly. A natural binding partner may be a protein, nucleic acid, lipid, carbohydrate, proteoglycan, and/or small molecule that binds to either CXCL12 and/or CXCR4. A change in the interaction between CXCL12 and/or CXCR4 and a natural binding partner may manifest itself as an increased and/or decreased probability that the interaction forms and/or as an increased and/or decreased concentration of CXCL12 and/or CXCR4/natural binding partner complex within the cell. This can result in an increased and/or decreased activity of CXCL12 and/or CXCR4. The present invention identifies CXCL12 as a novel natural binding partner of CXCR4, and CXCR4 as a natural binding partner of CXCL12. One of ordinary skill in the art will appreciate that any substance that interacts with CXCL12 and/or CXCR4 can be considered a natural binding partner of CXCL12 and/or CXCR4.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methyleytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

Patient: As used herein, the terms “patient” and “subject” can be used interchangeably and refer to any organism to which a composition of this invention may be administered, e.g., for experimental, diagnostic, identification, screening, and/or therapeutic purposes. Typical subjects include animals (e.g., mammals such as mice, rats, rabbits, non-human primates, and humans).

Sample: As used herein, the term “sample” refers to any biological tissue or fluid. In some embodiments, samples include, but are not limited to, bone marrow; blood; blood cells (e.g., white blood cells, red blood cells, etc.); ascites; tissue or fine needle biopsy samples; cell-containing body fluids; free floating nucleic acids; sputum; urine; cerebrospinal fluid, peritoneal fluid; pleural fluid; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a sample is or comprises cells obtained from a patient. The cells may be, for example, from blood, bone marrow, and/or from tissue derived from solid organs, such as brain, spleen, bone, heart, vascular, lung, kidney, liver, pituitary, endocrine glands, lymph node, dispersed primary cells, tumor cells, etc. Biological samples may include sections of tissues such as frozen or fixed sections taken for histological purposes. In some embodiments, a sample may be a body fluid, including, but not limited to, blood fluids, lymph, ascitic fluids, gynecological fluids, urine, etc. Samples may be obtained from a subject by any of a wide variety of methods including biopsy (e.g., fine needle aspiration or tissue biopsy), surgery, collection of body fluid (e.g., blood, lymph, etc.), etc. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc.

Similarity: As used herein, the term “similarity” refers to the overall relatedness between polymeric molecules, e.g., between nucleic acid molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Small molecule: In general, a “small molecule” is understood in the art to be an organic molecule that is less than about 2000 g/mol in size. In some embodiments, the small molecule is less than about 1500 g/mol or less than about 1000 g/mol. In some embodiments, the small molecule is less than about 800 g/mol or less than about 500 g/mol. In some embodiments, small molecules are non-polymeric and/or non-oligomeric. In some embodiments, small molecules are not proteins, peptides, or amino acids. In some embodiments, small molecules are not nucleic acids or nucleotides. In some embodiments, small molecules are not saccharides or polysaccharides.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, diabetes) may be characterized by one or more of the following: (1) a genetic mutation associated with development of the disease, disorder, and/or condition; (2) a genetic polymorphism associated with development of the disease, disorder, and/or condition (e.g., particular human leukocyte antigen [HLA] phenotypes); (3) increased and/or decreased expression and/or activity of a protein associated with the disease, disorder, and/or condition; (4) habits and/or lifestyles associated with development of the disease, disorder, and/or condition (e.g., not having been breast fed as an infant, vitamin D deficiency in childhood); (5) a family history of the disease, disorder, and/or condition (e.g., parent with diabetes); (6) reaction to an infection (e.g., infection by one of the viruses of the Coxsackie virus family or German measles); (7) exposure to certain chemicals (e.g., Vacor (N-3-pyridylmethyl-N′-p-nitrophenyl urea), a rodenticide which selectively destroys pancreatic βcells; or ZANOSAR® (streptozotocin), an antibiotic and antineoplastic agent used in chemotherapy for pancreatic cancer, that kills β cells, resulting in loss of insulin production). In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Test substance: As used herein, the phrase “test substance” refers to any substance that may be utilized in the systems, methods, assays, and/or compositions described herein. A “test substance” may refer to one or more of the following: (1) a CXCL12 and/or CXCR4 protein, a nucleic acid encoding CXCL12 and/or CXCR4, and/or homolog, portion, variant, mutant, and/or derivative thereof; (2) a natural binding partner of CXCL12 and/or CXCR4, a nucleic acid encoding a natural binding partner of CXCL12 and/or CXCR4, and/or a homolog, portion, variant, mutant, and/or derivative thereof; and/or (3) a substance related to CXCL12 and/or CXCR4 signal transduction, and/or a homolog, portion, variant, mutant, and/or derivative thereof. In some embodiments, a test substance is a protein or peptide comprising a CXCR4-binding portion of CXCL12. In some embodiments, a test substance is a protein or peptide comprising a CXCL12-binding portion of CXCR4. In some embodiments, a test substance is a polypeptide, polynucleotide, carbohydrate, lipid, small molecule, library of any of these, and/or combination of any of these.

Therapeutically effective amount: As used herein, the term “therapeutically effective amount” means an amount of a therapeutic and/or diagnostic agent (e.g., AM3100) that is sufficient, when administered to a patient suffering from or susceptible to a disease, disorder, and/or condition, to treat and/or diagnose the disease, disorder, and/or condition.

Therapeutic agent: As used herein, the phrase “therapeutic agent” refers to any agent that, when administered to a subject, has a therapeutic and/or diagnostic effect and/or elicits a desired biological and/or pharmacological effect.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” an autoimmune disorder may refer to (I) identifying a patient that may be responsive to a particular therapeutic agent; and (2) administering that therapeutic agent to the patient. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises (1) identifying a patient that may be responsive to AMD3100 treatment; and (2) administering AMD3100 to the patient. In some embodiments, treating may involve administering a therapeutically effective amount of one or more compositions in accordance with the invention to a subject suffering from and/or susceptible to a disease, disorder, and/or condition.

### DETAILED DESCRIPTION OF CERTAIN EMBODIMENTS OF THE INVENTION

The present invention provides compositions, systems, and methods of identifying a patient who might be likely to respond to treatment with CXCL12 and/or CXCR4 antagonists, as described herein. The present invention encompasses the recognition that some autoimmune disorders (e.g., diabetes) are associated with elevated levels of CXCL12 in certain tissues and/or cells (e.g., bone marrow, blood, etc.). The present invention encompasses the recognition that some autoimmune disorders (e.g., diabetes) are associated with elevated levels of naïve T cells and/or stem cells in certain tissues and/or cells (e.g., bone marrow). The present invention encompasses the recognition that identification of patients suffering from or susceptible to an autoimmune disorder that is associated with elevated levels of CXCL12 is desirable because it allows for identification of patients who might be likely to respond to particular therapies (e.g., CXCL12 and/or CXCR4 antagonists). It will be appreciated that methods in accordance with the invention do not necessarily predict with complete accuracy whether any particular subject will exhibit a favorable response but rather indicate that subjects having certain features are more likely or less likely to exhibit a favorable response than subjects not having such features. The present invention provides novel use of CXCL12 and/or CXCR4 antagonists. The present invention provides methods of administering CXCL12 and/or CXCR4 antagonists to a patient in need thereof.

### T Cells

Lymphocytes (e.g., T cells and B cells) develop from pluripotent hematopoietic stem cells in hone marrow. T cells migrate to the thymus where they continue their development, and continue their migration to secondary lymphoid organs (e.g., lymph nodes, spleen, etc.) where they are available to interact with antigen. T cells can be distinguished from other lymphocytes, such as B cells or natural killer (NK) cells, by the presence of T cell receptors (TCRs) on their cell surface.

T cells may be considered to be either naïve, memory, or effector cells. Naïve cells are those which have not yet been stimulated by antigen since leaving the thymus. Memory cells are those which have had antigen presented to them at least once and have returned to a resting state from which they can be rapidly activated on subsequent exposure to the same antigen. Effector cells are those which, in response to presented antigen, are able to carry out specialized functions such as lysis of a target cell.

T cells kill virus-infected cells and help or inhibit responses of other white blood cells. These three functions are carried out by different classes of T cells, including cytotoxic T cells (i.e., “CD8⁺ cells”), helper T cells (i.e., “CD4 cells”), and T regulatory cells (Tregs; i.e., “CD4⁺ cells”). Cytotoxic T cells, together with B cells, are the main effector cells of the immune system. Helper T cells help cytotoxic T cells and B cells to mount responses to antigen. They can also exhibit effector function through their secreted cytokines. Tregs typically act to suppress (“suppressor T cells”) responses of other white blood cells. Tregs can be distinguished from other types of T cells by the presence of an intracellular molecule called FoxP3.

### Autoimmune Disease

Autoimmunity is the failure of an organism to recognize its own constituent parts (down to sub-molecular levels) as “self,” which results in an immune response against the organism's own tissues, cells, and molecules. Any disease that results from such an aberrant immune response is termed an “autoimmune disease” or “autoimmune disorder.” Exemplary autoimmune diseases and/or suspected autoimmune diseases include, but are not limited to, diseases presented in Table 1:

TABLE 1
Exemplary Autoimmune Diseases and Suspected Autoimmune Diseases

| Disease | Characteristics |
|---|---|
| Acute disseminated | form of encephalitis caused by an autoimmune reaction and |
| encephalomyelitis (ADEM) | typically occurring a few days or weeks after a viral infection |
| | or a vaccination |
| Addison's disease | often caused by autoimmune destruction of the adrenal cortex |
| Alopecia universalis | body's white blood cells attack hair and result in total baldness |
| Ankylosing spondylitisis | chronic, painful, progressive inflammatory arthritis affecting |
| | primarily spine and sacroiliac joints, causing eventual fusion |
| | of the spine |
| Antiphospholipid antibody | affects the blood-clotting process; causes blood clots to form |
| syndrome (APS) | in veins and/or arteries |
| Aplastic anemia | often caused by an autoimmune attack on the bone marrow |
| Autoimmune hemolytic | disorder characterized by IgM attack against red blood cells |
| anemia | |
| Autoimmune hepatitis | liver is the target of the body's own immune system |
| Autoimmune inner ear | progressive non-age-related sensorineural hearing loss and |
| disease (AIED) | sometimes vertigo |
| Autoimmune | autoimmunity and lymphoma may be related; mutation in one |
| lymphoproliferative | of the genes that regulates the death of lymphocytes |
| syndrome (ALPS) | |
| Autoimmune oophoritis | immune system attacks the female reproductive organs |
| Balo disease | rare form of multiple sclerosis; also known as “concentric |
| | sclerosis,” “encephalitis periaxialsis concentrica,” or |
| | “leukoencephalitis periaxalis concentric” |
| Behcet's disease | exact cause is unknown in this multi-system condition, where |
| | the immune system, predominantly overactive, produces |
| | inflammation in bodily tissues, primarily causing vasculitis |
| Bullous pemphigoid | chronic, autoimmune disease that primarily affects the skin |
| Cardiomyopathy | refers to a number of diseases that weaken the heart muscle |
| Chagas' disease | in the chronic phase, believed to result from homology of a T. |
| | cruzi antigen to body tissue, resulting in a delayed |
| | autoimmune reaction leading to Chagasic cardiopathy |
| | (cardiomegaly), vovulus, or constipation, and ultimately death |
| Chronic fatigue immune | disorder whose primary symptom is usually intense fatigue; |
| dysfunction syndrome | though the syndrome likely has multiple causes, some |
| (CFIDS) | maintain that autoimmune damage to the brain stem is the |
| | principal mechanism in a significant subset of cases |
| Chronic inflammatory | rare autoimmune disorder in which there is swelling of nerve |
| demyelinating | roots and destruction of the covering (myelin sheath) over the |
| polyneuropathy | nerves |
| Crohn's disease | form of inflammatory bowel disease characterized by chronic |
| | inflammation of the intestinal tract; major symptoms include |
| | abdominal pain and diarrhea |
| Cicatrical pemphigoid | also known as “mucous membrane pemphigoid” or “benign |
| | pemphigoid”; chronic autoimmune disease of the mucosal |
| | membranes and/or skin |
| Coeliac sprue-dermatitis | characterized by chronic inflammation of the proximal portion |
| herpetiformis | of the small intestine caused by exposure to certain dietary |
| | gluten proteins |
| Cold agglutinin disease | acquired autoimmune hemolytic anemia due to an IgM |
| | autoantibody usually directed against the I antigen on red |
| | blood cells |
| CREST syndrome | acronym for calcinosis, Raynaud phenomenon, esophageal |
| | dysfunction, sclerodactyly, and telangiectasia; variant of the |
| | two groups of scleroderma, localized and systemic and is a |
| | relatively stable and slow-moving form of scleroderma |
| Degos disease | rare systemic disorder that affects small and medium-sized |
| | arteries, causing occlusive arteriopathy |
| Diabetes mellitus | consequence of an autoimmune attack on the insulin- |
| | producing beta cells in the islets of Langerhans of the pancreas |
| Discoid lupus | benign, distinctive disc-shaped skin eruption |
| Dysautonomia | malfunction of the autonomic nervous system, including such |
| | disorders as postural orthostatic tachycardia syndrome |
| | (POTS); though dysautonomia appears to have multiple |
| | causes, post-viral autoimmune damage appears to be a |
| | frequent cause |
| Endometriosis | common medical condition wherein the tissue lining the |
| | uterusis found outside of the uterus, typically affecting other |
| | organs in the pelvis; can lead to serious health problems, |
| | primarily pain and infertility |
| Essential mixed | rare autoimmune disorder that may involve the blood and |
| cryoglobulinemia | various other tissues and organs |
| Fibromyalgia-fibromyositis | widespread pain and tenderness, fatigue, and exhaustion after |
| | minimal effort |
| Goodpasture's syndrome | characterized by rapid destruction of the kidneys and |
| | hemorrhaging of the lungs through autoimmune reaction |
| | against an antigen found in both organs |
| Grave's disease | the most common form of hyperthyroidism; caused by anti- |
| | thyroid antibodies that have the effect of stimulating the |
| | thyroid into overproduction of thyroid hormone |
| Guillain-Barré syndrome | acquired immune-mediated inflammatory disorder of the |
| (GBS) | peripheral nervous system; also called acute inflammatory |
| | demyelinating polyneuropathy, acute idiopathic |
| | polyradiculoneuritis, acute idiopathic polyneuritis, and |
| | Landry's ascending paralysis |
| Hashimoto's thyroiditis | common form of hypothyroidism, characterized by initial |
| | inflammation of the thyroid, dysfunction, and goiter |
| Hidradenitis suppurativa | rare skin disease in which apocrine sweat glands become |
| | severely inflamed |
| Idiopathic and/or acute | body produces anti-platelet antibodies resulting in a low |
| thrombocytopenic purpura | platelet count |
| Idiopathic pulmonary | disease of inflammation that results in scarring, or fibrosis, of |
| fibrosis | the lungs |
| IgA neuropathy | kidney disease marked by IgA glomerulonephritis due to the |
| | glomerular immune deposit formation in the kidney |
| Interstitial cytisis | urinary bladder disease characterized by any of the following |
| | symptoms, though symptoms vary greatly from patient to |
| | patient: pelvic pain, urinary frequency (as often as every 30 |
| | minutes, or even fewer), urgency, pain with sexual |
| | intercourse, and pain with urination |
| Juvenile arthritis | rheumatic autoimmune disease characterized by chronic |
| | inflammation of the synovial tissue found in joints; onset in a |
| | child under the age of 16 years |
| Kawasaki's disease | autoimmune attack on the arteries around the heart |
| Lichen planus | inflammatory autoimmune skin disease which can affect the |
| | eyes, the skin, and the mucosa lining of the mouth and |
| | genitalia |
| Lupus erythematosus | chronic autoimmune disease wherein the immune system, for |
| | unknown reasons, becomes hyperactive and attacks normal |
| | tissue; attack results in inflammation and brings about |
| | symptoms |
| Lyme disease | caused by a bacterium; after several months, approximately |
| | 60% of patients with untreated infection will begin to have |
| | intermittent bouts of arthritis, with severe joint pain and |
| | swelling; up to 5% of untreated patients may develop chronic |
| | neurological complaints months to years after infection, |
| | including shooting pains, numbness or tingling in the hands or |
| | feet, and problems-with concentration and short term memory |
| Menière disease | recurrent and usually progressive group of symptoms, |
| | including tinnitus (ringing in the ears), vertigo (dizziness), and |
| | a sensation of fullness or pressure in the ears |
| Mixed connective tissue | used to describe overlapping groups of connective tissue |
| disease (MCTD) | disorders that cannot be diagnosed in more precise terms |
| Multiple sclerosis | disorder of the central nervous system characterized by |
| | decreased nerve function due to myelin loss and secondary |
| | axonal damage |
| Myasthenia gravis | disorder of neuromuscular transmission leading to fluctuating |
| | weakness and fatigue; weakness is caused by circulating |
| | antibodies that block acetylcholine receptors at the |
| | neuromuscular junction |
| Neuromyotonia | spontaneous muscular activity resulting from repetitive motor |
| | unit action potentials of peripheral origin; develops as a result |
| | of both acquired and hereditary diseases; the acquired form is |
| | more frequent and is usually caused by antibodies against |
| | neuromuscular junction |
| Opsoclonus myoclonus | neurological disorder that results from an autoimmune attack |
| syndrome (OMS) | on the nervous system; symptoms include opsoclonus, |
| | myoclonus, ataxia, intention tremor, dysphasia, dysarthria, |
| | mutism, hypotonia, lethargy, irritability, and malaise |
| Optic neuritis | inflammation of the optic nerve that may cause a complete or |
| | partial loss of vision |
| Ord's thyroiditis | similar to Hashimoto's disease, except that the thyroid is |
| | reduced in size |
| Pemphigus vulgaris | autoimmune disorder that causes blistering and raw sores on |
| | skin and mucous membranes |
| Pernicious anemia | autoimmune disorder characterized by anemia due to |
| | malabsorption of vitamin B12 |
| Polyarthritis (in dogs) | immune reaction severely affecting the joints of dogs |
| Polychondritis | rare degenerative autoimmune disease characterized by |
| | recurrent inflammation of the cartilage in the body |
| Polymyositis and | autoimmune neuromuscular and/or connective tissue diseases |
| dermatomyositis | |
| Primary biliary cirrhosis | autoimmune disease that affects the biliary epithelial cells |
| | (BECs) of the small bile duct in the liver |
| Psoriasis | skin disorder in which rapidly-multiplying skin cells produce |
| | itchy, scaly inflamed patches on the skin |
| Polyarteritis nodosa | inflammation of the arteries resulting in damage to the walls |
| | of the arteries, thus creating a narrowing of the vessels |
| Polyglandular syndromes | group of symptoms and signs of disordered function related to |
| | one another by some anatomic, physiologic, or biochemical |
| | peculiarity affecting many glands |
| Polymyalgia rheumatica | inflammatory syndrome |
| Primary | immune disorder related to antibody deficiency |
| agammaglobulinemia | (hypogammaglobulinemia) |
| Raynaud phenomenon | patients usually report “cold fingers” accompanied by color |
| | changes of the skin (white, blue or red); most persons with RP |
| | note cold-induced numbness of the fingers and toes and |
| | occasional discomfort with a sense of hand clumsiness. |
| Rheumatoid arthritis | autoimmune disorder that causes the body's immune system to |
| | attack the bone joints |
| Reiter's syndrome | autoimmune attack on various body systems in response to a |
| | bacterial infection and the body's confusion over the HLA- |
| | B27 marker |
| Rheumatic fever | hypersensitive reaction of the immune system to group A |
| | beta-hemolytic streptococcal infection |
| Sarcoidosis | disease wherein granulomas can form anywhere in the body |
| | but particularly in the lungs |
| Schizophrenia | mental disease characterized by impairments in the perception |
| | or expression of reality and by significant social or |
| | occupational dysfunction |
| Scleroderma | chronic disease characterized by excessive deposits of |
| | collagen; progressive systemic scleroderma can be fatal |
| Sjogren's syndrome | autoimmune disorder in which immune cells attack and |
| | destroy the exocrine glands that produce tears and saliva |
| Stiff person syndrome | also referred to as “Moersch-Woltmann syndrome”; rare, |
| | severe autoimmune neurologic disease involving the central |
| | nervous system |
| Takayasu's arteritis | disorder that results in the narrowing of the lumen of arteries |
| Temporal arteritis (also | inflammation of blood vessels, most commonly the large and |
| known as “giant cell | medium arteries of the head; untreated, the disorder can lead |
| arteritis”) | to significant vision loss |
| Ulcerative colitis | inflammatory disease of the bowel that usually affects the |
| | distal end of the large intestine and rectum; some medical |
| | authorities classify colitis as an autoimmune disease |
| Uveitis | uvea refers to the layer between sclera and retina; uveitis |
| | refers to inflammation of uvea |
| Vasculitis | result of chronic inflammation of the blood vessel walls |
| Vitiligo | spontaneous loss of pigment from areas of skin; pigment-free |
| | areas have few or no melanocytes; anti-melanocyte antibodies |
| | detected in some cases |
| Vulvodynia (“vulvar | pain in the vulva, often severe |
| vestibulitis”) | |
| Wegener's granulomatosis | form of vasculitis that affects the lungs, kidneys and other |
| | organs |

One of ordinary skill in the art will recognize that Table 1 presents an exemplary, not comprehensive, list of autoimmune disorders and suspected autoimmune disorders. Any disorder that is characterized by failure of an organism to recognize its own constituent parts as “self,” resulting in an immune response against an organism's own tissues, cells and molecules, can be classified as an autoimmune disorder.

Autoimmune disease may be caused by a variety of factors. In some embodiments, autoimmune disease may be initiated by a genetic predisposition. In some embodiments, autoimmune disease may be initiated by certain exogenous agents (e.g., viruses, bacteria, chemical agents, etc.). Some forms of autoimmunity arise as a result of trauma to an area usually not exposed to lymphocytes (e.g., neural tissue, lens of the eye, etc.). When tissues in these areas become exposed to lymphocytes, their surface proteins can act as antigens and trigger production of antibodies and cellular immune responses which then begin to destroy those tissues. In some embodiments, autoimmune disease develops after exposure of a subject to antigens which are antigenically similar (i.e., cross-reactive with) the subject's own tissue. For example, in rheumatic fever, an antigen of the streptococcal bacterium (which causes rheumatic fever) is cross-reactive with parts of the human heart. Antibodies cannot differentiate between bacterial components and heart muscle molecules; consequently cells with either of those antigens can be destroyed. In some embodiments, autoimmune diseases (e.g., type I diabetes, multiple sclerosis, rheumatoid arthritis, etc.) are characterized as being a result of mostly cell-mediated autoimmune response and appear to be primarily due to activity of T cells (Sinha et al., 1990, Science, 248:1380; incorporated herein by reference). In some embodiments, autoimmune diseases (e.g., myesthenia gravis, lupus erythematosus, etc.) are characterized as being a result of primarily a humoral immune response.

Type I Diabetes

Type I diabetes, which affects 1 in 500 children, is characterized by loss of insulin-producing beta cells of the islets of Langerhans of the pancreas, leading to a deficiency of insulin. The body's own immune system attacks the beta cells in the islets of Langerhans of the pancreas, destroying them or damaging them sufficiently to reduce and eventually eliminate insulin production. Currently, there are no known preventative measures that can be taken against type I diabetes. The primary cause of beta cell loss leading to type I diabetes is a T-cell mediated autoimmune attack.

Currently, type I diabetes can be treated only with administration of insulin, with careful monitoring of blood glucose levels using blood testing monitors. Type I diabetes treatment must be continued indefinitely, and constant monitoring of blood glucose levels and self-administration of insulin is extremely inconvenient for diabetic patients. Furthermore, it is often difficult for a patient to determine the appropriate dosage of insulin at any given time, because it can vary greatly depending on a patient's food intake, level of activity, etc. Administration of insufficient levels of insulin results in high levels of blood glucose, which may lead to ketosis, diabetic ketoacidosis, coma, or death. However, administration of too much insulin results in low levels of blood glucose, which may lead to seizures or episodes of unconsciousness.

Effective management of type I diabetes is important because the disease can have long-term health consequences which worsen over time. To give but a few examples, individuals with poorly controlled diabetes often heal slowly, even from small cuts, abrasions, blisters, etc. In such cases, such damage, if unnoticed, left untreated, or failing to heal, can result in an infection, which can lead to amputation. Furthermore, chronic elevation of blood glucose level leads to damage of blood vessels, which can lead to diabetic retinopathy (which can lead to blindness), diabetic neuropathy (which can lead to diabetic foot), or diabetic nephropathy (which can lead to renal failure, treatable by dialysis or by kidney transplant). Diabetic patients arc also prone to gum disease. Diabetic patients are frequently susceptible to accelerated atherosclerosis, which can lead to coronary artery disease (which can lead to heart attack), stroke, peripheral vascular disease, and diabetic myonecrosis (i.e., “muscle wasting”). In fact, people with diabetes arc 2 to 4 times more likely to suffer a stroke than people without diabetes. Diabetes complicates pregnancy and can cause nausea, poor digestion, and bloating. People with diabetes are more likely to die with pneumonia or influenza than people who do not have diabetes.

Thus, compositions and methods of inhibiting and/or delaying the onset of diabetes are desirable because these could inhibit and/or delay the onset of negative long-term health consequences commonly associated with diabetes. Furthermore, prior to treatment with such compositions, it is desirable to identify whether a patient might be likely to respond to these compositions. Such identification allows for “personalized” treatment and may avoid administering these compositions to patients who will not be responsive to a composition, thus avoiding potentially adverse side effects.

The non-obese diabetic (NOD) mouse, which is predisposed to develop type I diabetes, has served as a model for studying the mechanism, pathogenesis, and interventions of the human disease (Anderson and Bluestone, 2005, Ann. Rev. Immunol., 23:447). As in humans, development of type I diabetes in NOD mice is age-dependent and progresses through two distinct stages: insulitis and diabetes (Anderson and Bluestone, 2005, supra; and Andre et al., 1996, Proc. Natl. Acad. Sci., USA, 93:2260; both of which are incorporated herein by reference). As early as 3-4 weeks of age, mononuclear infiltrates begin to surround the islets (peri-insulitis). Over the next few weeks, the infiltrates invade the islets, resulting in insulitis. By 12-14 weeks of age, insulitis starts to shift from “benign” to “aggressive.” As β-cells are destroyed, overt diabetes develops. Like many spontaneous autoimmune diseases, the development of type I diabetes is variable. In NOD mice, insulitis occurs in all mice (complete penetrance), but diabetes appears more frequently and at younger age in females than in males. It is generally believed that defects in multiple factors and processes contribute to type I diabetes.

The primary cause of type I diabetes is destruction of β-cells by autoreactive T cells. Studies have shown that both CD4⁺ and CD8⁺ T cells can directly mediate islet destruction and transfer the disease. Thus, development and activation of autoreactive T cells and their infiltration of islets and destruction of β-cells are important for disease development. In NOD mice, a combination of a unique major histocompatibility complex (MHC) class II allele (IA^{g7}) and a defect in the programmed cell death pathway is thought to permit autoreactive T cells to escape negative selection in the thymus (Dclovitch and Singh, 1997, Immunity, 7:727; and Kishimoto and Sprent, 2001, Nat. Immunol., 2:1025; and Zucchelli et al., 2005, Immunity, 22:385; all of which are incorporated herein by reference). In the periphery, additional defects in the peripheral tolerance mechanisms fail to suppress activation of autoreactive T cells (Hong et al., 2001, Nat. Med., 7:1052; Salomon et al., 2000, Immunity, 12:431; and Sharif et al., 2001, Nat. Med., 7:1057; all of which are incorporated herein by reference). Infiltration of islets and destruction of β-cells by autoreactive T cells lead to loss of insulin production and control of blood glucose level, and eventually diabetes.

Although development and activation of autoreactive T cells have been extensively investigated in NOD mice, relatively little is known about the control of T cell trafficking, such as infiltration of islets. Because trafficking of autoreactive T cells into the islets is a pre-requisite for β-cell destruction, alteration in T cell trafficking is expected to affect disease progression in NOD mice. Consistent with this notion, serum levels of chemokines CCL3, CCL4, and CXCL10 are significantly elevated in NOD mice during disease progression (Hanifi-Moghaddam et al., 2006, Diabet. Med., 23:156; and Shigihara et al., 2006, J. Autoimmun., 26:66; both of which are incorporated herein by reference). Polymorphisms in several chemokines and chemokine receptor, including CXCL12, also known as stromal cell derived factor-1 (SDF-1), have been found in human type I diabetes mellitus (Dubois-Laforguc et al., 2001, Diabetes, 50:1211; Ide et al., 2003, Hum. Immunol., 64:973; Kawasaki et al., 2004, Ann. NY Acad. Sci., 1037:79; and Yang et al., 2004, Cytokine, 26:114; all of which are incorporated herein by reference). In NOD mice, blocking CXCL12 with antibodies leads to a moderate delay of onset of type I diabetes (Matin et al., 2002, Immunology, 107:222; incorporated herein by reference). Likewise, treatment of prediabetic NOD mice with G-CSF, a known suppressor of CXCL12 expression (Petit et al., 2002, Nat. Immunol., 3:687; and Semerad et al., 2005, Blood, 106:3020; both of which are incorporated herein by reference), significantly delays onset of type I diabetes (Kared et al., 2005, Diabetes, 54:78; and Hadaya et al., 2005, J. Autoimmun., 24:125; both of which are incorporated herein by reference). Therefore, inhibition of CXCL12 appears to slow down disease progression in NOD mice. However, the precise role of chemokines, including CXCL12, in disease progression has not been fully investigated.

In addition to T cells, other factors and processes also modulate disease progression as expected from the complex, multi-stage development of type I diabetes. Indeed, almost 20 idd loci have been linked to disease progression. While some of these loci likely modulate development, activation, trafficking and function of autoreactive T cells, some might modulate disease progression independent of T cells. For example, during the course of disease progression, as the islet is being destroyed, it is likely that normal repair and regeneration mechanisms are activated to replace destructed β-cells. Recent studies have provided some of most compelling evidence suggesting the presence of such repair/regeneration mechanisms. Ryu et al. showed that a significant fraction of diabetic NOD mice can be cured of the disease by a combination of complete Freund adjuvant (CFA) injection and repeated adoptive transfer of MHC class I-matched splenocytes (Ryu et al., 2001, J. Clin. Invest., 108:63; incorporated herein by reference). Follow-up studies demonstrated that diabetic mice were cured due to regeneration of islets (Kodama et al., 2003, Science, 302:1223; incorporated herein by reference), although whether donor splenocytes contribute to islet regeneration is controversial (Chong et al., 2006, Science, 311:1774; Nishio et al., 2006, Science, 311:1775; Suri et al., 2006, Science, 311:1778; all of which are incorporated herein by reference). Because bone marrow-derived stem cells appear to initiate pancreatic regeneration (Hess et al., 2003, Nat. Biotech., 21:763; incorporated herein by reference), chemokines that regulate stem cell mobilization in bone marrow may modulate islet regeneration and therefore development of diabetes. However, there is no evidence supporting a direct link between chemokines, T cell and stem cell trafficking/mobilization, and type I diabetes. In addition, the role of CFA, which by itself can prevent diabetes in NOD mice, remains enigmatic.

T Cells and Autoimmune Disease

Thus, the present invention encompasses the discovery of the mechanism by which CXCL12 is involved in onset of type I diabetes. As shown in the Exemplification below, the present invention encompasses the recognition that expression of chemokine CXCL12 is elevated in bone marrow in NOD mice, resulting in an accumulation of both T cells and hematopoietic stem cells (HSCs) in bone marrow. The present invention encompasses the recognition that CXCL12 expression may be elevated in any one of a variety of tissues (e.g., bone marrow) in subjects suffering from and/or susceptible to diabetes. Treatment of NOD mice with CFA inhibits CXCL12 expression as well as T cell accumulation in bone marrow. The present invention encompasses the recognition that inhibition of CXCL12 activity with a CXCR4 antagonist (e.g., AMD3100) mobilizes both T cells and stem cells from bone marrow to peripheral lymphoid tissues, and significantly delays the onset of insulitis and diabetes in NOD mice. For example, AMD3100 (1,1′-[1,4-phenylenebis(methylene)]-bis-1,4,8,11-tetraazacyclotetradecane octahydrochloride dihydrate; also known as Plcrixafor and/or JM 3100) is a macrocyclic small molecule that can function as a CXCR4 antagonist (De Clercq, 2003, Nat. Rev. Drug Discov., 2:581; incorporated herein by reference). The present invention encompasses the recognition that elevated levels of CXCL12 expression promote type I diabetes in NOD mice and suggests a common mechanism by which various cell types and processes (e.g., chemokines, T cells, stem cells, trafficking, and/or mobilization) all contribute to disease progression. The present invention encompasses the recognition that AMD3100 may be utilized for treatment and/or prophylaxis of type I diabetes in humans.

While the specific experiments described herein are related to type I diabetes, systems, methods, and compositions in accordance with the present invention are relevant for and applicable to any autoimmune disease, as described herein. Thus, the present invention encompasses the recognition that CXCL12 and/or CXCR4 may be generally involved in autoimmune disease. The present invention encompasses the recognition that expression of chemokine CXCL12 may be elevated in any one of a variety of tissues and/or cell types (e.g., bone marrow) in subjects suffering from and/or susceptible to autoimmune disease, and that such elevated expression may result in an accumulation of both T cells and hematopoietic stem cells (HSCs) in that tissue (e.g., bone marrow). The present invention encompasses the recognition that inhibition of CXCL12 activity with a CXCR4 antagonist (e.g., AMD3100) mobilizes both T cells and stem cells from the bone marrow to peripheral lymphoid tissues, and may delay the onset of autoimmune disease. The present invention encompasses the recognition that elevated levels of CXCL12 expression and/or activity may promote autoimmune disease and suggests a common mechanism by which various cell types and processes (e.g., chemokines, T cells, stem cells, trafficking, and/or mobilization) all contribute to disease progression. The present invention encompasses the recognition that AMD3100 may be utilized for treatment and/or prophylaxis of autoimmune disease in humans.

CXCL12 and/or CXCR4 Antagonists

Chemokines are a family of small cytokines. Proteins are classified as chemokines according to shared structural characteristics such as small size (they are all approximately 8-10 kD in size), and the presence of four cysteine residues in conserved locations that are key to forming their 3-dimensional shape. Their name is derived from their ability to induce directed chemotaxis in nearby responsive cells (i.e., they are chemotactic cytokines). However, these proteins have historically been known under several other names including the SIS family of cytokines, SIG family of cytokines, SCY family of cytokines, Platelet factor-4 superfamily or intercrines. Some chemokines are considered pro-inflammatory and can be induced during an immune response to promote cells of the immune system to a site of infection, while others arc considered homeostatic and are involved in controlling migration of cells during normal processes of tissue maintenance or development. For example, some homeostatic chemokines direct lymphocytes to lymph nodes. Chemokines are found in all vertebrates, some viruses, and some bacteria, but to date, none have been described for other invertebrates. Chemokines exert their biological effects by interacting with G protein-linked transmembrane receptors called chemokine receptors that are selectively found on the surfaces of their target cells.

CXCL12, officially designated “chemokine (C-X-C motif) ligand 12,” and also known as SDF-1 (stromal cell-derived factor-1), is small cytokine belonging to the chemokine family. It is produced in two forms (i.e., SDF-1α/CXCL12a and SDF-1β/CXCL12b) by alternate splicing of the same gene (De La Luz Sierra et al., 2004, Blood, 103:2452; incorporated herein by reference). Chemokines are characterized by, the presence of four conserved cysteines which form two disulfide bonds. CXCL12 belongs to the group of CXC chemokines, whose initial pair of cysteines is separated by one intervening amino acid. CXCL12 is chemotactic for lymphocytes and has been implicated as an important cell coordinator during development (Blcul et al., 1996, J. Exp. Med., 184:1101; Ara et al., 2003, Proc. Natl. Acad. Sci., USA, 100:5319; Askari et al., 2003, Lancet, 362:697; and Ma et al., 1998, Proc. Natl. Acad. Sci., USA, 95:9448; all of which are incorporated herein by reference). During embryogenesis CXCL12 directs migration of hematopoietic cells from fetal liver to bone marrow. CXCL12 knockout mice die before birth or within just 1 hour of life. Additionally, CXCL12 alters also the electrophysiology of neurons. CXCL12 was shown to be expressed in many tissues in mice (e.g., brain, thymus, heart, lung, liver, kidney, spleen, and bone marrow).

CXC chemokine receptors are integral membrane proteins that specifically bind and respond to cytokines of the CXC chemokine family. There are currently seven known CXC chemokine receptors in mammals, named CXCR1 through CXCR7.

The receptor for CXCL12 is CXCR4, which was previously called “fusin” (Bleul et al., 1996, Nature, 382:829; incorporated herein by reference). Both CXCL12 and CXCR4 show high sequence identity between mouse and human (i.e., 99% and 90%, respectively). CXCR4 is utilized by HIV-1 to gain entry into target cells. CXCR4 has a wide cellular distribution, with expression on most immature and mature hematopoietic cell types (e.g., T cells, B cells, neutrophils, monocytes, dendritic cells, Langerhans cells, and macrophages). In addition, CXCR4 can also be found on vascular endothelial cells and neuronal cells.

In general, a CXCL12 and/or CXCR4 antagonist is any substance that negatively affects the ability of CXCL12 to bind to CXCR4 (i.e., “the CXCL12-CXCR4 interaction”). In certain embodiments, CXCR4 antagonists may negatively affect CXCR4 signaling. In certain embodiments, CXCR4 antagonists may negatively affect CXCR4-mediated biological effects. In certain embodiments, CXCR4 and/or CXCL12 antagonists may negatively affect the stability of CXCR4 and/or CXCL12 mRNA and/or protein. In certain embodiments, a CXCL12 and/or CXCR4 antagonist is any substance that results in mobilization of naïve T cells and Tregs from bone marrow to peripheral lymphoid organs.

Activities of CXCL12 and/or CXCR4 Antagonists

A CXCL12 and/or CXCR4 antagonist according to the present invention may be one which exerts its modulatory effect upstream, downstream, and/or directly on CXCL12 and/or CXCR4.

In some embodiments, CXCL12 and/or CXCR4 antagonists may bind directly to CXCL12 and/or CXCR4 and affect the ability of CXCL12 and/or CXCR4 to interact with their natural binding partners. In certain embodiments, binding of a CXCL12 and/or CXCR4 antagonist to CXCL12 blocks the interaction between CXCL12 and its natural binding partners (for example, the interaction between CXCL12 and CXCR4). In certain embodiments, binding of a CXCL12 and/or CXCR4 antagonist to CXCR4 blocks the interaction between CXCR4 and its natural binding partners (for example, the interaction between CXCL12 and CXCR4). However, a modulator need not necessarily bind directly to a catalytic and/or binding site, and may bind, for example, to an adjacent site, such as an adjacent site in the CXCL12 and/or CXCR4 polypeptide. A CXCL12 and/or CXCR4 antagonist may even bind to another substance (for example, a protein, lipid, carbohydrate, etc. which is complexed with CXCL12 and/or CXCR4), so long as its binding modulates CXCL12 and/or CXCR4 activity.

In some embodiments, a CXCL12 and/or CXCR4 antagonist may bind to and/or compete for one or more sites on a relevant molecule, for example, a site important for signal transduction and/or a binding site for a natural binding partner. In some embodiments, a CXCL12 and/or CXCR4 antagonist interferes with and/or inhibits binding of CXCL12 to CXCR4. In certain embodiments, a CXCL12 and/or CXCR4 antagonist competes for a CXCL12-binding region of CXCR4. In some embodiments, a CXCL12 and/or CXCR4 antagonist competes for a CXCR4-binding region of CXCL12.

In some embodiments, CXCL12 and/or CXCR4 antagonists arc substances which bind to and/or block domains necessary for signal transduction within T cells. In some embodiments, CXCL12 and/or CXCR4 antagonists are short peptides comprising sequences of CXCL12 and/or CXCR4 that have dominant-negative activity. In some embodiments, such peptides may not block CXCL12 and/or CXCR4 activity per se, but may displace CXCL12 and/or CXCR4 from sites of action, which may indirectly modulate CXCL12 and/or CXCR4 activity.

In certain embodiments, CXCL12 and/or CXCR4 antagonists may function by altering the activity and/or expression of CXCL12 and/or CXCR4 activators, such as RNAi-inducing entities. RNAi inducing entities are described in further detail below, in the section entitled “Nucleic Acid CXCR12or CXCR4 Antagonists.”

In some embodiments, CXCL12 and/or CXCR4 antagonists may comprise the dimerization domain of CXCR4, which may block and/or reduce CXCR4 activity. In some embodiments, CXCL12 and/or CXCR4 antagonists may comprise an entity (e.g., small molecule, protein, etc.) that inhibits dimerization of CXCR4. Dimerization of CXCR4 is typically dependent upon CXCL12 binding to CXCR4 and is described in further detail in Babcock et al., 2003, J. Biol. Chem., 278:3378; and in Mellado et al., 2006, Methods Mol. Biol., 332:141 (both of which are incorporated herein by reference).

In some embodiments, CXCL12 and/or CXCR4 antagonists function to modulate expression, stability, and/or cellular levels of CXCL12 and/or CXCR4. For example, Smith el al. describe nucleotide inhibitors of CXCL12 and/or CXCR4 expression that target CXCL12 and/or CXCR4 mRNAs for degradation (Smith et al., 2004, Cancer Res., 64:8604; incorporated herein by reference). A small molecule compound, ampelopsin decrease CXCR4 on the surface of human peripheral mononuclear cells (PBMCs) by induction internalization after binding to it (Liu et al., 2004, Biomed. Environ. Sci., 17:153; incorporated herein by reference).

Alternatively or additionally, CXCL12 and/or CXCR4 antagonists in accordance with the invention affect CXCL12 and/or CXCR4 levels by decreasing transcription and/or translation of CXCL12 and/or CXCR4 and/or natural binding partners of CXCL12 and/or CXCR4. In some embodiments, CXCL12 and/or CXCR4 antagonists may affect RNA and/or protein half-life, for example, by directly affecting mRNA and/or protein stability. In certain embodiments, CXCL12 and/or CXCR4 antagonists in accordance with the invention cause mRNA and/or protein to be more and/or less accessible and/or susceptible to nucleases, proteases, and/or the proteasome.

In some embodiments, CXCL12 and/or CXCR4 antagonists in accordance with the invention affect processing of mRNAs encoding CXCL12 and/or CXCR4 and/or natural binding partners of CXCL12 and/or CXCR4. For example, CXCL12 and/or CXCR4 antagonists may function at the level of pre-mRNA splicing, 5′ end formation (e.g., capping), 3′ end processing (e.g., cleavage and/or polyadenylation), nuclear export, and/or association with the translational machinery and/or ribosomes in the cytoplasm.

In some embodiments, CXCL12 and/or CXCR4 antagonists in accordance with the invention affect translational control and/or post-translational modification of CXCL12 and/or CXCR4 and/or natural binding partners of CXCL12 and/or CXCR4. For example, CXCL12 and/or CXCR4 antagonists may function at the level of translation initiation, elongation, termination, and/or recycling. In some embodiments, CXCL12 and/or CXCR4 antagonists may function at the step of protein folding into secondary, tertiary, and/or quaternary structures. Alternatively or additionally, CXCL12 and/or CXCR4 antagonists may function at the level of intracellular transport (e.g., ER to Golgi transport, intra-Golgi transport, Golgi to plasma membrane transport, and/or secretion from a cell). In some embodiments, CXCL12 and/or CXCR4 antagonists may function at the level of post-translational modification (e.g., cleavage of signal sequences and/or addition of entities such as methyl groups, phosphates, glycan moieties, acetyl groups, etc.).

In some embodiments, CXCL12 and/or CXCR4 antagonists in accordance with the invention may cause levels of CXCL12 and/or CXCR4 mRNA, levels of CXCL12 and/or CXCR4 protein, activit(ies) of CXCL12 and/or CXCR4 protein, half-li(ves) of CXCL12 and/or CXCR4 mRNA, half-lives) of CXCL12 and/or CXCR4 protein, binding of CXCL12 and/or CXCR4 mRNA to natural binding partners, and/or binding of CXCL12 and/or CXCR4 protein to natural binding partners to decrease by at least 5%, at least 10%, at least 20%, at least 30%, at least 40%, at least 50%, at least 60%, at least 80%, at least 90%, at least 95%, or about 100%.

CXCL12 and/or CXCR4 antagonists in accordance with the present invention may function by altering and/or disrupting the distribution of CXCL12 and/or CXCR4 mRNA and/or protein throughout a subject. For example, a CXCL12 and/or CXCR4 antagonist may cause CXCL12 and/or CXCR4 to preferentially accumulate in certain organs, tissues, cells, and/or subcellular locales. In some embodiments, such disrupted distribution may effectively lead to overexpression of CXCL12 and/or CXCR4 in certain organs, tissues, cells, and/or subcellular locales and underexpression of CXCL12 and/or CXCR4 in other organs, tissues, cells, and/or subcellular locales. In some embodiments, such CXCL12 and/or CXCR4 may act on a factor responsible for regulating proper CXCL12 and/or CXCR4 gene expression (e.g., transcription factor, splicing factor, nuclear export factor, post-translational processing factor, etc.). To give but one example, CXCL12 and/or CXCR4 antagonists may function by inhibiting accumulation of CXCL12 and/or CXCR4 in bone marrow.

CXCL12 and/or CXCR4 antagonists in accordance with the present invention may have any activity described above. According to the present invention, CXCL12 and/or CXCR4 antagonists may be small molecules, proteins (e.g., peptides, antibodies, etc.), nucleic acids (e.g., antisense oligonucleotides, ribozymes, siRNAs, etc.), lipids, carbohydrates, viruses, etc., as described in further detail below. The present invention provides novel CXCL12 and/or CXCR4 antagonists, identified in accordance with any of the methods described below.

Small Molecule CXCL12 and/or CXCR4 Antagonists

In some embodiments, a CXCL12 and/or CXCR4 antagonist in accordance with the present invention may be a small molecule. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol.

One of ordinary skill in the art will appreciate that any small molecule that negatively affects the ability of CXCL12 to bind to CXCR4 is a CXCL12 and/or CXCR4 antagonist in accordance with the present invention. Any small molecule that negatively affects activity of CXCR12 and/or CXCR4 is a CXCL12 and/or CXCR4 antagonist in accordance with the present invention. In certain embodiments, a small molecule that, upon administration to a subject, causes mobilization of naïve T cells and Tregs from bone marrow to peripheral lymphoid organs is a CXCL12 and/or CXCR4 antagonist in accordance with the present invention.

In some embodiments, exemplary small molecule CXCL12 and/or CXCR4 antagonists include, but are not limited to, AMD3100 (Rubin et al., 2003, Proc. Natl. Acad. Sci., USA, 100:13513; incorporated herein by reference), KRH-1636 (Ichiyama et al., 2003, Proc. Natl. Acad. Sci., USA, 100:4185; incorporated herein by reference), KRH-3955, KRH-3140 (Tanaka et al., 2006, “Development of Novel Orally Bioavailable CXCR4 Antagonists, KRH-3955 and KRH-3140: Binding Specificity, Pharmacokinetics and Anti-HIV-1 Activity in vivo and in vitro,” 13^{th} Conference on Viruses and Opportunistic Infections, February 2006; incorporated herein by reference), KRH-2731 (Murakami et al., “KRH-2731: An Orally Bioavailable CXCR4 Antagonist Is a Potent Inhibitor of HIV-1 Infection,” 11^{th} Conference on Viruses and Opportunistic Infections, February 2004; incorporated herein by reference), AMD3465 (Hu et al., 2006, Am. J. Pathol., 169:424; incorporated herein by reference), T134 (Gotoh et al., 2000, Abstr. Intersci. Coq: Antimicrob. Agents Chemother. Intersci. Conf. Antimicrob. Agents Chemother., 40:180; incorporated herein by reference), AMD2763 (Horzinek et al, 1999, J. Virol., 76:6346; incorporated herein by reference), AMD070 (Schols et al., “In vitro Anti-HIV Activity Profile of AMD887, a Novel CCR5 Antagonist, in Combination with the CXCR4 Inhibitor AMD070,” 11^{th} Conference on Retroviruses and Opportunistic Infections, February 2004; incorporated herein by reference), tannic acid (Chen et al., 2003, Clin. Cancer Res., 9:3115; incorporated herein by reference) and/or NSC 651016 (Schneider et al., 2002, Clin. Cancer Res., 8:3955; incorporated herein by reference).

Protein CXCL12 and/or CXCR4 Antagonists

In some embodiments, a CXCL12 and/or CXCR4 antagonist in accordance with the present invention may be a protein, including polypeptides, peptides, antibodies, glycoproteins, lipoproteins, etc.

In certain embodiments, peptides range from about 5 to about 100, about 10 to about 75, about 15 to about 50, or about 20 to about 25 amino acids in size. In some embodiments, a peptide sequence can be based on the sequence of a protein. In some embodiments, a peptide sequence can be a random arrangement of amino acids.

The terms “polypeptide” and “peptide” are used interchangeably herein, with “peptide” typically referring to a polypeptide having a length of less than about 100 amino acids. Polypeptides may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, farnesylation, sulfation, etc.

One of ordinary skill in the art will appreciate that any protein or peptide that negatively affects the ability of CXCL12 to bind to CXCR4 is a CXCL12 and/or CXCR4 antagonist in accordance with the present invention. Any protein or peptide that negatively affects the activity of CXCR12 and/or CXCR4 is a CXCL12 and/or CXCR4 antagonist in accordance with the present invention. In certain embodiments, a protein or peptide that, upon administration to a subject, causes mobilization of naïve T cells and Tregs from bone marrow to peripheral lymphoid organs is a CXCL12 and/or CXCR4 antagonist in accordance with the present invention.

In some embodiments, exemplary protein and/or peptide CXCL12 and/or CXCR4 antagonists include, but are not limited to, T-20 (Naicker et al., 2004, Org. Biomol. Chem., 5:660; incorporated herein by reference), T-22 (Owen et al., 2002, J. Med. Virol., 68:147; incorporated herein by reference), T-140 (Tamamura et al., 2003, FEBS Lett., 550:79; and Tamamura and Fujii, 2004, Curr. Drug Targets Infect. Disord., 4:103; both of which are incorporated herein by reference), TE-14011 (Takenaga et al., 2004, Biochem. Biophys. Res. Commun., 320:226; incorporated herein by reference), TC14012 (Fujii and Tamamura, 2001, Curr. Opin. Investig. Drugs, 2:1198; incorporated herein by reference), TN14003 (Tamamura et al., 2004, FEBS Lett., 569:99; and Liang et al., 2004, Cancer Res., 64:4302; incorporated herein by reference), CTCE-9908 (Kim, “Inhibition of Murine Osteosarcoma Lung Metastasis using the CXCR4 antagonist, CTCE-9908,” Am. Assoc. Canc. Res. 96^{th} Annual Meeting, April 2005; incorporated herein by reference), FC131 (Fujii et al., 2003, Angew Chem. Int. Ed. Engl., 42:3251; incorporated herein by reference), TE14011, and cyclopentapeptides (V\dot{a}benø et al., 2006, Chem. Biol. Drug Design, 67:346; incorporated herein by reference).

In sonic embodiments, a CXCL12 and/or CXCR4 antagonist may be an antibody and/or characteristic portion thereof. One of ordinary skill in the art will appreciate that any antibody that affects the ability of CXCL12 to bind CXCR4 can be used in accordance with the present invention. Any antibody that negatively affects the activity of CXCR12 and/or CXCR4 is a CXCL12 and/or CXCR4 antagonist in accordance with the present invention. In some embodiments, antibodies that, upon administration to a subject, cause mobilization of naïve T cells and Tregs from bone marrow to peripheral lymphoid organs is a CXCL12 and/or CXCR4 antagonist in accordance with the present invention.

In some embodiments, a CXCL12 and/or CXCR4 antagonist is an antibody that inhibits the ability of CXCL12 and CXCR4 to interact with one another. For example, in certain embodiments, a CXCL12 and/or CXCR4 antagonist is an antibody that binds to CXCL12 and/or CXCR4 at the interface involved in CXCL12/CXCR4 binding, thereby inhibiting the ability of CXCL12 and CXCR4 to interact with one another. In certain embodiments, a CXCL12 and/or CXCR4 antagonist is an antibody that does not bind to CXCL12 and/or CXCR4 at the interface involved in CXCL12/CXCR4 binding, but instead binds to a different region of CXCL12 and/or CXCR4 in order to inhibit the ability of CXCL12 and CXCR4 to interact with one another.

In some embodiments, CXCL12 and/or CXCR4 antagonist is an antibody that inhibits the ability of CXCR4 to dimerize. For example, in certain embodiments, a CXCL12 and/or CXCR4 antagonist is an antibody that binds to CXCR4 at the interface involved in CXCR4 dimerization, thereby inhibiting dimerization. In certain embodiments, a CXCL12 and/or CXCR4 antagonist is an antibody that does not bind to CXCR4 at the interface involved in dimerization, but instead binds to a different region of CXCR4 in order to inhibit dimerization.

The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE.

As used herein, an antibody fragment (i.e., characteristic portion of an antibody) refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments.

An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g., “humanized”) and single chain (recombinant) antibodies. In some embodiments, antibodies may have reduced effector functions and/or bispecific molecules. In some embodiments, antibodies may include fragments produced by a Fab expression library.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. A polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

A F(ab′)2 fragment is an antibody fragment essentially equivalent to that obtained from immunoglobulins by digestion with an enzyme pepsin at pH 4.0-4.5. A fragment may be recombinantly produced.

A Fab′ fragment is an antibody fragment essentially equivalent to that obtained by reduction of a disulfide bridge or bridges joining the two heavy chain pieces in a F(ab′)2 fragment. A Fab′ fragment may be recombinantly produced.

A Fab fragment is an antibody fragment essentially equivalent to that obtained by digestion of immunoglobulins with an enzyme (e.g., papain). A Fab fragment may be recombinantly produced. A heavy chain segment of a Fab fragment is the Fd piece.

Nucleic Acid CXCR12 or CXCR4 Antagonists

In some embodiments, a CXCL12 and/or CXCR4 antagonist in accordance with the present invention may be a nucleic acid (e.g., RNAi-inducing agents, ribozymes, tRNAs, aptamers, etc.). Any nucleic acid that negatively affects the ability of CXCL12 to bind to CXCR4 is a CXCL12 and/or CXCR4 antagonist in accordance with the present invention. Any nucleic acid that negatively affects the activity of CXCR12 and/or CXCR4 is a CXCL12 and/or CXCR4 antagonist in accordance with the present invention. In certain embodiments, a nucleic acid that, upon administration to a subject, causes mobilization of naïve T cells and Tregs from bone marrow to peripheral lymphoid organs is a CXCL12 and/or CXCR4 antagonist in accordance with the present invention.

RNA interference (RNAi) is an evolutionarily conserved process in which presence of an at least partly double-stranded RNA molecule in a eukaryotic cell leads to sequence-specific inhibition of gene expression. RNAi was originally described as a phenomenon in which the introduction of long dsRNA (typically hundreds of nucleotides) into a cell results in degradation of mRNA containing a region complementary to one strand of the dsRNA (U.S. Pat. No. 6,506,559; and Fire et al., 1998, Nature, 391:806; both of which are incorporated herein by reference). Subsequent studies in Drosophila showed that long dsRNAs are processed by an intracellular RNase III-like enzyme called Dicer into smaller dsRNAs primarily comprised of two approximately 21 nucleotide (nt) strands that form a 19 base pair duplex with 2 nt 3′ overhangs at each end and 5′-phosphate and 3′-hydroxyl groups (see, e.g., PCT Publication WO 01/75164; U.S. Patent Publications 2002/0086356 and 2003/0108923; Zamore el al., 2000, Cell, 101:25; and Elbashir et al., 2001, Genes Dev., 15:188; all of which are incorporated herein by reference).

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. An siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Considerable progress towards the practical application of RNAi was achieved with the discovery that exogenous introduction of siRNAs into mammalian cells can effectively reduce expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of an siRNA is perfectly complementary to its target gene and mRNA transcript over at least 17-19 contiguous nucleotides, and typically the two strands of an siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide, is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g., Reynolds et al., 2004, Nat. Biotechnol., 22:326; incorporated herein by reference).

It will be appreciated that molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., Yuan et al., 2004, Nucl. Acids. Res., 32:W130; and Santoyo et al., 2005, Bioinformatics, 21:1376; both of which are incorporated herein by reference).

As will be appreciated by one of ordinary skill in the art, RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Additional structures, referred to as short hairpin RNAs (shRNAs), arc capable of mediating RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. While shRNAs can be delivered exogenously to cells, more typically intracellular synthesis of shRNA is achieved by introducing a plasmid or vector containing a promoter operably linked to a template for transcription of the shRNA into the cell, e.g., to create a stable cell line or transgenic organism.

While sequence-specific cleavage of target mRNA is currently the most widely used means of achieving gene silencing by exogenous delivery of RNAi-inducing entities to cells, additional mechanisms of sequence-specific silencing mediated by short RNA entities are known. For example, post-transcriptional gene silencing mediated by RNAi-inducing entities can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

Thus the exact mechanism by which an RNAi-inducing entity inhibits gene expression appears to depend, at least in part, on the structure of the duplex portion of the RNAi-inducing entity and/or the structure of the hybrid formed by one strand of the RNAi-inducing entity and a target transcript. RNAi mechanisms and the structure of various RNA molecules known to mediate RNAi, e.g., siRNA, shRNA, miRNA and their precursors, have been extensively reviewed (see, e.g., Dykxhhorn et al., 2003, Nat. Rev. Mol. Cell Biol., 4:457; Hannon et al., 2004, Nature, 431:3761; and Meister et al., 2004, Nature, 431:343; all of which are incorporated herein by reference). It is to be expected that future developments will reveal additional mechanisms by which RNAi may be achieved and will reveal additional effective short RNAi-inducing entities. Any currently known or subsequently discovered RNAi-inducing entities are within the scope of the present invention.

An RNAi-inducing entity that is delivered according to methods in accordance with the invention and/or is present in a composition in accordance with the invention may be designed to silence any eukaryotic gene. The gene can be a mammalian gene, e.g., a human gene. The gene can be a wild type gene, a mutant gene, an allele of a polymorphic gene, etc. In certain embodiments, CXCL12 and/or CXCR4 antagonist is an RNAi-inducing entity that targets CXCL12. The following sequences may be used to design RNAi-inducing entities that target CXCL12, in accordance with the guidelines described herein

H. sapiens CXCL12, transcript variant 2, mRNA (GI 76563934):

(SEQ ID NO.: 1)
GCACTTTCACTCTCCGTCAGCCGCATTGCCCGCTCGGCGTCCGGCCCCCGACCCG
CGCTCGTCCGCCCGCCCGCCCGCCCGCCCGCGCCATGAACGCCAAGGTCGTGGTC
GTGCTGGTCCTCGTGCTGACCGCGCTCTGCCTCAGCGACGGGAAGCCCGTCAGCC
TGAGCTACAGATGCCCATGCCGATTCTTCGAAAGCCATGTTGCCAGAGCCAACGT
CAAGCATCTCAAAATTCTCAACACTCCAAACTGTGCCCTTCAGATTGTAGCCCGG
CTGAAGAACAACAACAGACAAGTGTGCATTGACCCGAAGCTAAAGTGGATTCAG
GAGTACCTGGAGAAAGCTTTAAACAAGAGGTTCAAGATGTGAGAGGGTCAGACG
CCTGAGGAACCCTTACAGTAGGAGCCCAGCTCTGAAACCAGTGTTAGGGAAGGG
CCTGCCACAGCCTCCCCTGCCAGGGCAGGGCCCCAGGCATTGCCAAGGGCTTTGT
TTTGCACACTTTGCCATATTTTCACCATTTGATTATGTAGCAAAATACATGACATT
TATTTTTCATTTAGTTTGATTATTCAGTGTCACTGGCGACACGTAGCAGCTTAGAC
TAAGGCCATTATTGTACTTGCCTTATTAGAGTGTCTTTCCACGGAGCCACTCCTCT
GACTCAGGGCTCCTGGGTTTTGTATTCTCTGAGCTGTGCAGGTGGGGAGACTGGG
CTGAGGGAGCCTGGCCCCATGGTCAGCCCTAGGGTGGAGAGCCACCAAGAGGGA
CGCCTGGGGGTGCCAGGACCAGTCAACCTGGGCAAAGCCTAGTGAAGGCTTCTC
TCTGTGGGATGGGATGGTGGAGGGCCACATGGGAGGCTCACCCCCTTCTCCATCC
ACATGGGAGCCGGGTCTGCCTCTTCTGGGAGGGCAGCAGGGCTACCCTGAGCTG
AGGCAGCAGTGTGAGGCCAGGGCAGAGTGAGACCCAGCCCTCATCCCGAGCACC
TCCACATCCTCCACGTTCTGCTCATCATTCTCTGTCTCATCCATCATCATGTGTGT
CCACGACTGTCTCCATGGCCCCGCAAAAGGACTCTCAGGACCAAAGCTTTCATGT
AAACTGTGCACCAAGCAGGAAATGAAAATGTCTTGTGTTACCTGAAAACACTGT
GCACATCTGTGTCTTGTTTGGAATATTGTCCATTGTCCAATCCTATGTTTTTGTTC
AAAGCCAGCGTCCTCCTCTGTGACCAATGTCTTGATGCATGCACTGTTCCCCCTGT
GCAGCCGCTGAGCGAGGAGATGCTCCTTGGGCCCTTTGAGTGCAGTCCTGATCAG
AGCCGTGGTCCTTTGGGGTGAACTACCTTGGTTCCCCCACTGATCACAAAAACAT
GGTGGGTCCATGGGCAGAGCCCAAGGGAATTCGGTGTGCACCAGGGTTGACCCC
AGAGGATTGCTGCCCCATCAGTGCTCCCTCACATGTCAGTACCTTCAAACTAGGG
CCAAGCCCAGCACTGCTTGAGGAAAACAAGCATTCACAACTTGTTTTTGGTTTTT
AAAACCCAGTCCACAAAATAACCAATCCTGGACATGAAGATTCTTTCCCAATTCA
CATCTAACCTCATCTTCTTCACCATTTGGCAATGCCATCATCTCCTGCCTTCCTCC
TGGGCCCTCTCTGCTCTGCGTGTCACCTGTGCTTCGGGCCCTTCCCACAGGACATT
TCTCTAAGAGAACAATGTGCTATGTGAAGAGTAAGTCAACCTGCCTGACATTTGG
AGTGTTCCCCTTCCACTGAGGGCAGTCGATAGAGCTGTATTAAGCCACTTAAAAT
GTTCACTTTTGACAAAGGCAAGCACTTGTGGGTTTTTGTTTTGTTTTTCATTCAGT
CTTACGAATACTTTTGCCCTTTGATTAAAGACTCCAGTTAAAAAAAATTTTAATG
AAGAAAGTGGAAAACAAGGAAGTCAAAGCAAGGAAACTATGTAACATGTAGGA
AGTAGGAAGTAAATTATAGTGATGTAATCTTGAATTGTAACTGTTCTTGAATTTA
ATAATCTGTAGGGTAATTAGTAACATGTGTTAAGTATTTTCATAAGTATTTCAAA
TTGGAGCTTCATGGCAGAAGGCAAACCCATCAACAAAAATTGTCCCTTAAACAA
AAATTAAAATCCTCAATCCAGCTATGTTATATTGAAAAAATAGAGCCTGAGGGAT
CTTTACTAGTTATAAAGATACAGAACTCTTTCAAAACCTTTTGAAATTAACCTCTC
ACTATACCAGTATAATTGAGTTTTCAGTGGGGCAGTCATTATCCAGGTAATCCAA
GATATTTTAAAATCTGTCACGTAGAACTTGGATGTACCTGCCCCCAATCCATGAA
CCAAGACCATTGAATTCTTGGTTGAGGAAACAAACATGACCCTAAATCTTGACTA
CAGTCAGGAAAGGAATCATTTCTATTTCTCCTCCATGGGAGAAAATAGATAAGA
GTAGAAACTGCAGGGAAAATTATTTGCATAACAATTCCTCTACTAACAATCAGCT
CCTTCCTGGAGACTGCCCAGCTAAAGCAATATGCATTTAAATACAGTCTTCCATT
TGCAAGGGAAAAGTCTCTTGTAATCCGAATCTCTTTTTGCTTTCGAACTGCTAGTC
AAGTGCGTCCACGAGCTGTTTACTAGGGATCCCTCATCTGTCCCTCCGGGACCTG
GTGCTGCCTCTACCTGACACTCCCTTGGGCTCCCTGTAACCTCTTCAGAGGCCCTC
GCTGCCAGCTCTGTATCAGGACCCAGAGGAAGGGGCCAGAGGCTCGTTGACTGG
CTGTGTGTTGGGATTGAGTCTGTGCCACGTGTTTGTGCTGTGGTGTGTCCCCCTCT
GTCCAGGCACTGAGATACCAGCGAGGAGGCTCCAGAGGGCACTCTGCTTGTTATT
AGAGATTACCTCCTGAGAAAAAAGGTTCCGCTTGGAGCAGAGGGGCTGAATAGC
AGAAGGTTGCACCTCCCCCAACCTTAGATGTTCTAAGTCTTTCCATTGGATCTCAT
TGGACCCTTCCATGGTGTGATCGTCTGACTGGTGTTATCACCGTGGGCTCCCTGA
CTGGGAGTTGATCGCCTTTCCCAGGTGCTACACCCTTTTCCAGCTGGATGAGAAT
TTGAGTGCTCTGATCCCTCTACAGAGCTTCCCTGACTCATTCTGAAGGAGCCCCA
TTCCTGGGAAATATTCCCTAGAAACTTCCAAATCCCCTAAGCAGACCACTGATAA
AACCATGTAGAAAATTTGTTATTTTGCAACCTCGCTGGACTCTCAGTCTCTGAGC
AGTGAATGATTCAGTGTTAAATGTGATGAATACTGTATTTTGTATTGTTTCAATTGCATCT
CCCAGATAATGTGAAAATGGTCCAGGAGAAGGCCAATTCCTATACGCAG
CGTGCTTTAAAAAATAAATAAGAAACAACTCTTTGAGAAACAACAATTTCTACTT
TGAAGTCATACCAATGAAAAAATGTATATGCACTTATAATTTTCCTAATAAAGTTCTGTAC
TCAAATGTAGCCACCAA.

H. sapiens CXCL12, transcript variant 3, mRNA, (GI 76563932):

(SEQ ID NO.: 2)
GCACTTTCACTCTCCGTCAGCCGCATTGCCCGCTCGGCGTCCGGCC
CCCGACCCGCGCTCGTCCGCCCGCCCGCCCGCCCGCCCGCGCCATG
AACGCCAAGGTCGTGGTCGTGCTGGTCCTCGTGCTGACCGCGCTCT
GCCTCAGCGACGGGAAGCCCGTCAGCCTGAGCTACAGATGCCCATG
CCGATTCTTCGAAAGCCATGTTGCCAGAGCCAACGTCAAGCATCTC
AAAATTCTCAACACTCCAAACTGTGCCCTTCAGATTGTAGCCCGGC
TGAAGAACAACAACAGACAAGTGTGCATTGACCCGAAGCTAAAGTG
GATTCAGGAGTACCTGGAGAAAGCTTTAAACAAGGGGCGCAGAGAA
GAAAAAGTGGGGAAAAAAGAAAAGATAGGAAAAAAGAAGCGACAGA
AGAAGAGAAAGGCTGCCCAGAAAAGGAAAAACTAGTTATCTGCCAC
CTCGAGATGGA.

H. sapiens CXCL12, transcript variant 1, mRNA, (GI 76563931):

(SEQ ID NO.: 3)
GCACTTTCACTCTCCGTCAGCCGCATTGCCCGCTCGGCGTCCGGCCCCCGACCCGCGCTC
GTCCGCCCGCCCGCCCGCCCGCCCGCGCCATGAACGCCAAGGTCGTGGTCGTGC
TGGTCCTCGTGCTGACCGCGCTCTGCCTCAGCGACGGGAAGCCCGTCAGCCTGA
GCTACAGATGCCCATGCCGATTCTTCGAAAGCCATGTTGCCAGAGCCAACGTCAA
GCATCTCAAAATTCTCAACACTCCAAACTGTGCCCTTCAGATTGTAGCCCGG
CTGAAGAACAACAACAGACAAGTGTGCATTGACCCGAAGCTAAAGTGGATTCAG
GAGTACCTGGAGAAAGCTTTAAACAAGTAAGCACAACAGCCAAAAAGGACTTTCCGC
TAGACCCACTCGAGGAAAACTAAAACCTTGTGAGAGATGAAAGGGCAAAGA
CGTGGGGGAGGGGGCCTTAACCATGAGGACCAGGTGTGTGTGTGGGGTGGGCAC
ATTGATCTGGGATCGGGCCTGAGGTTTGCCAGCATTTAGACCCTGCATTTATAGC
ATACGGTATGATATTGCAGCTTATATTCATCCATGCCCTGTACCTGTGCACGTTGGAAC
TTTTATTACTGGGGTTTTTCTAAGAAAGAAATTGTATTATCAACAGCATTTTCAAG
CAGTTAGTTCCTTCATGATCATCACAATCATCATCATTCTCATTCTCATTTTT
TAAATCAACGAGTACTTCAAGATCTGAATTTGGCTTGTTTGGAGCATCTCCTCTG
CTCCCCTGGGGAGTCTGGGCACAGTCAGGTGGTGGCTTAACAGGGAGCTGGAAAAAG
TGTCCTTTCTTCAGACACTGAGGCTCCCGCAGCAGCGCCCCTCCCAAGAGGAAGG
CCTCTGTGGCACTCAGATACCGACTGGGGCTGGGCGCCGCCACTGCCTTCACCTC
CTCTTTCAACCTCAGTGATTGGCTCTGTGGGCTCCATGTAGAAGCCACTATTACT
GGGACTGTGCTCAGAGACCCCTCTCCCAGCTATTCCTACTCTCTCCCCGACTCC
GAGAGCATGCTTAATCTTGCTTCTGCTTCTCATTTCTGTAGCCTGATCAGCGCCGC
ACCAGCCGGGAAGAGGGTGATTGCTGGGGCTCGTGCCCTGCATCCCTCTCCTCCC
AGGGCCTGCCCCACAGCTCGGGCCCTCTGTGAGATCCGTCTTTGGCCTCCTCCAG
AATGGAGCTGGCCCTCTCCTGGGGATGTGTAATGGTCCCCCTGCTTACCCGCAAA
AGACAAGTCTTTACAGAATCAAATGCAATTTTAAATCTGAGAGCTCGCTTTGAGT
GACTGGGTTTTGTGATTGCCTCTGAAGCCTATGTATGCCATGGAGGCACTAACAA
ACTCTGAGGTTTCCGAAATCAGAAGCGAAAAAATCAGTGAATAAACCATCATCT
TGCCACTACCCCCTCCTGAAGCCACAGCAGGGTTTCAGGTTCCAATCAGAACTGT
TGGCAAGGTGACATTTCCATGCATAAATGCGATCCACAGAAGGTCCTGGTGGTAT
TTGTAACTTTTTGCAAGGCATTTTTTTATATATATTTTTGTGCACATTTTTTTTTAC
GTTTCTTTAGAAAACAAATGTATTTCAAAATATATTTATAGTCGAACAATTCATA
TATTTGAAGTGGAGCCATATGAATGTCAGTAGTTTATACTTCTCTATTATCTCAAACTA
CTGGCAATTTGTAAAGAAATATATATGATATATAAATGTGATTGCAGCTTTT
CAATGTTAGCCACAGTGTATTTTTTCACTTGTACTAAAATTGTATCAAATGTGACATTA
TATGCACTAGCAATAAAATGCTAATTGTTTCATGGTATAAACGTCCTACTGTAT
GTGGGAATTTATTTACCTGAAATAAAATTCATTAGTTGTTAGTGATGGAGCTTAAAA
AAAA.

In certain embodiments, a CXCL12 and/or CXCR4 antagonist is an RNAi-inducing entity that targets CXCR4. The following sequences may be used to design RNAi-inducing entities that target CXCR4, in accordance with the guidelines described herein:

H. sapiens CXCR4, transcript variant 2, mRNA (GI 56790928):

(SEQ ID NO.: 4)
AACTTCAGTTTGTTGGCTGCGGCAGCAGGTAGCAAAGTGACGCCGAGGGCCTGAGTGC
TCCAGTAGCCACCGCATCTGGAGAACCAGCGGTTACCATGGAGGGGATCAGTATA
TACACTTCAGATAACTACACCGAGGAAATGGGCTCAGGGGACTATGACT
CCATGAAGGAACCCTGTTTCCGTGAAGAAAATGCTAATTTCAATAAAATCTTCCT
GCCCACCATCTACTCCATCATCTTCTTAACTGGCATTGTGGGCAATGGATTGGTC
ATCCTGGTCATGGGTTACCAGAAGAAACTGAGAAGCATGACGGACAAGTACAGG
CTGCACCTGTCAGTGGCCGACCTCCTCTTTGTCATCACGCTTCCCTTCTGGGCAGT
TGATGCCGTGGCAAACTGGTACTTTGGGAACTTCCTATGCAAGGCAGTCCATGTC
ATCTACACAGTCAACCTCTACAGCAGTGTCCTCATCCTGGCCTTCATCAGTCTGG
ACCGCTACCTGGCCATCGTCCACGCCACCAACAGTCAGAGGCCAAGGAAGCTGT
TGGCTGAAAAGGTGGTCTATGTTGGCGTCTGGATCCCTGCCCTCCTGCTGACTATTCCC
GACTTCATCTTTGCCAACGTCAGTG
AGGCAGATGACAGATATATCTGTGACCGCTTCTACCCCAATGACTTGTGGGTGGT
TGTGTTCCAGTTTCAGCACATCATGGTTGGCCTTATCCTGCCTGGTATTGTCATCC
TGTCCTGCTATTGCATTATCATCTCCAAGCTGTCACACTCCAAGGGCCACCAGAA
GCGCAAGGCCCTCAAGACCACAGTCATCCTCATCCTGGCTTTCTTCGCCTGTTGG
CTGCCTTACTACATTGGGATCAGCATCGACTCCTTCATCCTCCTGGAAATCATCA
AGCAAGGGTGTGAGTTTGAGAACACTGTGCACAAGTGGATTTCCATCACCGAGG
CCCTAGCTTTCTTCCACTGTTGTCTGAACCCCATCCTCTATGCTTTCCTTGGAGCC
AAATTTAAAACCTCTGCCCAGCACGCACTCACCTCTGTGAGCAGAGGGTCCAGCC
TCAAGATCCTCTCCAAAGGAAAGCGAGGTGGACATTCATCTGTTTCCACTGAGTC
TGAGTCTTCAAGTTTTCACTCCAGCTAACACAGATGTAAAAGACTTTTTTTTATAC
GATAAATAACTTTTTTTTAAGTTACACATTTTTCAGATATAAAAGACTGACCAAT
ATTGTACAGTTTTTATTGCTTGTTGGATTTTTGTCTTGTGTTTCTTTAGTTTTTGTG
AAGTTTAATTGACTTATTTATATAAATTTTTTTTGTTTCATATTGATGTGTGTCTAG
GCAGGACCTGTGGCCAAGTTCTTAGTTGCTGTATGTCTCGTGGTAGGACTGTAGA
AAAGGGAACTGAACATTCCAGAGCGTGTAGTGAATCACGTAAAGCTAGAAATGA
TCCCCAGCTGTTTATGCATAGATAATCTCTCCATTCCCGTGGAACGTTTTTCCTGT
TCTTAAGACGTGATTTTGCTGTAGAAGATGGCACTTATAACCAAAGCCCAAAGTGGTA
TAGAAATGCTGGTTTTTCAGTTTTCAGGAGTGGGTTGATTTCAGCACCTACA
GTGTACAGTCTTGTATTAAGTTGTTAATAAAAGTACATGTTAAACTTAAAAAAAAAAAAA
AAAAA.

H. sapiens CXCR4, transcript variant 1, mRNA (GI 56790926):

(SEQ ID NO.: 5)
TTTTTTTTCTTCCCTCTAGTGGGCGGGGCAGAGGAGTTAGCCAAGATGTGACTTTGA
AACCCTCAGCGTCTCAGTGCCCTTTTGTTCTAAACAAAGAATTTTGTAATTGG
TTCTACCAAAGAAGGATATAATGAAGTCACTATGGGAAAAGATGGGGAGGAGAGTTG
TAGGATTCTACATTAATTCTCTTGTGCCCTTAGCCCACTACTTCAGAATTTCC
TGAAGAAAGCAAGCCTGAATTGGTTTTTTAAATTGCTTTAAAAATTTTTTTTAACT
GGGTTAATGCTTGCTGAATTGGAAGTGAATGTCCATTCCTTTGCCTCTTTTGCAGA
TATACACTTCAGATAACTACACCGAGGAAATGGGCTCAGGGGACTATGACTCCA
TGAAGGAACCCTGTTTCCGTGAAGAAAATGCTAATTTCAATAAAATCTTCCTGCC
CACCATCTACTCCATCATCTTCTTAACTGGCATTGTGGGCAATGGATTGGTCATCC
TGGTCATGGGTTACCAGAAGAAACTGAGAAGCATGACGGACAAGTACAGGCTGCACCT
GTCAGTGGCCGACCTCCTCTTTGTCATCACGCTTCCCTTCTGGGCAGTTGAT
GCCGTGGCAAACTGGTACTTTGGGAACTTCCTATGCAAGGCAGTCCATGTCATCT
ACACAGTCAACCTCTACAGCAGTGTCCTCATCCTGGCCTTCATCAGTCTGGACCG
CTACCTGGCCATCGTCCACGCCACCAACAGTCAGAGGCCAAGGAAGCTGTTGGC
TGAAAAGGTGGTCTATGTTGGCGTCTGGATCCCTGCCCTCCTGCTGACTATTCCC
GACTTCATCTTTGCCAACGTCAGTGAGGCAGATGACAGATATATCTGTGACCGCT
TCTACCCCAATGACTTGTGGGTGGTTGTGTTCCAGTTTCAGCACATCATGGTTGGC
CTTATCCTGCCTGGTATTGTCATCCTGTCCTGCTATTGCATTATCATCTCCAAGCT
GTCACACTCCAAGGGCCACCAGAAGCGCAAGGCCCTCAAGACCACAGTCATCCT
CATCCTGGCTTTCTTCGCCTGTTGGCTGCCTTACTACATTGGGATCAGCATCGACT
CCTTCATCCTCCTGGAAATCATCAAGCAAGGGTGTGAGTTTGAGAACACTGTGCA
CAAGTGGATTTCCATCACCGAGGCCCTAGCTTTCTTCCACTGTTGTCTGAACCCCA
TCCTCTATGCTTTCCTTGGAGCCAAATTTAAAACCTCTGCCCAGCACGCACTCACC
TCTGTGAGCAGAGGGTCCAGCCTCAAGATCCTCTCCAAAGGAAAGCGAGGTGGA
CATTCATCTGTTTCCACTGAGTCTGAGTCTTCAAGTTTTCACTCCAGCTAACACAG
ATGTAAAAGACTTTTTTTTATACGATAAATAACTTTTTTTTAAGTTACACATTTTT
CAGATATAAAAGACTGACCAATATTGTACAGTTTTTATTGCTTGTTGGATTTTTGT
CTTGTGTTTCTTTAGTTTTTGTGAAGTTTAATTGACTTATTTATATAAATTTTTTTT
GTTTCATATTGATGTGTGTCTAGGCAGGACCTGTGGCCAAGTTCTTAGTTGCTGTATGT
CTCGTGGTAGGACTGTAGAAAAGGGAACTGAACATTCCAGAGCGTGTAGTG
AATCACGTAAAGCTAGAAATGATCCCCAGCTGTTTATGCATAGATAATCTCTCCA
TTCCCGTGGAACGTTTTTCCTGTTCTTAAGACGTGATTTTGCTGTAGAAGATGGCA
CTTATAACCAAAGCCCAAAGTGGTATAGAAATGCTGGTTTTTCAGTTTTCAGGAG
TGGGTTGATTTCAGCACCTACAGTGTACAGTCTTGTATTAAGTTGTTAATAAAAGTACATG
TTAAACTTAAAAAAAAAAAAAAAAAA

In certain embodiments, CXCL12 and/or CXCR4 antagonist is an RNAi-inducing entity that targets one or more genes that have been shown to up-regulate CXCL12 and/or CXCR4 (e.g., HIF-1α, HIF-2α, Ets1, and NF-κB). See, for example, Ceradini et al., 2004, Nat. Med., 10:858; Liu et al., 2006, Cancer Biol. Ther., 5:1320; Maroni et al., 2007, Carcinogenesis, 28:267; and Helbig et al., 2003, J. Biol. Chem., 278:21631 (all of which are incorporated herein by reference).

The following sequences may be used to design RNAi-inducing entities that target genes that have been shown to upregulate CXCL12 and/or CXCR4, in accordance with the guidelines described herein:

H. sapiens hypoxia-inducible factor 1, alpha subunit (HIF1A), transcript variant 1, mRNA, (GI 31077212):

(SEQ ID NO.: 6)
GTGCTGCCTCGTCTGAGGGGACAGGAGGATCACCCTCTTCGTCGCTTCGGCCAGTGTG
TCGGGCTGGGCCCTGACAAGCCACCTGAGGAGAGGCTCGGAGCCGGGCCCG
GACCCCGGCGATTGCCGCCCGCTTCTCTCTAGTCTCACGAGGGGTTTCCCGCCTC
GCACCCCCACCTCTGGACTTGCCTTTCCTTCTCTTCTCCGCGTGTGGAGGGAGCCA
GCGCTTAGGCCGGAGCGAGCCTGGGGGCCGCCCGCCGTGAAGACATCGCGGGGA
CCGATTCACCATGGAGGGCGCCGGCGGCGCGAACGACAAGAAAAAGATAAGTTC
TGAACGTCGAAAAGAAAAGTCTCGAGATGCAGCCAGATCTCGGCGAAGTAAAGA
ATCTGAAGTTTTTTATGAGCTTGCTCATCAGTTGCCACTTCCACATAATGTGAGTT
CGCATCTTGATAAGGCCTCTGTGATGAGGCTTACCATCAGCTATTTGCGTGTGAG
GAAACTTCTGGATGCTGGTGATTTGGATATTGAAGATGACATGAAAGCACAGAT
GAATTGCTTTTATTTGAAAGCCTTGGATGGTTTTGTTATGGTTCTCACAGATGATG
GTGACATGATTTACATTTCTGATAATGTGAACAAATACATGGGATTAACTCAGTT
TGAACTAACTGGACACAGTGTGTTTGATTTTACTCATCCATGTGACCATGAGGAA
ATGAGAGAAATGCTTACACACAGAAATGGCCTTGTGAAAAAGGGTAAAGAACAA
AACACACAGCGAAGCTTTTTTCTCAGAATGAAGTGTACCCTAACTAGCCGAGGA
AGAACTATGAACATAAAGTCTGCAACATGGAAGGTATTGCACTGCACAGGCCAC
ATTCACGTATATGATACCAACAGTAACCAACCTCAGTGTGGGTATAAGAAACCA
CCTATGACCTGCTTGGTGCTGATTTGTGAACCCATTCCTCACCCATCAAATATTGA
AATTCCTTTAGATAGCAAGACTTTCCTCAGTCGACACAGCCTGGATATGAAATTT
TCTTATTGTGATGAAAGAATTACCGAATTGATGGGATATGAGCCAGAAGAACTTT
TAGGCCGCTCAATTTATGAATATTATCATGCTTTGGACTCTGATCATCTGACCAA
AACTCATCATGATATGTTTACTAAAGGACAAGTCACCACAGGACAGTACAGGAT
GCTTGCCAAAAGAGGTGGATATGTCTGGGTTGAAACTCAAGCAACTGTCATATAT
AACACCAAGAATTCTCAACCACAGTGCATTGTATGTGTGAATTACGTTGTGAGTG
GTATTATTCAGCACGACTTGATTTTCTCCCTTCAACAAACAGAATGTGTCCTTAAA
CCGGTTGAATCTTCAGATATGAAAATGACTCAGCTATTCACCAAAGTTGAATCAG
AAGATACAAGTAGCCTCTTTGACAAACTTAAGAAGGAACCTGATGCTTTAACTTT
GCTGGCCCCAGCCGCTGGAGACACAATCATATCTTTAGATTTTGGCAGCAACGAC
ACAGAAACTGATGACCAGCAACTTGAGGAAGTACCATTATATAATGATGTAATG
CTCCCCTCACCCAACGAAAAATTACAGAATATAAATTTGGCAATGTCTCCATTAC
CCACCGCTGAAACGCCAAAGCCACTTCGAAGTAGTGCTGACCCTGCACTCAATCA
AGAAGTTGCATTAAAATTAGAACCAAATCCAGAGTCACTGGAACTTTCTTTTACC
ATGCCCCAGATTCAGGATCAGACACCTAGTCCTTCCGATGGAAGCACTAGACAA
AGTTCACCTGAGCCTAATAGTCCCAGTGAATATTGTTTTTATGTGGATAGTGATA
TGGTCAATGAATTCAAGTTGGAATTGGTAGAAAAACTTTTTGCTGAAGACACAGA
AGCAAAGAACCCATTTTCTACTCAGGACACAGATTTAGACTTGGAGATGTTAGCT
CCCTATATCCCAATGGATGATGACTTCCAGTTACGTTCCTTCGATCAGTTGTCACC
ATTAGAAAGCAGTTCCGCAAGCCCTGAAAGCGCAAGTCCTCAAAGCACAGTTAC
AGTATTCCAGCAGACTCAAATACAAGAACCTACTGCTAATGCCACCACTACCACT
GCCACCACTGATGAATTAAAAACAGTGACAAAAGACCGTATGGAAGACATTAAA
ATATTGATTGCATCTCCATCTCCTACCCACATACATAAAGAAACTACTAGTGCCA
CATCATCACCATATAGAGATACTCAAAGTCGGACAGCCTCACCAAACAGAGCAG
GAAAAGGAGTCATAGAACAGACAGAAAAATCTCATCCAAGAAGCCCTAACGTGT
TATCTGTCGCTTTGAGTCAAAGAACTACAGTTCCTGAGGAAGAACTAAATCCAAA
GATACTAGCTTTGCAGAATGCTCAGAGAAAGCGAAAAATGGAACATGATGGTTC
ACTTTTTCAAGCAGTAGGAATTGGAACATTATTACAGCAGCCAGACGATCATGCA
GCTACTACATCACTTTCTTGGAAACGTGTAAAAGGATGCAAATCTAGTGAACAGA
ATGGAATGGAGCAAAAGACAATTATTTTAATACCCTCTGATTTAGCATGTAGACT
GCTGGGGCAATCAATGGATGAAAGTGGATTACCACAGCTGACCAGTTATGATTG
TGAAGTTAATGCTCCTATACAAGGCAGCAGAAACCTACTGCAGGGTGAAGAATT
ACTCAGAGCTTTGGATCAAGTTAACTGAGCTTTTTCTTAATTTCATTCCTTTTTTTG
GACACTGGTGGCTCACTACCTAAAGCAGTCTATTTATATTTTCTACATCTAATTTT
AGAAGCCTGGCTACAATACTGCACAAACTTGGTTAGTTCAATTTTTGATCCCCTTT
CTACTTAATTTACATTAATGCTCTTTTTTAGTATGTTCTTTAATGCTGGATCACAG
ACAGCTCATTTTCTCAGTTTTTTGGTATTTAAACCATTGCATTGCAGTAGCATCAT
TTTAAAAAATGCACCTTTTTATTTATTTATTTTTGGCTAGGGAGTTTATCCCTTTTT
CGAATTATTTTTAAGAAGATGCCAATATAATTTTTGTAAGAAGGCAGTAACCTTT
CATCATGATCATAGGCAGTTGAAAAATTTTTACACCTTTTTTTTCACATTTTACAT
AAATAATAATGCTTTGCCAGCAGTACGTGGTAGCCACAATTGCACAATATATTTT
CTTAAAAAATACCAGCAGTTACTCATGGAATATATTCTGCGTTTATAAAACTAGT
TTTTAAGAAGAAATTTTTTTTGGCCTATGAAATTGTTAAACCTGGAACATGACAT
TGTTAATCATATAATAATGATTCTTAAATGCTGTATGGTTTATTATTTAAATGGGT
AAAGCCATTTACATAATATAGAAAGATATGCATATATCTAGAAGGTATGTGGCAT
TTATTTGGATAAAATTCTCAATTCAGAGAAATCATCTGATGTTTCTATAGTCACTT
TGCCAGCTCAAAAGAAAACAATACCCTATGTAGTTGTGGAAGTTTATGCTAATAT
TGTGTAACTGATATTAAACCTAAATGTTCTGCCTACCCTGTTGGTATAAAGATATT
TTGAGCAGACTGTAAACAAGAAAAAAAAAATCATGCATTCTTAGCAAAATTGCC
TAGTATGTTAATTTGCTCAAAATACAATGTTTGATTTTATGCACTTTGTCGCTATT
AACATCCTTTTTTTCATGTAGATTTCAATAATTGAGTAATTTTAGAAGCATTATTT
TAGGAATATATAGTTGTCACAGTAAATATCTTGTTTTTTCTATGTACATTGTACAAAT
TTTTCATTCCTTTTGCTCTTTGTGGTTGGATCTAACACTAACTGTATTGTTTTGTTACATC
AAATAAACATCTTCTGTGGACCAGGAAAAAAAAAAAAAAAAAAA.

H. sapiens hypoxia-inducible factor 1, alpha subunit (HIF1A), transcript variant 2, mRNA, (GI 31077210):

(SEQ ID NO.: 7)
GTGCTGCCTCGTCTGAGGGGACAGGAGGATCACCCTCTTCGTCGCTTCGGCCAGTGTG
TCGGGCTGGGCCCTGACAAGCCACCTGAGGAGAGGCTCGGAGCCGGGCCCG
GACCCCGGCGATTGCCGCCCGCTTCTCTCTAGTCTCACGAGGGGTTTCCCGCCTCGCA
CCCCCACCTCTGGACTTGCCTTTCCTTCTCTTCTCCGCGTGTGGAGGGAGCCA
GCGCTTAGGCCGGAGCGAGCCTGGGGGCCGCCCGCCGTGAAGACATCGCGGGGA
CCGATTCACCATGGAGGGCGCCGGCGGCGCGAACGACAAGAAAAAGATAAGTTC
TGAACGTCGAAAAGAAAAGTCTCGAGATGCAGCCAGATCTCGGCGAAGTAAAGA
ATCTGAAGTTTTTTATGAGCTTGCTCATCAGTTGCCACTTCCACATAATGTGAGTT
CGCATCTTGATAAGGCCTCTGTGATGAGGCTTACCATCAGCTATTTGCGTGTGAG
GAAACTTCTGGATGCTGGTGATTTGGATATTGAAGATGACATGAAAGCACAGAT
GAATTGCTTTTATTTGAAAGCCTTGGATGGTTTTGTTATGGTTCTCACAGATGATG
GTGACATGATTTACATTTCTGATAATGTGAACAAATACATGGGATTAACTCAGTT
TGAACTAACTGGACACAGTGTGTTTGATTTTACTCATCCATGTGACCATGAGGAA
ATGAGAGAAATGCTTACACACAGAAATGGCCTTGTGAAAAAGGGTAAAGAACAA
AACACACAGCGAAGCTTTTTTCTCAGAATGAAGTGTACCCTAACTAGCCGAGGA
AGAACTATGAACATAAAGTCTGCAACATGGAAGGTATTGCACTGCACAGGCCAC
ATTCACGTATATGATACCAACAGTAACCAACCTCAGTGTGGGTATAAGAAACCA
CCTATGACCTGCTTGGTGCTGATTTGTGAACCCATTCCTCACCCATCAAATATTGA
AATTCCTTTAGATAGCAAGACTTTCCTCAGTCGACACAGCCTGGATATGAAATTT
TCTTATTGTGATGAAAGAATTACCGAATTGATGGGATATGAGCCAGAAGAACTTT
TAGGCCGCTCAATTTATGAATATTATCATGCTTTGGACTCTGATCATCTGACCAA
AACTCATCATGATATGTTTACTAAAGGACAAGTCACCACAGGACAGTACAGGAT
GCTTGCCAAAAGAGGTGGATATGTCTGGGTTGAAACTCAAGCAACTGTCATATAT
AACACCAAGAATTCTCAACCACAGTGCATTGTATGTGTGAATTACGTTGTGAGTG
GTATTATTCAGCACGACTTGATTTTCTCCCTTCAACAAACAGAATGTGTCCTTAAA
CCGGTTGAATCTTCAGATATGAAAATGACTCAGCTATTCACCAAAGTTGAATCAG
AAGATACAAGTAGCCTCTTTGACAAACTTAAGAAGGAACCTGATGCTTTAACTTT
GCTGGCCCCAGCCGCTGGAGACACAATCATATCTTTAGATTTTGGCAGCAACGAC
ACAGAAACTGATGACCAGCAACTTGAGGAAGTACCATTATATAATGATGTAATG
CTCCCCTCACCCAACGAAAAATTACAGAATATAAATTTGGCAATGTCTCCATTAC
CCACCGCTGAAACGCCAAAGCCACTTCGAAGTAGTGCTGACCCTGCACTCAATCA
AGAAGTTGCATTAAAATTAGAACCAAATCCAGAGTCACTGGAACTTTCTTTTACC
ATGCCCCAGATTCAGGATCAGACACCTAGTCCTTCCGATGGAAGCACTAGACAA
AGTTCACCTGAGCCTAATAGTCCCAGTGAATATTGTTTTTATGTGGATAGTGATA
TGGTCAATGAATTCAAGTTGGAATTGGTAGAAAAACTTTTTGCTGAAGACACAGA
AGCAAAGAACCCATTTTCTACTCAGGACACAGATTTAGACTTGGAGATGTTAGCT
CCCTATATCCCAATGGATGATGACTTCCAGTTACGTTCCTTCGATCAGTTGTCACC
ATTAGAAAGCAGTTCCGCAAGCCCTGAAAGCGCAAGTCCTCAAAGCACAGTTAC
AGTATTCCAGCAGACTCAAATACAAGAACCTACTGCTAATGCCACCACTACCACT
GCCACCACTGATGAATTAAAAACAGTGACAAAAGACCGTATGGAAGACATTAAA
ATATTGATTGCATCTCCATCTCCTACCCACATACATAAAGAAACTACTAGTGCCA
CATCATCACCATATAGAGATACTCAAAGTCGGACAGCCTCACCAAACAGAGCAG
GAAAAGGAGTCATAGAACAGACAGAAAAATCTCATCCAAGAAGCCCTAACGTGT
TATCTGTCGCTTTGAGTCAAAGAACTACAGTTCCTGAGGAAGAACTAAATCCAAA
GATACTAGCTTTGCAGAATGCTCAGAGAAAGCGAAAAATGGAACATGATGGTTC
ACTTTTTCAAGCAGTAGGAATTATTTAGCATGTAGACTGCTGGGGCAATCAATGG
ATGAAAGTGGATTACCACAGCTGACCAGTTATGATTGTGAAGTTAATGCTCCTAT
ACAAGGCAGCAGAAACCTACTGCAGGGTGAAGAATTACTCAGAGCTTTGGATCA
AGTTAACTGAGCTTTTTCTTAATTTCATTCCTTTTTTTGGACACTGGTGGCTCACT
ACCTAAAGCAGTCTATTTATATTTTCTACATCTAATTTTAGAAGCCTGGCTACAAT
ACTGCACAAACTTGGTTAGTTCAATTTTTGATCCCCTTTCTACTTAATTTACATTA
ATGCTCTTTTTTAGTATGTTCTTTAATGCTGGATCACAGACAGCTCATTTTCTCAG
TTTTTTGGTATTTAAACCATTGCATTGCAGTAGCATCATTTTAAAAAATGCACCTT
TTTATTTATTTATTTTTGGCTAGGGAGTTTATCCCTTTTTCGAATTATTTTTAAGAA
GATGCCAATATAATTTTTGTAAGAAGGCAGTAACCTTTCATCATGATCATAGGCA
GTTGAAAAATTTTTACACCTTTTTTTTCACATTTTACATAAATAATAATGCTTTGC
CAGCAGTACGTGGTAGCCACAATTGCACAATATATTTTCTTAAAAAATACCAGCA
GTTACTCATGGAATATATTCTGCGTTTATAAAACTAGTTTTTAAGAAGAAATTTTT
TTTGGCCTATGAAATTGTTAAACCTGGAACATGACATTGTTAATCATATAATAAT
GATTCTTAAATGCTGTATGGTTTATTATTTAAATGGGTAAAGCCATTTACATAATA
TAGAAAGATATGCATATATCTAGAAGGTATGTGGCATTTATTTGGATAAAATTCT
CAATTCAGAGAAATCATCTGATGTTTCTATAGTCACTTTGCCAGCTCAAAAGAAA
ACAATACCCTATGTAGTTGTGGAAGTTTATGCTAATATTGTGTAACTGATATTAA
ACCTAAATGTTCTGCCTACCCTGTTGGTATAAAGATATTTTGAGCAGACTGTAAA
CAAGAAAAAAAAAATCATGCATTCTTAGCAAAATTGCCTAGTATGTTAATTTGCT
CAAAATACAATGTTTGATTTTATGCACTTTGTCGCTATTAACATCCTTTTTTTCAT
GTAGATTTCAATAATTGAGTAATTTTAGAAGCATTATTTTAGGAATATATAGTTG
TCACAGTAAATATCTTGTTTTTTCTATGTACATTGTACAAATTTTTCATTCCTTTTG
CTCTTTGTGGTTGGATCTAACACTAACTGTATTGTTTTGTTACATCAAATAAACATCTTCT
GTGGACCAGG.

H. sapiens endothelial PAS domain protein 1 (EPAS1), mRNA, (GI 41327154):

(SEQ ID NO.: 8)
GCCACACGGGTCCGGTGCCCGCTGCGCTTCCGCCCCAGCGCTCCTGAGGCGGCCGTACA
ATCCTCGGCAGTGTCCTGAGACTGTATGGTCAGCTCAGCCCGGCCTCCGAC
TCCTTCCGACTCCCAGCATTCGAGCCACTTTTTTTTTTCTTTGAAAACTCAGAAAA
GTGACTCCTTTTCCAGGGAAAAAGGAACTTGGGTTCCCTTCTCTCCGTCCTCTTTT
CGGGTCTGACAGCCTCCACCCACTCCTTCCCCGGACCCCGCCTCCGCGCGCAGGT
TCCTCCCAGTCACCTTTCTCCACCCCCGCCCCCGCACCTAGCCCGCCGCGCGCCA
CCTTCCACCTGACTGCGCGGGGCGCTCGGGACCTGCGCGCACCTCGGACCTTCAC
CACCCGCCCGGGCCGCGGGGAGCGGACGAGGGCCACAGCCCCCCACCCGCCAGG
GAGCCCAGGTGCTCGGCGTCTGAACGTCTCAAAGGGCCACAGCGACAATGACAG
CTGACAAGGAGAAGAAAAGGAGTAGCTCGGAGAGGAGGAAGGAGAAGTCCCGG
GATGCTGCGCGGTGCCGGCGGAGCAAGGAGACGGAGGTGTTCTATGAGCTGGCC
CATGAGCTGCCTCTGCCCCACAGTGTGAGCTCCCATCTGGACAAGGCCTCCATCA
TGCGACTGGCAATCAGCTTCCTGCGAACACACAAGCTCCTCTCCTCAGTTTGCTC
TGAAAACGAGTCCGAAGCCGAAGCTGACCAGCAGATGGACAACTTGTACCTGAA
AGCCTTGGAGGGTTTCATTGCCGTGGTGACCCAAGATGGCGACATGATCTTTCTG
TCAGAAAACATCAGCAAGTTCATGGGACTTACACAGGTGGAGCTAACAGGACAT
AGTATCTTTGACTTCACTCATCCCTGCGACCATGAGGAGATTCGTGAGAACCTGA
GTCTCAAAAATGGCTCTGGTTTTGGGAAAAAAAGCAAAGACATGTCCACAGAGC
GGGACTTCTTCATGAGGATGAAGTGCACGGTCACCAACAGAGGCCGTACTGTCA
ACCTCAAGTCAGCCACCTGGAAGGTCTTGCACTGCACGGGCCAGGTGAAAGTCT
ACAACAACTGCCCTCCTCACAATAGTCTGTGTGGCTACAAGGAGCCCCTGCTGTC
CTGCCTCATCATCATGTGTGAACCAATCCAGCACCCATCCCACATGGACATCCCC
CTGGATAGCAAGACCTTCCTGAGCCGCCACAGCATGGACATGAAGTTCACCTACT
GTGATGACAGAATCACAGAACTGATTGGTTACCACCCTGAGGAGCTGCTTGGCC
GCTCAGCCTATGAATTCTACCATGCGCTAGACTCCGAGAACATGACCAAGAGTCA
CCAGAACTTGTGCACCAAGGGTCAGGTAGTAAGTGGCCAGTACCGGATGCTCGC
AAAGCATGGGGGCTACGTGTGGCTGGAGACCCAGGGGACGGTCATCTACAACCC
TCGCAACCTGCAGCCCCAGTGCATCATGTGTGTCAACTACGTCCTGAGTGAGATT
GAGAAGAATGACGTGGTGTTCTCCATGGACCAGACTGAATCCCTGTTCAAGCCCC
ACCTGATGGCCATGAACAGCATCTTTGATAGCAGTGGCAAGGGGGCTGTGTCTG
AGAAGAGTAACTTCCTATTCACCAAGCTAAAGGAGGAGCCCGAGGAGCTGGCCC
AGCTGGCTCCCACCCCAGGAGACGCCATCATCTCTCTGGATTTCGGGAATCAGAA
CTTCGAGGAGTCCTCAGCCTATGGCAAGGCCATCCTGCCCCCGAGCCAGCCATGG
GCCACGGAGTTGAGGAGCCACAGCACCCAGAGCGAGGCTGGGAGCCTGCCTGCC
TTCACCGTGCCCCAGGCAGCTGCCCCGGGCAGCACCACCCCCAGTGCCACCAGC
AGCAGCAGCAGCTGCTCCACGCCCAATAGCCCTGAAGACTATTACACATCTTTGG
ATAACGACCTGAAGATTGAAGTGATTGAGAAGCTCTTCGCCATGGACACAGAGG
CCAAGGACCAATGCAGTACCCAGACGGATTTCAATGAGCTGGACTTGGAGACAC
TGGCACCCTATATCCCCATGGACGGGGAAGACTTCCAGCTAAGCCCCATCTGCCC
CGAGGAGCGGCTCTTGGCGGAGAACCCACAGTCCACCCCCCAGCACTGCTTCAG
TGCCATGACAAACATCTTCCAGCCACTGGCCCCTGTAGCCCCGCACAGTCCCTTC
CTCCTGGACAAGTTTCAGCAGCAGCTGGAGAGCAAGAAGACAGAGCCCGAGCAC
CGGCCCATGTCCTCCATCTTCTTTGATGCCGGAAGCAAAGCATCCCTGCCACCGT
GCTGTGGCCAGGCCAGCACCCCTCTCTCTTCCATGGGGGGCAGATCCAATACCCA
GTGGCCCCCAGATCCACCATTACATTTTGGGCCCACAAAGTGGGCCGTCGGGGAT
CAGCGCACAGAGTTCTTGGGAGCAGCGCCGTTGGGGCCCCCTGTCTCTCCACCCC
ATGTCTCCACCTTCAAGACAAGGTCTGCAAAGGGTTTTGGGGCTCGAGGCCCAGA
CGTGCTGAGTCCGGCCATGGTAGCCCTCTCCAACAAGCTGAAGCTGAAGCGACA
GCTGGAGTATGAAGAGCAAGCCTTCCAGGACCTGAGCGGGGGGGACCCACCTGG
TGGCAGCACCTCACATTTGATGTGGAAACGGATGAAGAACCTCAGGGGTGGGAG
CTGCCCTTTGATGCCGGACAAGCCACTGAGCGCAAATGTACCCAATGATAAGTTC
ACCCAAAACCCCATGAGGGGCCTGGGCCATCCCCTGAGACATCTGCCGCTGCCA
CAGCCTCCATCTGCCATCAGTCCCGGGGAGAACAGCAAGAGCAGGTTCCCCCCA
CAGTGCTACGCCACCCAGTACCAGGACTACAGCCTGTCGTCAGCCCACAAGGTGT
CAGGCATGGCAAGCCGGCTGCTCGGGCCCTCATTTGAGTCCTACCTGCTGCCCGA
ACTGACCAGATATGACTGTGAGGTGAACGTGCCCGTGCTGGGAAGCTCCACGCT
CCTGCAAGGAGGGGACCTCCTCAGAGCCCTGGACCAGGCCACCTGAGCCAGGCC
TTCTACCTGGGCAGCACCTCTGCCGACGCCGTCCCACCAGCTTCACTCTCTCCGTC
TGTTTTTGCAACTAGGTATTTCTAACGCCAGCACACTATTTACAAGATGGACTTA
CCTGGCAGACTTGCCCAGGTCACCAAGCAGTGGCCTTTTTCTGAGATGCTCACTT
TATTATCCCTATTTTTAAAGTACACAATTGTTTTACCTGTTCTGAAATGTTCTTAA
ATTTTGTAGGATTTTTTTCCTCCCCACCTTCAATGACTTCTAATTTATATTATCCAT
AGGTTTCTCTCCCTCCTTCTCCTTCTCACACACAACTGTCCATACTAACAAGTTTG
GTGCATGTCTGTTCTTCTGTAGGGAGAAGCTTTAGCTTCATTTTACTAAAAAGATT
CCTCGTTATTGTTGTTGCCAAAGAGAAACAAAAATGATTTTGCTTTCCAAGCTTG
GTTTGTGGCGTCTCCCTCGCAGAGCCCTTCTCGTTTCTTTTTTAAACTAATCACCA
TATTGTAAATTTCAGGGTTTTTTTTTTTTTGTTTAAGCTGACTCTTTGCTCTAATTT
TGGAAAAAAAGAAATGTGAAGGGTCAACTCCAACGTATGTGGTTATCTGTGAAAGT
TGCACAGCGTGGCTTTTCCTAAACTGGTGTTTTTCCCCCGCATTTGGTGGATTT
TTTATTATTATTCAAAAACATAACTGAGTTTTTTAAAAGAGGAGAAAATTTATAT
CTGGGTTAAGTGTTTATCATATATATGGGTACTTTGTAATATCTAAAAACTTAGA
AACGGAAATGGAATCCTGCTCACAAAATCACTTTAAGATCTTTTCGAAGCTGTTA
ATTTTTCTTAGTGTTGTGGACACTGCAGACTTGTCCAGTGCTCCCACGGCCTGTAC
GGACACTGTGGAAGGCCTCCCTCTGTCGGCTTTTTGCCATCTGTGATATGCCATA
GGTGTGACAATCCGAGCAGTGGAGTCATTCAGCGGGAGCACTGCGCGCTATCCC
CTCACATTCTCTATGTACTATGTATGTATGTATTATTATTATTGCTGCCAAGAGGG
TCTGATGGCACGTTGTGGGGTCGGGGGGTGGGGCGGGGAAGTGCTCTAACTTTTC
TTAAGGTTTTGTTGCTAGCCCTTCAAGTGCACTGAGCTATGTGACTCGGATGGTCT
TTCACACGGCACATTTGGACATTTCCAGAACTACCATGAGATGGTTTAGACGGGA
ATTCATGCAAATGAGGGGTCAAAAATGGTATAGTGACCCCGTCCACGTCCTCCAA
GCTCACGACCTTGGAGCCCCGTGGAGCTGGACTGAGGAGGAGGCTGCACAGCGG
GAGAGCAGCTGGTCCAGACCAGCCCTGCAGCCCCCACTCAGCCGGCAGCCAGAT
GGCCCCGCAAGGCCTCCAGGGATGGCCCCTAGCCACAGGCCCTGGCTGAGGTCT
CTGGGTCGGTCAGTGACATGTAGGTAGGAAGCACTGAAAATAGTGTTCCCAGAG
CACTTTGCAACTCCCTGGGTAAGAGGGACGACACCTCTGGTTTTTCAATACCAAT
TACATGGAACTTTTCTGTAATGGGTACAATGAAGAAGTTTCTAAAAACACACACA
AAGCACATTGGGCCAACTATTTAGTAAGCCCGGATAGACTTATTGCCAAAAACA
AAAAATAGCTTTCAAAAGAAATTTAAGTTCTATGAGAAATTCCTTAGTCATGGTG
TTGCGTAAATCATATTTTAGCTGCACGGCATTACCCCACACAGGGTGGCAGAACT
TGAAGGGTTACTGACGTGTAAATGCTGGTATTTGATTTCCTGTGTGTGTTGCCCTG
GCATTAAGGGCATTTTACCCTTGCAGTTTTACTAAAACACTGAAAAATATTCCAA
GCTTCATATTAACCCTACCTGTCAACGTAACGATTTCATGAACGTTATTATATTGT
CGAATTCCTACTGACAACATTATAACTGTATGGGAGCTTAACTTTATAAGGAAAT
GTATTTTGACACTGGTATCTTATTAAAGTATTCTGATCCTAAAAAAAAAAAAAAAAAAAA
AAAAAAAAAAAAAAAAAAAAAAAAAAAAA.

H. sapiens v-cts erythroblastosis virus E26 oncogene homolog 1 (avian) (ETS1), mRNA, (GI 41393580):

(SEQ ID NO.: 9)
CGGGCGAGGGCCGGGCAGGAGGAGCGGGCGCGGCGCGGGCGAGGCTGGGACCCGAGC
GCGCTCACTTCGCCGCAAAGTGCCAACTTCCCCTGGAGTGCCGGGCGCGCAC
CGTCCGGGCGCGGGGGAAAGAAAGGCAGCGGGAATTTGAGATTTTTGGGAAG
AAAGTCGGATTTCCCCCGTCCCCTTCCCCCTGTTACTAATCCTCATTAAAAAGAA
AAACAACAGTAACTGCAAACTTGCTACCATCCCGTACGTCCCCCACTCCTGGCAC
CATGAAGGCGGCCGTCGATCTCAAGCCGACTCTCACCATCATCAAGACGGAAAA
AGTCGATCTGGAGCTTTTCCCCTCCCCGGATATGGAATGTGCAGATGTCCCACTA
TTAACTCCAAGCAGCAAAGAAATGATGTCTCAAGCATTAAAAGCTACTTTCAGTG
GTTTCACTAAAGAACAGCAACGACTGGGGATCCCAAAAGACCCCCGGCAGTGGA
CAGAAACCCATGTTCGGGACTGGGTGATGTGGGCTGTGAATGAATTCAGCCTGA
AAGGTGTAGACTTCCAGAAGTTCTGTATGAATGGAGCAGCCCTCTGCGCCCTGGG
TAAAGACTGCTTTCTCGAGCTGGCCCCAGACTTTGTTGGGGACATCTTATGGGAA
CATCTAGAGATCCTGCAGAAAGAGGATGTGAAACCATATCAAGTTAATGGAGTC
AACCCAGCCTATCCAGAATCCCGCTATACCTCGGATTACTTCATTAGCTATGGTA
TTGAGCATGCCCAGTGTGTTCCACCATCGGAGTTCTCAGAGCCCAGCTTCATCAC
AGAGTCCTATCAGACGCTCCATCCCATCAGCTCGGAAGAGCTCCTCTCCCTCAAG
TATGAGAATGACTACCCCTCGGTCATTCTCCGAGACCCTCTCCAGACAGACACCT
TGCAGAATGACTACTTTGCTATCAAACAAGAAGTCGTCACCCCAGACAACATGTG
CATGGGGAGGACCAGTCGTGGTAAACTCGGGGGCCAGGACTCTTTTGAAAGCAT
AGAGAGCTACGATAGTTGTGATCGCCTCACCCAGTCCTGGAGCAGCCAGTCATCT
TTCAACAGCCTGCAGCGTGTTCCCTCCTATGACAGCTTCGACTCAGAGGACTATC
CGGCTGCCCTGCCCAACCACAAGCCCAAGGGCACCTTCAAGGACTATGTGCGGG
ACCGTGCTGACCTCAATAAGGACAAGCCTGTCATTCCTGCTGCTGCCCTAGCTGG
CTACACAGGCAGTGGACCAATCCAGCTATGGCAGTTTCTTCTGGAATTACTCACT
GATAAATCCTGTCAGTCTTTTATCAGCTGGACAGGAGATGGCTGGGAATTCAAAC
TTTCTGACCCAGATGAGGTGGCCAGGAGATGGGGAAAGAGGAAAAACAAACCTA
AGATGAATTATGAGAAACTGAGCCGTGGCCTACGCTACTATTACGACAAAAACA
TCATCCACAAGACAGCGGGGAAACGCTACGTGTACCGCTTTGTGTGTGACCTGCA
GAGCCTGCTGGGGTACACCCCTGAGGAGCTGCACGCCATGCTGGACGTCAAGCC
AGATGCCGACGAGTGATGGCACTGAAGGGGCTGGGGAAACCCTGCTGAGACCTT
CCAAGGACAGCCGTGTTGGTTGGACTCTGAATTTTGAATTGTTATTCTATTTTTTA
TTTTCCAGAACTCATTTTTTACCTTCAGGGGTGGGAGCTAAGTCAGTTGCAGCTGT
AATCAATTGTGCGCAGTTGGGAAAGGAAAGCCAGGACTTGTGGGGTGGGTGGGA
CCAGAAATTCTTGAGCAAATTTTCAGGAGAGGGAGAAGGGCCTTCTCAGAAGCT
TGAAGGCTCTGGCTTAACAGAGAAAGAGACTAATGTGTCCAATCATTTTTAAAAA
TCATCCATGAAAAAGTGTCTTGAGTTGTGGACCCATTAGCAAGTGACATTGTCAC
ATCAGAACTCATGAAACTGATGTAAGGCAATTAATTTGCTTCTGTTTTTAGGTCT
GGGAGGGCAAAAAAGAGGTGGGTGGGATGAAACATGTTTTGGGGGGGGATGCA
CTGAAAATCTGAGAACTATTTACCTATCACTCTAGTTTTGAAGCAAAGATGGACT
TCAGTGGGGAGGGGCCAAAACCGTTGTTGTGTTAAAATTTATTTTATTAAATTTT
GTGCCAGTATTTTTTTTCTTAAAAATCGTCTTAAGCTCTAAGGTGGTCTCAGTATT
GCAATATCATGTAAGTTTGTTTTTATTTGCCGGCTGAGGATTCTGTCACAATGAA
AGAAAACTGTTTATATAGACCCCATTGGAAAAGCAAAACGCTCTCACTGAGATC
AGGGATCCCAAATTCATGGGACTTATATAAGAAGGACAATTAATGCTGATTTGG
GTACAGGGGAATTATGTGTGTGAATGTCATCTACAATTAAAAAAAATTAGCACAT
CCCTTTACTTACTTGTTATCAGTGGATTCTCGGGGTTTGGACTTAATGTTGAGCTA
AGAAGCATTAAGTCTTTGAACTGAATGTATTTTGCATCCCTGGTTTTGGACGACA
GTAAACGTAGGAGCACTGTTGAAGTCCTGGAAGGGAGATCGAAGGAGGAAGATT
GACTTGGTTCTTTCTTAGTCCTATATCTGTAGCATAGATGACTTGGAATAAAAGCT
GTATGCATGGGCATTACCCCTCAGGTCCTAAGAAATAAGTCCTGAATGCATGTCG
TTCCAAACTAACACTCTGTAATTTTTCTTTTATGTCTTATTTTCCAAGAGTCCTCCA
TTTTTTGCACCCCCTCACCGCCAACTCTGTTATTCAGTAGAGAGAAGTGTACGGC
TTTCTGATTGGTGAGTGAAAAAGTAACTTGAGACACGACCTAAGTTGAAGAGTTT
AGACTTGCTGAGTTTTAGAAGTGATGGAAATTAAGAGAGCATTTCAATAAAATGT
GACTTGGCTGTCTTTGGAAGAGAAGTGCAAGGCTTTCCTTTGAAGAATTTAAATT
AGTCCGGTAGGATGTCAGGTGAGACTGTGTATGCAAAATGAATGGCACAGGTGA
TGCCAGGGCCTCTTGCTTGGGTCTGATGTCTTGGCACAGGGTAAGTGAAGGTTAA
TTCCAGAAGAGAGGAATGACTTGAAGGCAAAGGAAACTAAGGAAGGAGGTTCA
GTGAGGAAAATAAGGTTGTCCATGAGATTTGAATAGATTTTTAGTTCCCCCAAGGTTT
AAATACAAACATAGTCAAGCAAGGTAGTCATCTTTCTGCTGGTTGTGAGGGG
GAATCTGAAAATGGAGTTTTAGAGGAAAAGTCAACATCTAACTAGTGAGGAAAA
GTGCCTAATACAATTAGAATCTCCCTCACTCTATAGTTGCCCAGTTGAAAGGATA
AGGAGGAGGGGTGGCTTTTATGGACTTCCATGAGAGAAGGAAAGAAATATTTCA
GGTAAGCTTCTCAGGGCTGGCCCTTTTTGGGATTTGGATGAGAAATTGGAAGTAC
TAACTACTTTCTAGCATATCTTTAAGAAAATTGATTGTTATTTACTCCCAGATCCT
CTTGCAGACCCAGAATTATCAGGAACATAGCTCTGTGATTCATGAGTGTCCCCAT
ACTGATGAATTGGAGCATCCATATGGAAAGCAAAGGCAGAATTATCCCAGCTGT
ATTATTTTGATCTTTTGGATGCAGGTGCCTTAATGAAGCTCTCAAAATATTTTAGG
AGCTGCTCAGGGAGTGTTGGGTGGAACTGTTTGGACTACATTGTTTTCTCTTAGA
TTATGTGATTTTTGTTGGGCACTGGCAAAAGGTGTGTGTGTGAATGTGTGCATGT
GTGTGAATGTTGTGTGTGTGTGTGTGTGTGTGTGTGTGTGTGTGTGTGTTTGCAGA
CATGCAAAACTGCAGCTGAAATAATACCTTAGATTTCTAGGTAAGTCTTTCCACA
TTTCAATAATGGGTAAGAGTAGAACCAGGGCCGGGTATCAATTATTGCTTGCTGT
TTGCAACCAGGCATAAAATCACTTTCTCAAATCATCCACCGTTCCTATTAAATTTA
TGCCGGAAACTCTCCTTCTGTGAGTATAACTCCTGCAGTTCCTATAGCAGATAAG
ATATAAGAAAGTGCCTCCTAGTGCTCCTCCGCCCGCTTGTTTGCTAAAATTCCCTT
TCTCTCTAAGTCCACCATTTTCAAGATTTGTAGATAGTGTATTAGTTAAGACAGCT
TTGTCGATCTGGCCAGATGTTTTTTCTCCTTTGTCCAAAGGCCAGAGACCATCCCA
GGAAGAGTGGTGGGTGGTTTATACACTGGAAATGTTGCGTTTATGCTTTTTAAAA
ACACACGTTAACTTCAGAGGAAGGATGGGCAAATCTGGTCTAGCTGGGTGAAAC
CCTTATTTTCCCAGAGATGCCTTAACCTTTGTTGGTTTTGGCTTTAGGGTTCAGAG
TCACTTTTGTTCCCTTCTCCATTCTGGAGAGGGACTTCCCCTACATAGAGCCCTGA
TTTTTGTGGCTGTGGGGATTGGAGGTAGCATTCAAAGATCAGATGTGCTTTTCCT
CACTTTGGAGATGAACACTCTGGGTTTTACAGCATTAACCTGCCTAACCTTCATG
GTGAGAAATACACCATCTCTCTTCTAGTCATGCTGTGCATGCCGCTTACTCTGTTG
GGGTCTATATAAATTTGTTGAACTCTTACCTACATTCCAAAGAAGTTTCAAGGAA
CCATAAATATATGTATACATATACATATATAAAATATATATATTAAAATAAAATT
ATCAGGAATACTGCCTCAGTTATTGAACTTTTTTTTTTAAGAATACTTTTTTTTTA
AGCTGAGAAGTATAGGGATGAAAAAGATGTTATATTGTGTTTGACTATTTTCCAA
CTTGTATTTTCATATAATTTATATTTTTTAAAAGCTGAAAATTTAGAAGCAAGATG
AAAAAAAGGAAAAGCAGGTGCTTTTTAAAAATCAGAACTGAGGTAGCTTAGAGA
TGTAGCGATGTAAGTGTCGATGTTTTTTTAAAAAAAAATGCAAAAAAATTCTTAT
GGCGGAGTTTTTTGTTTGTTTATTTTAGTAGCTGATGCTGGCACATCATTTTGCTG
GAGAGTTTTTTATATACTGTAGCCTGATTTCATATTGTATTTTAAACTGTGTGAAA
TTAAAAACAAAGAATTTCATTCATAAAAAAAAAAAAAAAAAAAAAAAAAAAAAAAAAAAAA
AAAAA.

H. sapiens nuclear factor of kappa light polypeptide gene enhancer in B-cells 1 (p105) (NF-κB1), mRNA, (GI 34577121):

(SEQ ID NO.: 10)
GTGAGAGAGTGAGCGAGACAGAAAGAGAGAGAAGTGCACCAGCGAGCCGGGGC
AGGAAGAGGAGGTTTCGCCACCGGAGCGGCCCGGCGACGCGCTGACAGCTTCCC
CTGCCCTTCCCGTCGGTCGGGCCGCCAGCCGCCGCAGCCCTCGGCCTGCACGCAG
CCACCGGCCCCGCTCCCGGAGCCCAGCGCCGCCGAGGCCGCAGCCGCCCGGCCA
GTAAGGCGGCGCCGCCGCCCGGCCACCGCGCGCCCTGCGCTTCCCTCCGCCCGCG
CTGCGGCCATGGCGCGGCGCTGACTGGCCTGGCCCGGCCCCGCCGCGCTCCCGCT
CGCCCCGACCCGCACTCGGGCCCGCCCGGGCTCCGGCCTGCCGCCGCCTCTTCCT
TCTCCAGCCGGCAGGCCCGCGCCGCTTAGGAGGGAGAGCCCACCCGCGCCAGGA
GGCCGAACGCGGACTCGCCACCCGGCTTCAGAATGGCAGAAGATGATCCATATT
TGGGAAGGCCTGAACAAATGTTTCATTTGGATCCTTCTTTGACTCATACAATATTT
AATCCAGAAGTATTTCAACCACAGATGGCACTGCCAACAGCAGATGGCCCATAC
CTTCAAATATTAGAGCAACCTAAACAGAGAGGATTTCGTTTCCGTTATGTATGTG
AAGGCCCATCCCATGGTGGACTACCTGGTGCCTCTAGTGAAAAGAACAAGAAGT
CTTACCCTCAGGTCAAAATCTGCAACTATGTGGGACCAGCAAAGGTTATTGTTCA
GTTGGTCACAAATGGAAAAAATATCCACCTGCATGCCCACAGCCTGGTGGGAAA
ACACTGTGAGGATGGGATCTGCACTGTAACTGCTGGACCCAAGGACATGGTGGT
CGGCTTCGCAAACCTGGGTATACTTCATGTGACAAAGAAAAAAGTATTTGAAAC
ACTGGAAGCACGAATGACAGAGGCGTGTATAAGGGGCTATAATCCTGGACTCTT
GGTGCACCCTGACCTTGCCTATTTGCAAGCAGAAGGTGGAGGGGACCGGCAGCT
GGGAGATCGGGAAAAAGAGCTAATCCGCCAAGCAGCTCTGCAGCAGACCAAGG
AGATGGACCTCAGCGTGGTGCGGCTCATGTTTACAGCTTTTCTTCCGGATAGCAC
TGGCAGCTTCACAAGGCGCCTGGAACCCGTGGTATCAGACGCCATCTATGACAGT
AAAGCCCCCAATGCATCCAACTTGAAAATTGTAAGAATGGACAGGACAGCTGGA
TGTGTGACTGGAGGGGAGGAAATTTATCTTCTTTGTGACAAAGTTCAGAAAGATG
ACATCCAGATTCGATTTTATGAAGAGGAAGAAAATGGTGGAGTCTGGGAAGGAT
TTGGAGATTTTTCCCCCACAGATGTTCATAGACAATTTGCCATTGTCTTCAAAACT
CCAAAGTATAAAGATATTAATATTACAAAACCAGCCTCTGTGTTTGTCCAGCTTC
GGAGGAAATCTGACTTGGAAACTAGTGAACCAAAACCTTTCCTCTACTATCCTGA
AATCAAAGATAAAGAAGAAGTGCAGAGGAAACGTCAGAAGCTCATGCCCAATTT
TTCGGATAGTTTCGGCGGTGGTAGTGGTGCCGGAGCTGGAGGCGGAGGCATGTTT
GGTAGTGGCGGTGGAGGAGGGGGCACTGGAAGTACAGGTCCAGGGTATAGCTTC
CCACACTATGGATTTCCTACTTATGGTGGGATTACTTTCCATCCTGGAACTACTAA
ATCTAATGCTGGGATGAAGCATGGAACCATGGACACTGAATCTAAAAAGGACCC
TGAAGGTTGTGACAAAAGTGATGACAAAAACACTGTAAACCTCTTTGGGAAAGT
TATTGAAACCACAGAGCAAGATCAGGAGCCCAGCGAGGCCACCGTTGGGAATGG
TGAGGTCACTCTAACGTATGCAACAGGAACAAAAGAAGAGAGTGCTGGAGTTCA
GGATAACCTCTTTCTAGAGAAGGCTATGCAGCTTGCAAAGAGGCATGCCAATGC
CCTTTTCGACTACGCGGTGACAGGAGACGTGAAGATGCTGCTGGCCGTCCAGCGC
CATCTCACTGCTGTGCAGGATGAGAATGGGGACAGTGTCTTACACTTAGCAATCA
TCCACCTTCATTCTCAACTTGTGAGGGATCTACTAGAAGTCACATCTGGTTTGATT
TCTGATGACATTATCAACATGAGAAATGATCTGTACCAGACGCCCTTGCACTTGG
CAGTGATCACTAAGCAGGAAGATGTGGTGGAGGATTTGCTGAGGGCTGGGGCCG
ACCTGAGCCTTCTGGACCGCTTGGGTAACTCTGTTTTGCACCTAGCTGCCAAAGA
AGGACATGATAAAGTTCTCAGTATCTTACTCAAGCACAAAAAGGCAGCACTACTT
CTTGACCACCCCAACGGGGACGGTCTGAATGCCATTCATCTAGCCATGATGAGCA
ATAGCCTGCCATGTTTGCTGCTGCTGGTGGCCGCTGGGGCTGACGTCAATGCTCA
GGAGCAGAAGTCCGGGCGCACAGCACTGCACCTGGCTGTGGAGCACGACAACAT
CTCATTGGCAGGCTGCCTGCTCCTGGAGGGTGATGCCCATGTGGACAGTACTACC
TACGATGGAACCACACCCCTGCATATAGCAGCTGGGAGAGGGTCCACCAGGCTG
GCAGCTCTTCTCAAAGCAGCAGGAGCAGATCCCCTGGTGGAGAACTTTGAGCCTC
TCTATGACCTGGATGACTCTTGGGAAAATGCAGGAGAGGATGAAGGAGTTGTGC
CTGGAACCACGCCTCTAGATATGGCCACCAGCTGGCAGGTATTTGACATATTAAA
TGGGAAACCATATGAGCCAGAGTTTACATCTGATGATTTACTAGCACAAGGAGA
CATGAAACAGCTGGCTGAAGATGTGAAGCTGCAGCTGTATAAGTTACTAGAAAT
TCCTGATCCAGACAAAAACTGGGCTACTCTGGCGCAGAAATTAGGTCTGGGGAT
ACTTAATAATGCCTTCCGGCTGAGTCCTGCTCCTTCCAAAACACTTATGGACAAC
TATGAGGTCTCTGGGGGTACAGTCAGAGAGCTGGTGGAGGCCCTGAGACAAATG
GGCTACACCGAAGCAATTGAAGTGATCCAGGCAGCCTCCAGCCCAGTGAAGACC
ACCTCTCAGGCCCACTCGCTGCCTCTCTCGCCTGCCTCCACAAGGCAGCAAATAG
ACGAGCTCCGAGACAGTGACAGTGTCTGCGACAGCGGCGTGGAGACATCCTTCC
GCAAACTCAGCTTTACCGAGTCTCTGACCAGTGGTGCCTCACTGCTAACTCTCAA
CAAAATGCCCCATGATTATGGGCAGGAAGGACCTCTAGAAGGCAAAATTTAGCC
TGCTGACAATTTCCCACACCGTGTAAACCAAAGCCCTAAAATTCCACTGCGTTGT
CCACAAGACAGAAGCTGAAGTGCATCCAAAGGTGCTCAGAGAGCCGGCCCGCCT
GAATCATTCTCGATTTAACTCGAGACCTTTTCAACTTGGCTTCCTTTCTTGGTTCA
TAAATGAATTTTAGTTTGGTTCACTTACAGATAGTATCTAGCAATCACAACACTG
GCTGAGCGGATGCATCTGGGGATGAGGTTGCTTACTAAGCTTTGCCAGCTGCTGC
TGGATCACAGCTGCTTTCTGTTGTCATTGCTGTTGTCCCTCTGCTACGTTCCTATT
GTCATTAAAGGTATCACGGTCGCCACCTGGCATTCCTTCTGACCACAGCATCATT
TTGCATTCAAATTAAGGGTTAAGAAAAGAGATATTTTAAAATGAGAGTCACTTGA
TGTGCCATTTTAAAAAAAAAGGCATATTGCTTTTTCTAATGTGGTTATTTCTCTGA
TTTGCAAAAAAAAAAAAAAAAAAAAAATACTTGTCAATATTTAAACATGGTTAC
AATCATTGCTGAAAATGGTATTTTCCCCCTTTTCTGCATTTTGCTATTGTAAATAT
GTTTTTTAGATCAAATACTTTAAAGGAAAAAATGTTGGATTTATAAATGCTATTTTTTATTT
TACTTTTATAATAAAAGGAAAAGCAAATTGATGACCTCAAAAAAAAAAAAAAAAAA.

H. sapiens nuclear factor of kappa light polypeptide gene enhancer in B-cells 2 (p49/p100) (NF-κB2), mRNA, (GI 19923222):

(SEQ ID NO.: 11)
ACTTTCCTGCCCCTTCCCCGGCCAAGCCCAACTCCGGATCTCGCTCTCCACCGGAT
CTCACCCGCCACACCCGGACAGGCGGCTGGAGGAGGCGGGCGTCTAAAATTCTG
GGAAGCAGAACCTGGCCGGAGCCACTAGACAGAGCCGGGCCTAGCCCAGAGAC
ATGGAGAGTTGCTACAACCCAGGTCTGGATGGTATTATTGAATATGATGATTTCA
AATTGAACTCCTCCATTGTGGAACCCAAGGAGCCAGCCCCAGAAACAGCTGATG
GCCCCTACCTGGTGATCGTGGAACAGCCTAAGCAGAGAGGCTTCCGATTTCGATA
TGGCTGTGAAGGCCCCTCCCATGGAGGACTGCCCGGTGCCTCCAGTGAGAAGGG
CCGAAAGACCTATCCCACTGTCAAGATCTGTAACTACGAGGGACCAGCCAAGAT
CGAGGTGGACCTGGTAACACACAGTGACCCACCTCGTGCTCATGCCCACAGTCTG
GTGGGCAAGCAATGCTCGGAGCTGGGGATCTGCGCCGTTTCTGTGGGGCCCAAG
GACATGACTGCCCAATTTAACAACCTGGGTGTCCTGCATGTGACTAAGAAGAAC
ATGATGGGGACTATGATACAAAAACTTCAGAGGCAGCGGCTCCGCTCTAGGCCC
CAGGGCCTTACGGAGGCCGAGCAGCGGGAGCTGGAGCAAGAGGCCAAAGAACT
GAAGAAGGTGATGGATCTGAGTATAGTGCGGCTGCGCTTCTCTGCCTTCCTTAGA
GCCAGTGATGGCTCCTTCTCCCTGCCCCTGAAGCCAGTCACCTCCCAGCCCATCC
ATGATAGCAAATCTCCGGGGGCATCAAACCTGAAGATTTCTCGAATGGACAAGA
CAGCAGGCTCTGTGCGGGGTGGAGATGAAGTTTATCTGCTTTGTGACAAGGTGCA
GAAAGATGACATTGAGGTTCGGTTCTATGAGGATGATGAGAATGGATGGCAGGC
CTTTGGGGACTTCTCTCCCACAGATGTGCATAAACAGTATGCCATTGTGTTCCGG
ACACCCCCCTATCACAAGATGAAGATTGAGCGGCCTGTAACAGTGTTTCTGCAAC
TGAAACGCAAGCGAGGAGGGGACGTGTCTGATTCCAAACAGTTCACCTATTACC
CTCTGGTGGAAGACAAGGAAGAGGTGCAGCGGAAGCGGAGGAAGGCCTTGCCC
ACCTTCTCCCAGCCCTTCGGGGGTGGCTCCCACATGGGTGGAGGCTCTGGGGGTG
CAGCCGGGGGCTACGGAGGAGCTGGAGGAGGTGGCAGCCTCGGTTTCTTCCCCT
CCTCCCTGGCCTACAGCCCCTACCAGTCCGGCGCGGGCCCCATGCGGTGCTACCC
GGGAGGCGGGGGCGGGGCGCAGATGGCCGCCACGGTGCCCAGCAGGGACTCCG
GGGAGGAAGCCGCGGAGCCGAGCGCCCCCTCCAGGACCCCCCAGTGCGAGCCGC
AGGCCCCGGAGATGCTGCAGCGAGCTCGAGAGTACAACGCGCGCCTGTTCGGCC
TGGCGCACGCAGCCCCGAGCCCTACTCGACTACTGCGTCACCGCGGACGCCGCG
CGCTGCTGGCGGGACAGCGCCACCTGCTGACGGCGCAGGACGAGAACGGAGACA
CACCACTGCACCTAGCCATCATCCACGGGCAGACCAGTGTCATTGAGCAGATAGT
CTATGTCATCCACCACGCCCAGGACCTCGGCGTTGTCAACCTCACCAACCACCTG
CACCAGACGCCCCTGCACCTGGCGGTGATCACGGGGCAGACGAGTGTGGTGAGC
TTTCTGCTGCGGGTAGGTGCAGACCCAGCTCTGCTGGATCGGCATGGAGACTCAG
CCATGCATCTGGCGCTGCGGGCAGGCGCTGGTGCTCCTGAGCTGCTGCGTGCACT
GCTTCAGAGTGGAGCTCCTGCTGTGCCCCAGCTGTTGCATATGCCTGACTTTGAG
GGACTGTATCCAGTACACCTGGCGGTCCGAGCCCGAAGCCCTGAGTGCCTGGATC
TGCTGGTGGACAGTGGGGCTGAAGTGGAGGCCACAGAGCGGCAGGGGGGACGA
ACAGCCTTGCATCTAGCCACAGAGATGGAGGAGCTGGGGTTGGTCACCCATCTG
GTCACCAAGCTCCGGGCCAACGTGAACGCTCGCACCTTTGCGGGAAACACACCC
CTGCACCTGGCAGCTGGACTGGGGTACCCGACCCTCACCCGCCTCCTTCTGAAGG
CTGGTGCTGACATCCATGCTGAAAACGAGGAGCCCCTGTGCCCACTGCCTTCACC
CCCTACCTCTGATAGCGACTCGGACTCTGAAGGGCCTGAGAAGGACACCCGAAG
CAGCTTCCGGGGCCACACGCCTCTTGACCTCACTTGCAGCACCTTGGTGAAGACC
TTGCTGCTAAATGCTGCTCAGAACACCATGGAGCCACCCCTGACCCCGCCCAGCC
CAGCAGGGCCGGGACTGTCACTTGGTGATACAGCTCTGCAGAACCTGGAGCAGC
TGCTAGACGGGCCAGAAGCCCAGGGCAGCTGGGCAGAGCTGGCAGAGCGTCTGG
GGCTGCGCAGCCTGGTAGACACGTACCGACAGACAACCTCACCCAGTGGCAGCC
TCCTGCGCAGCTACGAGCTGGCTGGCGGGGACCTGGCAGGTCTACTGGAGGCCC
TGTCTGACATGGGCCTAGAGGAGGGAGTGAGGCTGCTGAGGGGTCCAGAAACCC
GAGACAAGCTGCCCAGCACAGAGGTGAAGGAAGACAGTGCGTACGGGAGCCAG
TCAGTGGAGCAGGAGGCAGAGAAGCTGGGCCCACCCCCTGAGCCACCAGGAGG
GCTCTCGCACGGGCACCCCCAGCCTCAGGTGACTGACCTGCTGCCTGCCCCCAGCCCC
CTTCCCGGACCCCCTGTACAGCGTCCCCACCTATTTCAAATCTTATTTAACACCCCACAC
CCACCCCTCAGTTGGGACAAATAAAGGATTCTCATGGGAAGGGGAGGACCCCGAATTCCT.

H. sapiens nuclear factor of kappa light polypeptide gene enhancer in B-cells 2 (p49/p 100) (NF-κB2), transcript variant 2, mRNA, (GI 117320526):

(SEQ ID NO.: 12)
GGTATTTTCGGGACTTTCCTAAGCTGCTCTAACTTTCCTGCCCCTTCCCCGGCCAA
GCCCAACTCCGGATCTCGCTCTCCACCGGATCTCACCCGCCACACCCGGACAGGC
GGCTGGAGGAGGCGGGCGTCTAAAATTCTGGGAAGCAGAACCTGGCCGGAGCCA
CTAGACAGAGCCGGGCCTAGCCCAGAGACATGGAGAGTTGCTACAACCCAGGTC
TGGATGGTATTATTGAATATGATGATTTCAAATTGAACTCCTCCATTGTGGAACC
CAAGGAGCCAGCCCCAGAAACAGCTGATGGCCCCTACCTGGTGATCGTGGAACA
GCCTAAGCAGAGAGGCTTCCGATTTCGATATGGCTGTGAAGGCCCCTCCCATGGA
GGACTGCCCGGTGCCTCCAGTGAGAAGGGCCGAAAGACCTATCCCACTGTCAAG
ATCTGTAACTACGAGGGACCAGCCAAGATCGAGGTGGACCTGGTAACACACAGT
GACCCACCTCGTGCTCATGCCCACAGTCTGGTGGGCAAGCAATGCTCGGAGCTGG
GGATCTGCGCCGTTTCTGTGGGGCCCAAGGACATGACTGCCCAATTTAACAACCT
GGGTGTCCTGCATGTGACTAAGAAGAACATGATGGGGACTATGATACAAAAACT
TCAGAGGCAGCGGCTCCGCTCTAGGCCCCAGGGCCTTACGGAGGCCGAGCAGCG
GGAGCTGGAGCAAGAGGCCAAAGAACTGAAGAAGGTGATGGATCTGAGTATAG
TGCGGCTGCGCTTCTCTGCCTTCCTTAGAGCCAGTGATGGCTCCTTCTCCCTGCCC
CTGAAGCCAGTCATCTCCCAGCCCATCCATGACAGCAAATCTCCGGGGGCATCAA
ACCTGAAGATTTCTCGAATGGACAAGACAGCAGGCTCTGTGCGGGGTGGAGATG
AAGTTTATCTGCTTTGTGACAAGGTGCAGAAAGATGACATTGAGGTTCGGTTCTA
TGAGGATGATGAGAATGGATGGCAGGCCTTTGGGGACTTCTCTCCCACAGATGTG
CATAAACAGTATGCCATTGTGTTCCGGACACCCCCCTATCACAAGATGAAGATTG
AGCGGCCTGTAACAGTGTTTCTGCAACTGAAACGCAAGCGAGGAGGGGACGTGT
CTGATTCCAAACAGTTCACCTATTACCCTCTGGTGGAAGACAAGGAAGAGGTGC
AGCGGAAGCGGAGGAAGGCCTTGCCCACCTTCTCCCAGCCCTTCGGGGGTGGCT
CCCACATGGGTGGAGGCTCTGGGGGTGCAGCCGGGGGCTACGGAGGAGCTGGAG
GAGGTGGCAGCCTCGGTTTCTTCCCCTCCTCCCTGGCCTACAGCCCCTACCAGTCC
GGCGCGGGCCCCATGGGCTGCTACCCGGGAGGCGGGGGCGGGGCGCAGATGGCC
GCCACGGTGCCCAGCAGGGACTCCGGGGAGGAAGCCGCGGAGCCGAGCGCCCCC
TCCAGGACCCCCCAGTGCGAGCCGCAGGCCCCGGAGATGCTGCAGCGAGCTCGA
GAGTACAACGCGCGCCTGTTCGGCCTGGCGCAGCGCAGCGCCCGAGCCCTACTC
GACTACGGCGTCACCGCGGACGCGCGCGCGCTGCTGGCGGGACAGCGCCACCTG
CTGACGGCGCAGGACGAGAACGGAGACACACCACTGCACCTAGCCATCATCCAC
GGGCAGACCAGTGTCATTGAGCAGATAGTCTATGTCATCCACCACGCCCAGGAC
CTCGGCGTTGTCAACCTCACCAACCACCTGCACCAGACGCCCCTGCACCTGGCGG
TGATCACGGGGCAGACGAGTGTGGTGAGCTTTCTGCTGCGGGTAGGTGCAGACC
CAGCTCTGCTGGATCGGCATGGAGACTCAGCCATGCATCTGGCGCTGCGGGCAG
GCGCTGGTGCTCCTGAGCTGCTGCGTGCACTGCTTCAGAGTGGAGCTCCTGCTGT
GCCCCAGCTGTTGCATATGCCTGACTTTGAGGGACTGTATCCAGTACACCTGGCG
GTCCGAGCCCGAAGCCCTGAGTGCCTGGATCTGCTGGTGGACAGTGGGGCTGAA
GTGGAGGCCACAGAGCGGCAGGGGGGACGAACAGCCTTGCATCTAGCCACAGA
GATGGAGGAGCTGGGGTTGGTCACCCATCTGGTCACCAAGCTCCGGGCCAACGT
GAACGCTCGCACCTTTGCGGGAAACACACCCCTGCACCTGGCAGCTGGACTGGG
GTACCCGACCCTCACCCGCCTCCTTCTGAAGGCTGGTGCTGACATCCATGCTGAA
AACGAGGAGCCCCTGTGCCCACTGCCTTCACCCCCTACCTCTGATAGCGACTCGG
ACTCTGAAGGGCCTGAGAAGGACACCCGAAGCAGCTTCCGGGGCCACACGCCTC
TTGACCTCACTTGCAGCACCAAGGTGAAGACCTTGCTGCTAAATGCTGCTCAGAA
CACCATGGAGCCACCCCTGACCCCGCCCAGCCCAGCAGGGCCGGGACTGTCACT
TGGTGATACAGCTCTGCAGAACCTGGAGCAGCTGCTAGACGGGCCAGAAGCCCA
GGGCAGCTGGGCAGAGCTGGCAGAGCGTCTGGGGCTGCGCAGCCTGGTAGACAC
GTACCGACAGACAACCTCACCCAGTGGCAGCCTCCTGCGCAGCTACGAGCTGGC
TGGCGGGGACCTGGCAGGTCTACTGGAGGCCCTGTCTGACATGGGCCTAGAGGA
GGGAGTGAGGCTGCTGAGGGGTCCAGAAACCCGAGACAAGCTGCCCAGCACAG
AGGTGAAGGAAGACAGTGCGTACGGGAGCCAGTCAGTGGAGCAGGAGGCAGAG
AAGCTGGGCCCACCCCCTGAGCCACCAGGAGGGCTCTGCCACGGGCACCCCCAG
CCTCAGGTGCACTGACCTGCTGCCTGCCCCCAGCCCCCTTCCCGGACCCCCTGTA
CAGCGTCCCCACCTATTTCAAATCTTATTTAACACCCCACACCCACCCCTCAGTTGGGACA
AATAAAGGATTCTCATGGGAAGGGGAGGACCCCTCCTTCCCAACTTAAAAAAAAAAAAA.

H. sapiens v-rel reticuloendotheliosis viral oncogene homolog A, nuclear factor of kappa light polypeptide gene enhancer in B-cells 3, p65 (avian) (RELA), mRNA, (GI 46430498):

(SEQ ID NO.: 13)
GGCGAATGGCTCGTCTGTAGTGCACGCCGCGGGCCCAGCTGCGACCCCGGCCCC
GCCCCCGGGACCCCGGCCATGGACGAACTGTTCCCCCTCATCTTCCCGGCAGAGC
CAGCCCAGGCCTCTGGCCCCTATGTGGAGATCATTGAGCAGCCCAAGCAGCGGG
GCATGCGCTTCCGCTACAAGTGCGAGGGGCGCTCCGCGGGCAGCATCCCAGGCG
AGAGGAGCACAGATACCACCAAGACCCACCCCACCATCAAGATCAATGGCTACA
CAGGACCAGGGACAGTGCGCATCTCCCTGGTCACCAAGGACCCTCCTCACCGGC
CTCACCCCCACGAGCTTGTAGGAAAGGACTGCCGGGATGGCTTCTATGAGGCTG
AGCTCTGCCCGGACCGCTGCATCCACAGTTTCCAGAACCTGGGAATCCAGTGTGT
GAAGAAGCGGGACCTGGAGCAGGCTATCAGTCAGCGCATCCAGACCAACAACAA
CCCCTTCCAAGTTCCTATAGAAGAGCAGCGTGGGGACTACGACCTGAATGCTGTG
CGGCTCTGCTTCCAGGTGACAGTGCGGGACCCATCAGGCAGGCCCCTCCGCCTGC
CGCCTGTCCTTCCTCATCCCATCTTTGACAATCGTGCCCCCAACACTGCCGAGCTC
AAGATCTGCCGAGTGAACCGAAACTCTGGCAGCTGCCTCGGTGGGGATGAGATC
TTCCTACTGTGTGACAAGGTGCAGAAAGAGGACATTGAGGTGTATTTCACGGGA
CCAGGCTGGGAGGCCCGAGGCTCCTTTTCGCAAGCTGATGTGCACCGACAAGTG
GCCATTGTGTTCCGGACCCCTCCCTACGCAGACCCCAGCCTGCAGGCTCCTGTGC
GTGTCTCCATGCAGCTGCGGCGGCCTTCCGACCGGGAGCTCAGTGAGCCCATGGA
ATTCCAGTACCTGCCAGATACAGACGATCGTCACCGGATTGAGGAGAAACGTAA
AAGGACATATGAGACCTTCAAGAGCATCATGAAGAAGAGTCCTTTCAGCGGACC
CACCGACCCCCGGCCTCCACCTCGACGCATTGCTGTGCCTTCCCGCAGCTCAGCT
TCTGTCCCCAAGCCAGCACCCCAGCCCTATCCCTTTACGTCATCCCTGAGCACCA
TCAACTATGATGAGTTTCCCACCATGGTGTTTCCTTCTGGGCAGATCAGCCAGGC
CTCGGCCTTGGCCCCGGCCCCTCCCCAAGTCCTGCCCCAGGCTCCAGCCCCTGCC
CCTGCTCCAGCCATGGTATCAGCTCTGGCCCAGGCCCCAGCCCCTGTCCCAGTCC
TAGCCCCAGGCCCTCCTCAGGCTGTGGCCCCACCTGCCCCCAAGCCCACCCAGGC
TGGGGAAGGAACGCTGTCAGAGGCCCTGCTGCAGCTGCAGTTTGATGATGAAGA
CCTGGGGGCCTTGCTTGGCAACAGCACAGACCCAGCTGTGTTCACAGACCTGGCA
TCCGTCGACAACTCCGAGTTTCAGCAGCTGCTGAACCAGGGCATACCTGTGGCCC
CCCACACAACTGAGCCCATGCTGATGGAGTACCCTGAGGCTATAACTCGCCTAGT
GACAGGGGCCCAGAGGCCCCCCGACCCAGCTCCTGCTCCACTGGGGGCCCCGGG
GCTCCCCAATGGCCTCCTTTCAGGAGATGAAGACTTCTCCTCCATTGCGGACATG
GACTTCTCAGCCCTGCTGAGTCAGATCAGCTCCTAAGGGGGTGACGCCTGCCCTCCCCAG
AGCACTGG.

H. sapiens v-rel reticuloendotheliosis viral oncogene homolog B, nuclear factor of kappa light polypeptide gene enhancer in B-cells 3 (avian) (RELB), mRNA, (GI 35493877):

(SEQ ID NO.: 14)
CGCGCCCCGCGCAGCCCCGGGCGCCGCGCGTCCTGCCCGGCCTGCGGCCCCAGC
CCTTGCGCCGCTCGTCCGACCCGCGATCGTCCACCAGACCGTGCCTCCCGGCCGC
CCGGCCGGCCCGCGTGCATGCTTCGGTCTGGGCCAGCCTCTGGGCCGTCCGTCCC
CACTGGCCGGGCCATGCCGAGTCGCCGCGTCGCCAGACCGCCGGCTGCGCCGGA
GCTGGGGGCCTTAGGGTCCCCCGACCTCTCCTCACTCTCGCTCGCCGTTTCCAGG
AGCACAGATGAATTGGAGATCATCGACGAGTACATCAAGGAGAACGGCTTCGGC
CTGGACGGGGGACAGCCGGGCCCGGGCGAGGGGCTGCCACGCCTGGTGTCTCGC
GGGGCTGCGTCCCTGAGCACGGTCACCCTGGGCCCTGTGGCGCCCCCAGCCACGC
CGCCGCCTTGGGGCTGCCCCCTGGGCCGACTAGTGTCCCCAGCGCCGGGCCCGGG
CCCGCAGCCGCACCTGGTCATCACGGAGCAGCCCAAGCAGCGCGGCATGCGCTT
CCGCTACGAGTGCGAGGGCCGCTCGGCCGGCAGCATCCTTGGGGAGAGCAGCAC
CGAGGCCAGCAAGACGCTGCCCGCCATCGAGCTCCGGGATTGTGGAGGGCTGCG
GGAGGTGGAGGTGACTGCCTGCCTGGTGTGGAAGGACTGGCCTCACCGAGTCCA
CCCCCACAGCCTCGTGGGGAAAGACTGCACCGACGGCATCTGCAGGGTGCGGCT
CCGGCCTCACGTCAGCCCCCGGCACAGTTTTAACAACCTGGGCATCCAGTGTGTG
AGGAAGAAGGAGATTGAGGCTGCCATTGAGCGGAAGATTCAACTGGGCATTGAC
CCCTACAACGCTGGGTCCCTGAAGAACCATCAGGAAGTAGACATGAATGTGGTG
AGGATCTGCTTCCAGGCCTCATATCGGGACCAGCAGGGACAGATGCGCCGGATG
GATCCTGTGCTTTCCGAGCCCGTCTATGACAAGAAATCCACAAACACATCAGAGC
TGCGGATTTGCCGAATTAACAAGGAAAGCGGGCCGTGCACCGGTGGCGAGGAGC
TCTACTTGCTCTGCGACAAGGTGCAGAAAGAGGACATATCAGTGGTGTTCAGCA
GGGCCTCCTGGGAAGGTCGGGCTGACTTCTCCCAGGCCGACGTGCACCGCCAGA
TTGCCATTGTGTTCAAGACGCCGCCCTACGAGGACCTGGAGATTGTCGAGCCCGT
GACAGTCAACGTCTTCCTGCAGCGGCTCACCGATGGGGTCTGCAGCGAGCCATTG
CCTTTCACGTACCTGCCTCGCGACCATGACAGCTACGGCGTGGACAAGAAGCGG
AAACGGGGGATGCCCGACGTCCTTGGGGAGCTGAACAGCTCTGACCCCCATGGC
ATCGAGAGCAAACGGCGGAAGAAAAAGCCGGCCATCCTGGACCACTTCCTGCCC
AACCACGGCTCAGGCCCGTTCCTCCCGCCGTCAGCCCTGCTGCCAGACCCTGACT
TCTTCTCTGGCACCGTGTCCCTGCCCGGCCTGGAGCCCCCTGGCGGGCCTGACCT
CCTGGACGATGGCTTTGCCTACGACCCTACGGCCCCCACACTCTTCACCATGCTG
GACCTGCTGCCCCCGGCACCGCCACACGCTAGCGCTGTTGTGTGCAGCGGAGGTG
CCGGGGCCGTGGTTGGGGAGACCCCCGGCCCTGAACCACTGACACTGGACTCGT
ACCAGGCCCCGGGCCCCGGGGATGGAGGCACCGCCAGCCTTGTGGGCAGCAACA
TGTTCCCCAATCATTACCGCGAGGCGGCCTTTGGGGGCGGCCTCCTATCCCCGGG
GCCTGAAGCCACGTAGCCCCGCGATGCCAGAGGAGGGGCACTGGGTGGGGAGG
GAGGTGGAGGAGCCGTGCAATCCCAACCAGGATGTCTAGCACCCCCATCCCCTT
GGCCCTTCCTCATGCTTCTGAAGTGGACATATTCAGCCTTGGCGAGAAGCTCCGT
TGCACGGGTTTCCCCTTGAGCCCATTTTACAGATGAGGAAACTGAGTCCGGAGAG
GAAAAGGGACATGGCTCCCGTGCACTAGCTTGTTACAGCTGCCTCTGTCCCCACA
TGTGGGGGCACCTTCTCCAGTAGGATTCGGAAAAGATTGTACATATGGGAGGAG
GGGGCAGATTCCTGGCCCTCCCTCCCCAGACTTGAAGGTGGGGGGTAGGTTGGTTGTTCA
GAGTCTTCCCAATAAAGATGAGTTTTTGAGCCTCAAAAAAAAAAAAAAAAA.

In some embodiments, a ribozyme may be a CXCL12 and/or CXCR4 antagonist. A ribozyme is designed to catalytically cleave target mRNA transcripts may be used to prevent translation of a target mRNA and/or expression of a target (see, e.g., PCT Publication WO 90/11364; and Sarver et al., 1990, Science 247:1222; both of which arc incorporated herein by reference). Any of the RNAi-inducing entity targets described herein may be utilized as a ribozyme target in accordance with the present invention.

In some embodiments, endogenous target gene expression may be reduced by targeting deoxyribonucleotide sequences complementary to the regulatory region of a target gene (i.e., a target gene's promoter and/or enhancers) to form triple helical structures that prevent transcription of a target gene (see generally, Helene, 1991, Anticancer Drug Des. 6:569; Helene et al., 1992, Ann, N.Y. Acad. Sci. 660:27; and Maher, 1992, Bioassays 14:807; all of which are incorporated herein by reference). Any of the RNAi-inducing entity targets described herein may be utilized as a target for formation of triple helical structures in accordance with the present invention.

Nucleic acid CXCL12 and/or CXCR4 antagonists in accordance with the present invention (including RNAi-inducing agents, ribozymes, triple-helix inducing agents, etc., described in further detail below) may be prepared according to any available technique including, but not limited to chemical synthesis, enzymatic synthesis, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005).

A nucleic acid that forms a nucleic acid CXCL12 and/or CXCR4 antagonist may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of a nucleic acid CXCL12 and/or CXCR4 antagonist can be replaced with a hydrocarbon linker or a polyether linker provided that the binding affinity, selectivity, and/or other functional characteristics of the nucleic acid CXCL12 and/or CXCR4 antagonist is not substantially reduced by the substitution.

It will be appreciated by those of ordinary skill in the art that nucleic acids in accordance with the present invention may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used. See Crooke, S. (ed.) Antisense Drug Technology: Principles, Strategies, and Applications (1^{st} ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR, R, halo, SH, SR₁, NH₂, NH_{R}, NR₂ or CN, wherein R is C₁-C₆ alkyl, alkenyl, or alkynyl, and halo is F, Cl, Br or I. Examples of modified linkages include phosphorothioate and 5′-N-phosphoramidite linkages.

Nucleic acids comprising a variety of different nucleotide analogs, modified backbones, or non-naturally occurring internucleoside linkages can be utilized in accordance with the present invention. Nucleic acids in accordance with the present invention may include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein arc utilized to reduce and/or prevent digestion by nucleases (e.g., exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in a nucleic acid. One of ordinary skill in the art will appreciate that the nucleotide analogs or other modification(s) may be located at any position(s) of a nucleic acid such that the function of the nucleic acid is not substantially affected. The modified region may be at the 5′-end and/or the 3′-end of one or both strands. For example, modified nucleic acids in which approximately 1 to approximately 5 residues at the 5′ and/or 3′ end of either of both strands are nucleotide analogs and/or have a backbone modification have been employed. A modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the present invention may, for example, comprise a modification to a sugar, nucleoside, or internucleoside linkage such as those described in U.S. Patent Publications 2003/0175950, 2004/0192626, 2004/0092470, 2005/0020525, and 2005/0032733 (all of which arc incorporated herein by reference). The present invention encompasses the use of any nucleic acid having any one or more of the modifications described therein. For example, a number of terminal conjugates, e.g., lipids such as cholesterol, lithocholic acid, aluric acid, or long alkyl branched chains have been reported to improve cellular uptake. Analogs and modifications may be tested, e.g., using any appropriate assay known in the art. In some embodiments, nucleic acids in accordance with the present invention may comprise one or more non-natural nucleoside linkages. In some embodiments, one or more internal nucleotides at the 3′-end, 5′-end, or both 3′- and 5′-ends of a nucleic acid are inverted to yield linkages such as a 3′-3′ linkage or a 5′-5′ linkage.

In some embodiments, nucleic acids in accordance with the present invention are not synthetic, but are naturally-occurring entities that have been isolated from their natural environments.

Carbohydrate CXCL12 and/or CXCR4 Antagonists

In some embodiments, a CXCL12 and/or CXCR4 antagonist in accordance with the present invention may comprise a carbohydrate. Any carbohydrate that negatively affects the ability of CXCL12 to bind to CXCR4 is a CXCL12 and/or CXCR4 antagonist in accordance with the present invention. Any carbohydrate that negatively affects the activity of CXCR12 and/or CXCR4 is a CXCL12 and/or CXCR4 antagonist in accordance with the present invention. In certain embodiments, a carbohydrate that, upon administration to a subject, causes mobilization of naïve T cells and Tregs from bone marrow to peripheral lymphoid organs is a CXCL12 and/or CXCR4 antagonist in accordance with the present invention.

In some embodiments, a carbohydrate may be a polysaccharide comprising simple sugars (or their derivatives) connected by glycosidic bonds, as known in the art. Such sugars may include, but are not limited to, glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan.

In some embodiments, a carbohydrate may be aminated, carboxylated, and/or sulfated. In some embodiments, hydrophilic polysaccharides can be modified to become hydrophobic by introducing a large number of side-chain hydrophobic groups. In some embodiments, a hydrophobic carbohydrate may include cellulose acetate, pullulan acetate, konjac acetate, amylose acetate, and dextran acetate.

Lipid CXCL12 and/or CXCR4 Antagonists

In some embodiments, a CXCL12 and/or CXCR4 antagonist in accordance with the present invention may comprise one or more fatty acid groups or salts thereof. Any lipid that negatively affects the ability of CXCL12 to bind to CXCR4 is a CXCL12 and/or CXCR4 antagonist in accordance with the present invention. Any lipid that negatively affects the activity of CXCR12 and/or CXCR4 is a CXCL12 and/or CXCR4 antagonist in accordance with the present invention. In certain embodiments, a lipid that, upon administration to a subject, causes mobilization of naïve T cells and Tregs from bone marrow to peripheral lymphoid organs is a CXCL12 and/or CXCR4 antagonist in accordance with the present invention.

In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid group may be a C₁₅-C25 fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

Identification of Novel CXCL12 or CXCR4 Antagonists

The present invention provides methods of identifying novel CXCL12 and/or CXCR4 antagonists. In some embodiments, methods in accordance with the invention involve screening for novel CXCL12 and/or CXCR4 antagonists by identifying substances that improve and/or treat symptoms of autoimmune disorders (e.g., diabetes). In some embodiments, methods in accordance with the invention involve screening for novel CXCL12 and/or CXCR4 antagonists by identifying substances that alter trafficking of T cells and/or hematopoietic stem cells. In some embodiments, methods in accordance with the invention involve identifying substances that affect the ability of CXCL12 and/or CXCR4 to interact with their natural binding partners. In some embodiments, methods in accordance with the invention involve identifying substances that modulate CXCL12 and/or CXCR4 expression and/or levels.

As used herein, the phrase “test substance” refers to (1) a CXCL12 and/or CXCR4 protein, a nucleic acid encoding CXCL12 and/or CXCR4, and/or homolog, portion, variant, mutant, and/or derivative thereof; and/or (2) a natural binding partner of CXCL12 and/or CXCR4, a nucleic acid encoding a natural binding partner of CXCL12 and/or CXCR4, and/or a homolog, portion, variant, mutant, and/or derivative thereof. In some embodiments, a test substance is a protein or peptide comprising a CXCL12-binding portion of CXCR4. In some embodiments, a test substance is a protein or peptide comprising a CXCR4-binding portion of CXCL12.

The efficacy of a test substance may be assessed by generating dose response curves from data obtained using various concentrations of the test substance. Moreover, a control assay may be performed to provide a baseline for comparison. In a control assay, the assay may be performed in the absence of a test substance.

In some embodiments, CXCL12 and/or CXCR4 antagonists in accordance with the invention inhibit and/or activate CXCL12 and/or CXCR4 activity by at least about 10%, about 20%, about 30%, about 30%, about 40%, about 60%, about 70%, about 75%, about 80%, about 85% about 90%, about 95%, about 98%, about 99%, or more as compared with the activity observed under otherwise identical conditions lacking a test substance.

It will, of course, be understood that all screening methods in accordance with the present invention are useful in themselves notwithstanding the fact that effective candidates may not be found. The invention provides methods for screening for such candidates, not solely methods of finding them.

Test substances may be isolated from natural sources, such as animals, bacteria, fungi, plants, and/or marine samples may be assayed for the presence of potentially useful CXCL12 and/or CXCR4 antagonists. It will be understood that test substances to be screened could be derived and/or synthesized from chemical compositions or man-made substances.

Rational Drug Design

As used herein the term “candidate substance” refers to any substance that may potentially inhibit an autoimmune disorder (e.g., diabetes) and/or act as a CXCL12 and/or CXCR4 antagonist. A candidate substance may be a protein, an antibody, a nucleic acid, a small molecule, carbohydrate, lipid, virus, and/or characteristic portion thereof. It may prove to be the case that the most useful candidate substances will be substances that arc structurally related to CXCL12, CXCR4, their binding partners, their upstream effectors, and/or their downstream effectors, i.e., mimics. Using lead compounds to help develop improved compounds is known as “rational drug design” and includes not only comparisons with known antagonists, but predictions relating to the structure of target substances.

In some embodiments, rational drug design may be used to predict and/or produce structural analogs of known biologically-active candidate substances. By creating such analogs, it is possible to fashion drugs which may be more active and/or stable than natural substances, may have different susceptibility to alteration, and/or may affect the function of various other molecules. In one approach, one would generate a three-dimensional structure for a known candidate substance and/or characteristic portion thereof. In some embodiments, this is accomplished by x-ray crystallography, computer modeling, and/or by a combination of these approaches.

In some embodiments, antibodies arc used to ascertain the structure of a candidate substance antagonist. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and/or isolate peptides from banks of chemically- and/or biologically-produced peptides. Selected peptides would then serve as pharmacores. Anti-idiotypes may be generated using methods described herein for producing antibodies, using an antibody as the antigen.

In addition to CXCL12 and/or CXCR4 antagonists initially identified, the inventors contemplate that other sterically similar substances may be formulated to mimic key portions of the structures of CXCL12 and/or CXCR4 antagonists. Such substances, which may include peptidomimetics of peptide modulators, may be used in the same manner as initially-identified CXCL12 and/or CXCR4 antagonists.

Libraries

In some embodiments, libraries of candidate substances may be employed in methods, systems, and/or compositions described herein. The phrase “library of candidate substances,” as used herein, refers to a collection of multiple species of substances that consist of randomly- and/or systematically-selected subunits and/or members. Screening libraries of candidate substances is a rapid and/or efficient way to screen large number of related (and unrelated) compounds for activity. Combinatorial approaches lend themselves to rapid evolution of potential drugs by creation of second, third, and/or fourth generation substances modeled of active, but otherwise undesirable substances.

In certain embodiments, combinatorial libraries (also known as “combinatorial chemical libraries”), small molecule libraries, peptides and/or peptide mimetics, defined chemical entities, oligonucleotides, and/or natural product libraries are screened for activity. In some embodiments, a library of candidate substances may comprise a synthetic combinatorial library (e.g., a combinatorial chemical library), a cellular extract, a bodily fluid (e.g., urine, blood, tears, sweat, and/or saliva), or other mixture of synthetic and/or natural substances (e.g., a library of small molecules and/or a fermentation mixture).

In some embodiments, libraries of candidate substances may include, for example, proteins (e.g., peptides, oligopeptides, and/or amino acids), nucleic acids (e.g., DNA, RNA, oligonucleotides, antisense nucleic acids, ribozymes, and/or peptide nucleic acids), aptamers, carbohydrates (e.g., mono- and/or poly-saccharides), small molecules (e.g., organic small molecules) and/or characteristic portions thereof. Each member of a library may be singular and/or may be a part of a mixture (e.g., a “compressed library”). A library may comprise purified compounds and/or may be “dirty” (i.e., containing a significant quantity of impurities).

In some embodiments, candidate substances may be used in an initial screen in batches of, for example types of substances per reaction. Substances of those batches which show enhancement and/or reduction of the activity being assayed may subsequently be tested individually.

In some embodiments, libraries are acquired from various commercial sources in an effort to “brute force” identify useful substances. In some embodiments, commercially available libraries are obtained from Affymetrix, ArQule, Neose Technologies, Sarco, Ciddco, Oxford Asymmetry, Maybridge, Aldrich, Panlabs, Pharmacopoeia, Sigma, and/or Tripose. A comprehensive review of combinatorial libraries, in particular their construction and/or uses is provided in Dolle et al. (1999, J. of Comb. Chem. 1:235; incorporated herein by reference). Reference is made to combinatorial peptide library protocols (Cabilly, ed., Methods in Molecular Biology, Humana Press, Totowa, N.J., 1998; incorporated herein by reference).

Further references describing combinatorial libraries, their production and/or use include those available from the URL http://www.netsci.org/Science/Combichem/, including The Chemical Generation of Molecular Diversity. Michael R. Pavia, Sphinx Pharmaceuticals, A Division of Eli Lilly (Published July, 1995); Combinatorial Chemistry: A Strategy for the Future—MDL Information Systems discusses the role its Project Library plays in managing diversity libraries (Published July, 1995); Solid Support Combinatorial Chemistry in Lead Discovery and SAR Optimization, Adnan M. M. Mjalli and Barry E. Toyonaga, Ontogen Corporation (Published July, 1995); Non-Peptidic Bradykinin Receptor Antagonists From a Structurally Directed Non-Peptide Library. Sarvajit Chakravarty, Babu J. Mavunkel, Robin Andy, Donald J. Kyle*, Scios Nova Inc. (Published July, 1995); Combinatorial Chemistry Library Design using Pharmacophore Diversity Keith Davies and Clive Briant, Chemical Design Ltd. (Published July, 1995); A Database System for Combinatorial Synthesis Experiments—Craig James and David Weininger, Daylight Chemical Information Systems, Inc. (Published July, 1995); An Information Management Architecture for Combinatorial Chemistry, Keith Davies and Catherine White, Chemical Design Ltd. (Published July, 1995); Novel Software Tools for Addressing Chemical Diversity, R. S. Pearlman, Laboratory for Molecular Graphics and Theoretical Modeling, College of Pharmacy, University of Texas (Published June/July, 1996); Opportunities for Computational Chemists Afforded by the New Strategies in Drug Discovery: An Opinion, Yvonne Connolly Martin, Computer Assisted Molecular Design Project, Abbott Laboratories (Published June/July, 1996); Combinatorial Chemistry and Molecular Diversity Course at the University of Louisville: A Description, Arno F. Spatola, Department of Chemistry, University of Louisville (Published June/July, 1996); Chemically Generated Screening Libraries: Present and Future. Michael R. Pavia, Sphinx Pharmaceuticals, A Division of Eli Lilly (Published June/July, 1996); Chemical Strategies For Introducing Carbohydrate Molecular Diversity Into The Drug Discovery Process. Michael J. Sofia, Transcell Technologies Inc. (Published June/July, 1996); Data Management for Combinatorial Chemistry. Maryjo Zaborowski, Chiron Corporation and Sheila H. DeWitt, Parke-Davis Pharmaceutical Research, Division of Warner-Lambert Company (Published November, 1995); and/or The Impact of High Throughput Organic Synthesis on R&D in Bio-Based Industries, John P. Devlin (Published March, 1996).

Selection protocols for isolating desired members of large libraries are known in the art, as typified by phage display techniques. Such systems, in which diverse peptide sequences are displayed on the surface of filamentous bacteriophage have proven useful for creating libraries of antibody fragments (and nucleotide sequences that encoding them) for in vitro selection and/or amplification of specific antibody fragments that bind a target antigen.

In certain embodiments, alternative library selection technologies include bacteriophage lambda expression systems, which may be screened directly as bacteriophage plaques and/or as colonies of lysogens, as previously described (Huse et al., 1989, Science 246:1275; Caton et al., 1990, Proc. Natl. Acad. Sci. USA. 87; Mullinax et al., 1990, Proc. Natl. Acad. Sci. USA. 87: 8095; and Persson et al., 1991, Proc. Natl. Acad. Sci. USA. 88: 2432; all of which are incorporated herein by reference) and are of use. These expression systems may be used to screen a large number of different members of a library, in the order of about 10⁶ or more.

In some embodiments, screening systems rely, for example, on direct chemical synthesis of library members. One method involves synthesis of peptides on a set of pins and/or rods, such as described in PCT Publication WO 84/03564 (incorporated herein by reference). A similar method involving peptide synthesis on beads, which forms a peptide library in which each bead is an individual library member, is described in U.S. Pat. No. 4,631,211 and a related method is described in PCT Publication WO 92/00091 (both of which are incorporated herein by reference). A significant improvement of bead-based methods involves tagging each bead with a unique identifier tag, such as an oligonucleotide, so as to facilitate identification of the amino acid sequence of each library member. These improved bead-based methods arc described in PCT Publication WO 93/06121 (incorporated herein by reference).

Another chemical synthesis method involves synthesis of arrays of peptides (or peptidomimetics) on a surface in a manner that places each distinct library member (e.g., unique peptide sequence) at a discrete, predefined location in the array. The identity of each library member is determined by its spatial location in the array. Locations in the array where binding interactions between a predetermined molecule (e.g., a receptor) and reactive library members occur is determined, thereby identifying sequences of reactive library members on the basis of spatial location. These methods are described in U.S. Pat. No. 5,143,854; PCT Publications WO 90/15070 and WO 92/10092; Fodor et al., 1991, Science 251: 767; and Dower et al., 1991, Ann. Rep. Med. Chem. 26: 271 (all of which are incorporated herein by reference).

Other systems for generating libraries of polypeptides or nucleotides involve use of cell-free enzymatic machinery for in vitro synthesis of library members. In one method, RNA molecules are selected by alternate rounds of selection against a target ligand and PCR amplification (Tuerk et al., 1990, Science 249: 505; and Ellington et al., 1990, Nature 346: 818; both of which arc incorporated herein by reference). A similar technique may be used to identify DNA sequences which bind a predetermined human transcription factor (Thiesen et al., 1990, Nucleic Acids Res. 18: 3203; Beaudry et al., 1992 Science 257:635; and PCT Publications WO 92/05258 and WO 92/14843; all of which are incorporated herein by reference). In a similar way, in vitro translation may be used to synthesize polypeptides as a method for generating large libraries. These methods which generally comprise stabilized polysome complexes, are described further in PCT Publications WO 88/08453, WO 90/05785, WO 90/07003, WO 91/02076, WO 91/05058, and WO 92/02536 (all of which are incorporated herein by reference). Alternative display systems which are not phage-based, such as those disclosed in PCT Publications WO 95/22625 and WO 95/11922 (both of which are incorporated herein by reference), use polysomes to display polypeptides for selection.

One combinatorial approach in use is based on a strategy involving synthesis of libraries containing a different structure on each particle of a solid phase support (e.g., a bead), interaction of a library with a soluble candidate substance, identification of the support structure (e.g., bead) which interacts with a candidate substance, and determination of the structure carried by the identified support structure (Lam et al., 1991, Nature 354:82; incorporated herein by reference). An alternative or additional approach is sequential release of defined aliquots of candidate substances from the support structure, with subsequent determination of activity in solution, identification of the support structure from which a candidate substance was released, and elucidation of its structure by direct sequencing (Salmon et al., 1993, Proc. Natl. Acad. Sci. USA 90:11708; incorporated herein by reference) and/or by reading its code (Kerr et al., 1993, J. Am. Chem. Soc. 115:2529; Nikolaicv et al., 1993, Pept. Res. 6:161; and Ohlmeyer et al., 1993, Proc. Natl. Acad. Sci. USA 90:10922; all of which are incorporated herein by reference).

In some embodiments, soluble random combinatorial libraries may be synthesized using a simple principle for generation of equimolar mixtures of peptides which was first described by Furka et al. (1988, Xth International Symposium on Medicinal Chemistry, Budapest; 1988, 14th International Congress of Biochemistry, Prague; and 1991, Int. J. Peptide Protein Res. 37:487-493; all of which are incorporated herein by reference). The construction of soluble libraries for iterative screening has been described (Houghten et al., 1991, Nature 354:84; incorporated herein by reference). Lam et al. disclosed the novel and unexpectedly powerful technique of using insoluble random combinatorial libraries. Lam synthesized random combinatorial libraries on solid phase supports, so that each support had a test compound of uniform molecular structure, and screened libraries without prior removal of test compounds from the support by solid phase binding protocols (Lam et al., 1991, Nature 354:82; incorporated herein by reference).

In some embodiments, special libraries called “diversity files” may be used to assess the specificity, reliability, and/or reproducibility of new methods. Diversity files contain a large number of compounds (e.g., 1000 or more small molecules) representative of many classes of compounds that could potentially result in nonspecific detection in an assay. Diversity files are commercially available and/or can be assembled from individual substances that are commercially available.

The present invention provides screening of libraries of candidate substances. Such libraries may be exposed to a test substance, as defined herein, and to relevant assay(s), described in detail below, carried out. Such libraries may be used in any of the in vitro, in cyto, and/or in vivo assay(s) described in detail below.

Screening

In some embodiments, screening for CXCL12 and/or CXCR4 antagonists is employed. In some embodiments, high throughput screening for CXCL12 and/or CXCR4 antagonists is employed. In some embodiments, such screening identifies substances that bind to test substances, as described herein. Large numbers of candidate substances arc immobilized on a solid substrate. Candidate substances arc contacted with a test substance and washed. Bound test substance is then detected by methods well known in the art.

In high throughput assays in accordance with the invention, it is possible to screen up to several thousand candidate substances in a single day. Each well of a microtiter plate can be used to run a separate assay against a selected candidate substance, or, if concentration and/or incubation time effects are to be observed, every 5-10 wells can test a single candidate substance. Thus, a single standard microtiter plate can assay 96 modulators. If 1536 well plates are used, then a single plate can easily assay from about 100- about 1500 different candidate substances. It is possible to assay many plates per day; assay screens for up to about 6,000, 20,000, 50,000, or more than 100,000 different candidate substances are possible using integrated systems in accordance with the invention.

For a solid state reaction, a substance of interest may be bound to a solid state component, directly or indirectly, via covalent and/or non covalent linkage e.g., via a tag. A tag may comprise any of a variety of components. In general, a substance which binds the tag (a tag binder) is fixed to a solid support, and the tagged molecule of interest is attached to the solid support by interaction of the tag and/or the tag binder.

A number of tags and/or tag binders may be used, based upon known molecular interactions well described in the literature. For example, where a tag has a natural binder, for example, biotin, protein A, and/or protein G, it may be used in conjunction with appropriate tag binders (avidin, streptavidin, neutravidin, the Fc region of an immunoglobulin, etc.). Antibodies to molecules with natural binders such as biotin and/or appropriate tag binders are widely available (Sigma Immunochemicals, 1998 catalogue, St. Louis, Mo.).

Similarly, any haptenic and/or antigenic compound may be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are appropriate as tag and/or tag-binder pairs, including but not limited to transferrin, c-kit, viral receptor ligands, cytokine receptors, chemokine receptors, interleukin receptors, immunoglobulin receptors and/or antibodies, the cadherin family, the integrin family, the selectin family, etc. (see, e.g., Pigott et al., The Adhesion Molecule Facts Book I, 1993; incorporated herein by reference). Similarly, toxins and/or venoms; viral epitopes; hormones (e.g., opiates, steroids, etc.); intracellular receptors (e.g., which mediate effects of various small ligands, including steroids, thyroid hormone, retinoids, vitamin D, and/or peptides); drugs; lectins; carbohydrates; nucleic acids (linear and/or cyclic polymer configurations); proteins; phospholipids; and/or antibodies may interact with various cell receptors.

Synthetic polymers, such as polyurethanes, polyesters, polycarbonates, polyureas, polyamides, polyethyleneimines, polyarylene sulfides, polysiloxanes, polyimides, and/or polyacetates may form appropriate tags and/or tag binders. Many other tag/tag binder pairs are useful in assay systems described herein, as would be apparent to one skilled in the art.

Common linkers such as peptides, polyethers, and the like may serve as tags and may include polypeptide sequences, such as poly-Gly sequences of between about 5 and about 200 amino acids. Such flexible linkers are known to persons of skill in the art. For example, poly(ethelyne glycol) linkers are available from Shearwater Polymers, Inc. (Huntsville, Ala.). These linkers optionally have amide linkages, sulfhydryl linkages, and/or heterofunctional linkages.

Tag binders are fixed to solid substrates using any of a variety of methods currently available. Solid substrates are commonly derivatized and/or functionalized by exposing all and/or a portion of the substrate to a chemical reagent which fixes a chemical group to the surface which is reactive with a portion of the tag binder. For example, groups which are suitable for attachment to a longer chain portion include amines, hydroxyl, thiol, and/or carboxyl groups. Aminoalkylsilanes and/or hydroxyalkylsilanes may be used to functionalize a variety of surfaces, such as glass surfaces. The construction of such solid phase biopolymer arrays is well described in the literature (see, e.g., Merrifield, 1963, J. Am. Chem. Soc. 85:2149, describing solid phase synthesis of, e.g., peptides; Geysen et al., 1987, J. Immun. Meth. 102:259, describing synthesis of solid phase components on pins; Frank et al., 1988, Tetrahedron 44:6031, describing synthesis of various peptide sequences on cellulose disks; Fodor et al., 1991, Science, 251:767; Sheldon et al., 1993, Clinical Chemistry 39(4):718; and Kozal et al., 1996, Nature Medicine 2:753; all describing arrays of biopolymers fixed to solid substrates; all of which are incorporated herein by reference). Non-chemical approaches for fixing tag binders to substrates include other common methods, such as heat, cross-linking by UV radiation, and the like.

In Vitro Assays

In vitro assays can often be run quickly and/or in large numbers, thereby increasing the amount of information obtainable in a short period of time. A variety of vessels may be used to run assays, including test tubes, plates, dishes, and/or other surfaces such as dipsticks and/or beads.

The present invention provides in vitro methods for screening for CXCL12 and/or CXCR4 antagonists. For example, in some embodiments, a method generally comprises steps of: (1) providing a test substance (e.g., CXCL12 and/or CXCR4 protein and/or the CXCL12 and/or CXCR4 gene); (2) providing a candidate substance; and (3) measuring and/or detecting the influence of the candidate substance on the test substance.

In general, a test substance is provided and brought directly and/or indirectly into contact with a candidate substance, e.g., in the form of a library. Then, the influence of the candidate substance on the test substance is detected and/or measured. Thereafter, suitable antagonists may be isolated and/or analyzed. For screening libraries, high-throughput assays, which are known to the skilled person, are commercially available, and are described herein.

In some embodiments, in vitro assays comprise binding assays. Binding of a candidate substance to a test substance (e.g., CXCL12 and/or CXCR4 and/or a homolog, portion, variant, mutant, and/or derivative thereof) may, in and of itself, be inhibitory, due to steric, allosteric, and/or charge-charge interactions. The test substance may be free in solution, fixed to a support, and/or expressed in and/or on the surface of a cell. The test substance and/or the candidate substance may be labeled, thereby permitting detection of binding. The test substance is frequently the labeled species, decreasing the chance that labeling will interfere with and/or enhance binding. Competitive binding formats may be performed in which one of the substances is labeled, and one may measure the amount of free label versus bound label to determine the effect on binding.

In some embodiments, binding assays involve exposing CXCL12 and CXCR4 proteins (including homologs, portions, variants, mutants, and/or derivatives thereof) to a candidate substance and detecting binding between CXCL12 and CXCR4 in the presence of a candidate substance. A binding assay may be conducted in vitro (e.g., in a test tube, comprising substantially only the components mentioned; in cell-free extracts; and/or in substantially purified components). Alternatively or additionally, assays may be conducted in cyto and/or in vivo (e.g., within a cell, tissue, organ, and/or organism; described in further detail below).

In some embodiments, an assay for identifying substances that bind to a test substance (e.g., CXCL12 and/or CXCR4 protein, including homologs, portions, variants, mutants, and/or derivatives thereof), which is immobilized on a solid support, with a non-immobilized candidate substance is used to determine whether and/or to what extent the test substance and candidate substance bind to each other. Alternatively, the candidate substance may be immobilized and the test substance non-immobilized. Such assays may be used to identify candidate substances capable of binding to the test substance.

In some embodiments, an antibody that recognizes a test substance (e.g., α-CXCL12 and/or α-CXCR4 antibody) is bound to a solid support (e.g., Protein-A beads). An antibody is contacted with a corresponding antigen, which binds to the immobilized antibody. The resulting complex is then brought into contact with the candidate substance (purified protein, cellular extract, combinatorial library, etc.). If the antibody-bound antigen interacts with the candidate substance, the candidate substance will become indirectly immobilized to the solid support. Presence of the candidate substance on the solid support can be assayed by any standard technique known in the art (including, but not limited to, western blotting). This type of assay is known in the art as an “immunoprecipitation” assay.

In one embodiment, a test substance (e.g., CXCL12 and/or CXCR4 protein, including homologs, portions, variants, mutants, and/or derivatives thereof) is immobilized on beads, such as agarose beads. In one specific embodiment, CXCL12 and/or CXCR4 protein and/or a characteristic portion thereof is expressed as a GST-fusion protein in bacteria, yeast, insect, and/or higher eukaryotic cell line and/or purified from crude cell extracts using glutathione-agarose beads. As a control, binding of the candidate substance, which is not a GST-fusion protein, to the immobilized CXCL12 and/or CXCR4 protein is determined in the absence of CXCL12 and/or CXCR4 protein. Binding of the candidate substance to the immobilized CXCL12 and/or CXCR4 protein is then determined. This type of assay is known in the art as a “GST pulldown” assay. Alternatively or additionally, the candidate substance may be immobilized and the candidate substance non-immobilized.

It is possible to perform this type of assay using different affinity purification systems for immobilizing one of the components, for example Ni-NTA agarose- and/or histidine-tagged components.

Binding of a test substance to a candidate substance may be determined by a variety of methods well-known in the art. For example, the non-immobilized component may be labeled (with for example, a radioactive label, an epitope tag, and/or an enzyme-antibody conjugate). Alternatively, binding may be determined by immunological detection techniques. For example, the reaction mixture may be Western blotted and the blot probed with an antibody that detects the non-immobilized component. Alternatively or additionally, enzyme linked immunosorbent assay (ELISA) may be utilized to assay for binding.

In some embodiments, screening methods in accordance with the present invention comprise: (1) providing a candidate substance; (2) contacting the candidate substance with CXCL12 and CXCR4; and (2) detecting inhibition of binding between CXCL12 and CXCR4.

In some embodiments, screening methods in accordance with the present invention comprise: (1) providing a candidate substance; and (2) contacting the candidate substance with a pre-formed CXCL12-CXCR4 complex; and (3) determining whether the candidate substance affects the CXCL12-CXCR4 complex.

In Cyto Assays

In some embodiments, the present invention provides methods of screening for CXCL12 and/or CXCR4 antagonists wherein a candidate substance is contacted with a cell. The cell can then be assayed for various parameters associated with CXCL12 and/or CXCR4 activity. For example, parameters associated with CXCL12 and/or CXCR4 activity include, but are not limited to, the ability of CXCL12 to bind to CXCR4.

In certain embodiments, cells may be directly assayed for binding between CXCL12 and CXCR4. Immunohistochemical techniques, confocal techniques, and/or other techniques to assess binding arc well known to those of skill in the art. Various cell lines may be utilized for such screening assays, including cells specifically engineered for this purpose. Examples of cells used in screening assays include T cells and/or other lymphocytes. One of skill in the art would understand that the invention disclosed herein contemplates a wide variety of in cyto assays for measuring parameters that correlate with the activity of CXCL12 and/or CXCR4.

Depending on the assay, cell and/or tissue culture may be required. A cell may be examined using any of a number of different physiologic assays, as discussed above for binding between CXCL12 and CXCR4. Alternatively, molecular analysis may be performed, including, but not limited to, western blotting to monitor protein expression and/or test for protein-protein interactions; northern blotting, differential display of RNA, and/or microarray analysis to monitor mRNA expression; kinase assays to monitor phosphorylation; mass spectrometry to monitor other chemical modifications; etc.

The present invention provides new methods for identifying substances that bind to CXCL12 and/or CXCR4 and, therefore, may modulate CXCL12 and/or CXCR4 activity. One in cyto method of identifying substances that bind to CXCL12 and/or CXCR4 is a two-hybrid system assay (Fields et al., 1994, Trends in Genetics 10:286; and Colas et al., 1998, TIBTECH 16:355; both of which are incorporated herein by reference). In this assay, yeast cells express a first fusion protein consisting of a candidate substance according to the invention (e.g., CXCL12 and/or CXCR4 protein and/or a characteristic portion thereof) and a DNA-binding domain of a transcription factor such as Gal4 and/or LexA. Cells additionally contain a reporter gene whose promoter contains binding sites for the corresponding DNA-binding domain. By transforming cells with a vector that expresses a second fusion protein consisting of a candidate substance fused to an activation domain (e.g., from Gal4 and/or herpes simplex virus VP16), expression of the reporter gene may be greatly increased if the candidate substance interacts with the candidate substance. Consequently this assay may be used for screening for substances that modulate an interaction between CXCL12 and/or CXCR4 and any number of candidate substances. In this way, it is possible rapidly to identify novel CXCL12 and/or CXCR4 antagonists.

Another assay is based on solid phase-bound CXCL12 and/or CXCR4 proteins and their interactions with a candidate substance to be tested. Thus, a candidate substance (e.g., CXCL12 and/or CXCR4 protein and/or a characteristic portion thereof) may contain a detectable marker, such as a radioactive, fluorescent, and/or luminescent label. Furthermore, candidate substances can be coupled to other substances which permit indirect detection (e.g., by means of employing an enzyme which uses a chromogenic substrate and/or by means of binding a detectable antibody). Changes in conformation of CXCL12 and/or CXCR4 as a result of an interaction with a candidate substance may be detected, for example, by a change in emission of the detectable marker. Alternatively or additionally, solid phase-bound protein complexes may be analyzed by means of mass spectrometry.

In some embodiments, screening methods may assay CXCL12 and/or CXCR4 activity by monitoring downstream cellular effects of CXCL12 and/or CXCR4 activity. Such effects include, but are not limited to, activation of multiple signaling pathways, resulting in diverse biological outcomes such as migration, adhesion, and/or transcriptional activation. Pathways activated and/or outcomes elicited may differ between CXCR4+ cell types, and these can be monitored and assayed. In some embodiments, G-protein signaling pathways are activated. In some embodiments, G protein-independent pathways are activated. Tyrosine phosphorylation of CXCR4 results in recruitment and activation of the JAK (JAK2 and JAK3)/STAT pathway, while p38 and ERK activation has been shown to be partially dependent on arrestin-3. Following activation, GRK phosphorylation results in recruitment of arrestin 2/3 and subsequent internalization. CXCR4 is also ubiquitinated by AIP4 at the plasma membrane, which results in its sorting to and degradation in lysosomes. However, a portion of the internalized receptor may also recycle back to the plasma membrane.

In some embodiments, CXCL12 and/or CXCR4 levels arc determined by measuring protein and/or mRNA levels. Levels of CXCL12 and/or CXCR4 protein and/or characteristic portions thereof are measured using immunoassays such as western blotting, radioimmune assay, and/or ELISA using antibodies that selectively bind to CXCL12 and/or CXCR4. For measurement of mRNA, amplification (e.g., using PCR, LCR) and/or hybridization assays (e.g., northern hybridization, RNAse protection, dot blotting) may be used. Protein and/or mRNA levels are detected using directly- and/or indirectly-labeled detection agents, e.g., fluorescently and/or radioactively labeled nucleic acids, radioactively and/or enzymatically labeled antibodies, etc. as described herein.

Alternatively or additionally, CXCL12 and/or CXCR4 expression may be measured using a reporter gene system. Such a system may be devised using a CXCL12 and/or CXCR4 gene promoter operably-linked to a reporter gene such as chloramphenicol acetyltransferase, firefly luciferase, bacterial luciferase, O-galactosidase, and/or alkaline phosphatase. Furthermore, CXCL12 and/or CXCR4 may be used as an indirect reporter via attachment to a second reporter such as red and/or green fluorescent protein (see, e.g., Mistili et al., 1997, Nature Biotech. 15:961; incorporated herein by reference). The reporter construct is typically transfected into a cell. After treatment with a candidate substance, the amount of reporter gene transcription, translation, and/or activity is measured according to standard techniques known to those of skill in the art.

In vivo Assays

In vivo assays involve use of various animal models, including transgenic animals that have been engineered to have specific defects and/or carry markers that can be used to measure the ability of a candidate substance to reach and/or affect different cells within an organism. Due to their size, ease of handling, and/or information on their physiology and/or genetic make-up, mice are amenable to use in in vivo assays. However, other animals are suitable as well, including rats, rabbits, hamsters, guinea pigs, gerbils, woodchucks, cats, dogs, sheep, goats, pigs, cows, horses and/or monkeys (including chimpanzees, gibbons, and/or baboons). Assays for CXCL12 and/or CXCR4 antagonists may be conducted using an animal model derived from any of these species and/or other useful species not listed herein.

In such assays, one or more candidate substances are administered to an animal, and the ability of a candidate substance(s) to alter one or more characteristics, as compared to a similar animal not treated with the candidate substance(s), identifies a CXCL12 and/or CXCR4 antagonist. Characteristics may be any of those discussed herein with regard to symptoms associated with an autoimmune disorder (e.g., diabetes) and/or accumulation of T cells and/or stem cells in bone marrow. Onset of diabetes is typically marked by moderate glycosuria and by a non-fasting plasma glucose higher than 250 mg/dl. Diabetic mice are hypoinsulinemic and hyperglucagonemic, indicating a selective destruction of pancreatic islet beta cells. Alternatively or additionally, onset of diabetes may be marked by extreme thirst; frequent urination; sudden vision changes; sugar in urine; fruity, sweet, or wine-like odor on breath; increased appetite; sudden weight loss; drowsiness; lethargy; heavy, labored breathing; stupor; unconsciousness; diabetic ketoacidosis (DKA); and/or hyperosmolar hyperglycemic nonketotic coma (HNKS).

The present invention provides methods of screening for a candidate substance that may treat, stabilize, and/or delay onset of an autoimmune disorder (e.g., diabetes). In some embodiments, a candidate substance comprises a CXCL12 and/or CXCR4 antagonist. Treatment of these animals with candidate substances will involve administration of the candidate substance, in an appropriate form, to the animal. Administration will be by any route that could be utilized for clinical and/or non-clinical purposes, including but not limited to oral, nasal, buccal, and/or topical. Alternatively or additionally, administration may be by intratracheal instillation, bronchial instillation, intradermal, subcutaneous, intramuscular, intraperitoneal, inhalation, and/or intravenous injection. Specifically contemplated routes are systemic intravenous injection, regional administration via blood and/or lymph supply, and/or direct administration to an affected site.

Accordingly, in some embodiments, the invention provides a screening system, including methods and/or compositions, for determining whether a candidate substance is useful for treating, stabilizing, and/or delaying the onset of an autoimmune disorder (e.g., diabetes) in a mammal. In some embodiments, a candidate substance is determined to treat, stabilize, and/or delay the onset of an autoimmune disorder if the substance improves, stabilizes, and/or delays the onset of symptoms associated with the autoimmune disorder.

In some embodiments, screening systems in accordance with the present invention may involve use of the NOD mouse (i.e., an in vivo model system for type I diabetes), as described herein. NOD mice and humans with type I diabetes display similar phenotypes, such as two stages of disease development, T cell-mediated autoimmune disease, destruction of beta cells, etc.

In some embodiments, screening systems in accordance with the present invention may involve use of the BioBreeding/Worcester rat, which develops a spontaneous syndrome resembling human type 1 diabetes mellitus. Salient features include abrupt onset of insulin dependent, ketosis-prone diabetes between 60 days and 120 days of age (Nakooda et al., 1977, Diabetes, 26:100; incorporated herein by reference); lymphocytic insulitis with virtually complete destruction of pancreatic β cells (Seemayer et al., 1982, Am. J. Pathol., 106:237; and Logothetopoulos et al., 1984, Diabetes, 33:33; both of which are incorporated herein by reference); and genetic predisposition and occurrence of hyperglycemia in the majority of inbred BioBreeding/Worcester (BB/Wor) animals (Butler et al., 1983, Can. J. Genet. Cytol., 25:7; incorporated herein by reference).

While assays described below utilize the NOD mouse model and/or the BB/Wor rat model for type I diabetes, one of ordinary skill in the art will readily recognize that any of these assays could be performed using any in vivo model for any autoimmune disease.

In specific embodiments, the present invention provides methods of identifying novel CXCL12 and/or CXCR4 antagonists useful in treating diabetes comprising steps of (1) providing a NOD mouse and/or BB/Wor rat exhibiting symptoms of diabetes, (2) administering a candidate substance to the animal, (3) measuring the effect(s) of the candidate substance on the symptoms of diabetes by using blood or urine glucose level, and/or (4) histology analysis of T cell and/or leukocyte infiltration into islets of or into salivary glands.

In specific embodiments, the present invention provides methods of identifying novel CXCL12 and/or CXCR4 antagonists useful in the treatment of diabetes comprising steps of (1) providing a NOD mouse and/or BB/Wor rat exhibiting symptoms of diabetes, (2) administering a candidate substance to the animal, and (3) assaying for increased mobilization of naïve T cells and/or stem cells from bone marrow to peripheral lymphoid organs (e.g., by measuring changes in percentage of cells and/or number of cells in bone marrow with or without treatment).

In some embodiments, the present invention provides a method of treating diabetes comprising steps of (1) providing a NOD mouse and/or BB/Wor rat exhibiting symptoms of diabetes, (2) administering a candidate substance to the animal, and (3) measuring the effect(s) of the candidate substance on CXCL12 and/or CXCR4 mRNA and/or protein.

In some embodiments, screening methods in accordance with the present invention involve measuring the in vivo interaction between CXCL12 and/or CXCR4 and their natural binding partners. A candidate substance is determined to treat, stabilize, and/or delay the onset of an autoimmune disorder if the substance modulates the interaction between CXCL12 and/or CXCR4 and their natural binding partners. In certain embodiments, a candidate substance is determined to treat, stabilize, and/or delay the onset of an autoimmune disorder if the substance modulates the interaction between CXCL12 and CXCR4. The interaction between CXCL12 and/or CXCR4 and their natural binding partners may be measured using standard methods, which are described herein and in Sambrook et al. (Molecular Cloning: A Laboratory Manual, 3^{rd} ed., Cold Spring Harbor Laboratory Press, Cold Spring Harbor, N.Y., 2001).

In some embodiments, screening methods in accordance with the present invention involve measuring the activity of the signal transduction cascade downstream of CXCL12 and CXCR4 in a cell, tissue, and/or mammal in the presence and/or absence of the candidate substance. A candidate substance is determined to treat, stabilize, and/or delay the onset of an autoimmune disorder if the substance modulates the activity of a downstream signal transduction cascade.

In some embodiments, screening methods in accordance with the present invention involve measuring CXCL12 and/or CXCR4 mRNA and/or protein levels in a cell, tissue, and/or mammal in the presence and/or absence of a candidate substance. A candidate substance is determined to treat, stabilize, and/or delay the onset of an autoimmune disorder if the substance lowers CXCL12 and/or CXCR4 mRNA and/or protein levels. CXCL12 and/or CXCR4 mRNA and/or protein levels may be measured using standard methods, which are described herein and in Sambrook et al. (Molecular Cloning: A Laboratory Manual, 3^{rd} ed., Cold Spring Harbor Laboratory Press, Cold Spring Harbor, N.Y., 2001).

Determining the effectiveness of a compound in vivo may involve a variety of different criteria. Measuring toxicity and/or dose response may be performed in animals in a more meaningful fashion than in in vitro and/or in cyto assays.

### Applications

Compositions, systems, and methods described herein can be useful for identifying patients suffering from or susceptible to an autoimmune disorder (e.g., diabetes) that is associated with elevated levels of CXCL12 in a particular tissue (e.g., bone marrow), and/or treatment and/or diagnosis of such an autoimmune disorder. Subjects and/or patients in accordance with the present invention include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Methods of Identifying a Patient Population

The present invention provides methods of identifying a patient who might be likely to respond to treatment with CXCL12 and/or CXCR4 antagonists, as described herein. The present invention encompasses the recognition that some autoimmune disorders (e.g., diabetes) are associated with elevated levels of CXCL12 in particular tissues (e.g., bone marrow). The present invention encompasses the recognition that identification of patients suffering from or susceptible to an autoimmune disorder (e.g., diabetes) that is associated with elevated levels of CXCL12 in particular tissues (e.g., bone marrow) is desirable because it allows for identification of patients who might be likely to respond to particular therapies (e.g., CXCL12 and/or CXCR4 antagonists).

Thus, in some embodiments, the present invention provides a method comprising steps of: (1) providing a subject suffering from and/or susceptible to an autoimmune disorder, such as diabetes, (2) providing a sample of a particular test tissue sample from the subject (e.g., bone marrow and/or blood), (3) assaying levels of CXCL12 in the test sample, (4) identifying patients with elevated levels of CXCL12 in the test sample, and (4) administering to these patients a level of CXCL12 and/or CXCR4 antagonist that is sufficient to treat, alleviate, ameliorate, relieve, delay onset of, inhibit progression of, reduce severity of, and/or reduce incidence of one or more symptoms or features of the autoimmune disorder.

Acquisition of Test Sample

In accordance with the present invention, test samples can be obtained (e.g., harvested from a subject) using any method known in the art. In some embodiments, test samples include, but are not limited to, bone marrow; blood; blood cells (e.g., white blood cells, red blood cells, etc.); ascites; tissue or fine needle biopsy samples; cell-containing body fluids; free floating nucleic acids; sputum; urine; cerebrospinal fluid, peritoneal fluid; pleural fluid; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a sample is or comprises cells obtained from a patient. Cells may be, for example, from blood, bone marrow, and/or from tissue derived from solid organs, such as brain, spleen, bone, heart, vascular, lung, kidney, liver, pituitary, endocrine glands, lymph node, dispersed primary cells, tumor cells, etc. In some embodiments, a sample may be a body fluid, including, but not limited to, blood fluids, lymph, ascitic fluids, gynecological fluids, urine, etc. Samples may be obtained from a subject by any of a wide variety of methods including biopsy (e.g., fine needle aspiration or tissue biopsy), surgery, collection of body fluid (e.g., blood, lymph, etc.), etc. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc.

In some embodiments, a test sample is bone marrow. In some embodiments, bone marrow samples can be collected from cavities in hipbones (e.g., from the crest of the ilium), sternum, or other bones of a subject. Bone marrow harvesting is typically performed under general anesthesia. In some embodiments, bone marrow is collected from a test subject and from a reference subject (e.g., a non-diabetic individual) which can provide a negative control for subsequent analysis of the test bone marrow sample.

In some embodiments, a test sample is blood. In some embodiments, blood is harvested directly from a vein or artery of a subject using standard techniques known in the art of phlebotomy. In some embodiments, blood may be harvested from a vein or artery located in the antecubital area of the arm, back of hand, side of wrist, foot, ankle, etc. In some embodiments, blood is harvested from a small prick in the tip of a finger. In some embodiments, blood is stored until subsequent analysis in a tube. In some embodiments, blood can be stored in a culture tube or coagulation tube. In some embodiments, blood can be stored in a tube containing one or more of the following: gel separator, clot activator, sodium heparin, lithium heparin, EDTA, acid citrate dextrose, oxalate/fluoride, or any other additive that is used to preserve the blood sample until it is analyzed.

In some embodiments, test samples are subjected to one or more processing steps before test samples are subjected to analysis (e.g., measurement of CXCL12 levels). In some embodiments, test samples are not subjected to one or more processing steps before test samples are subjected to analysis (e.g., measurement of CXCL12 levels), but instead are directly subjected to analysis. To give but a few examples, processing steps may include centrifugation, filtration, addition of particular chemical entities (e.g., salts, fluorescent moieties, buffering agents, detergents, substance to be used as internal control, etc.), adjustment of pH, isolation of subpopulations within a sample (e.g., centrifugation of blood and subsequent isolation of plasma), heating or cooling, etc.

In some embodiments, test samples (including both unprocessed and processed samples) are stored for a period of time before they are subjected to analysis. In certain embodiments, test samples may be frozen before they are subjected to analysis. Test samples may be frozen at temperatures of about 4° C., about 0° C., about −10° C., about −20° C., about −30° C., about −40° C., about −50° C., about −60° C., about −70° C., about −80° C., about −90° C., about −100° C., or colder.

Test samples can be obtained from a patient using any method known to one of ordinary skill in the art. One of ordinary skill in the art will readily recognize that the descriptions above are not a comprehensive listing of methods for obtaining test samples from a patient, but instead, represent exemplary methods for obtaining test samples from a patient.

Analysis of Test Sample

Assaying levels of CXCL12 in a particular test tissue (e.g., bone marrow, blood, etc.) can be accomplished by any method known to one of ordinary skill in the art. In some embodiments, CXCL12 protein levels in a particular test sample can be measured directly and compared to levels in a reference sample. In some embodiments, CXCL12 protein levels in a particular test sample can be measured indirectly and compared to levels in a reference sample.

In some embodiments, immunoassays can be used to quantify levels of CXCL12 in a test sample, and many such immunoassay techniques are known in the art. For example, antibodies which recognize CXCL12 can be utilized in such immunoassays. The invention is not limited to a particular assay procedure, and therefore is intended to include both homogeneous and heterogeneous procedures. Exemplary immunoassays which can be conducted according to the invention include, but are not limited to, western blotting, fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety or label group can be attached to an antibody to be used in such methods and is selected so as to meet the needs of various uses of the method which are often dictated by the availability of assay equipment and compatible immunoassay procedures. General techniques to be used in performing various immunoassays noted above are known to those of ordinary skill in the art.

In some embodiments, flow-cytometric methods can be used to quantify levels of CXCL12 in a test sample. Flow cytometry allows for identification of proteins on a cell surface as well as intracellular proteins using fluorochrome labeled, protein-specific antibodies or non-labeled antibodies in combination with fluorochrome labeled secondary antibodies. General techniques to be used in performing flow cytometric assays noted above are known to those of ordinary skill in the art.

In some embodiments, CXCL12 levels in a test sample can be quantified by 2D gel-electrophoresis and/or mass spectrometry. Determination of the protein nature, sequence, molecular mass as well charge can be achieved in one detection step. Mass spectrometry can be performed with methods known to those with skills in the art, such as MALDI, TOF, or combinations of these.

In some embodiments, CXCL12 RNA levels are correlative with CXCL12 protein levels. In some embodiments, CXCL12 RNA levels arc measured by northern blotting. In some embodiments, CXCL12 RNA levels are measured by quantitative RT-PCR analysis. In some embodiments, CXCL12 RNA levels are measured using cDNA or oligonucleotide arrays, also known as microarrays (i.e. “GeneChips”), which can provide a method of rapidly and efficiently measuring expression of a large number of genes. In some embodiments, CXCL12 RNA levels can be measured by cytokine microarray expression analysis. In general, when measuring CXCL12 RNA levels, an RNA sample is obtained from a test subject and from a reference subject, and levels of CXCL12 RNA from the two samples are compared to one another (e.g., by northern blotting, quantitative RT-PCR analysis, microarray chip, etc.).

In some embodiments, in vivo imaging (e.g., immunohistochemical staining) may be used to qualitatively determine CXCL12 levels in a test sample. For immunohistochemical staining, a tissue sample is obtained and is typically subjected to high concentrations of detergent and/or proteolytic hydrolysis (e.g., employing such agents as protease K or pepsin). In certain embodiments, it may be desirable to isolate the nuclear fraction from sample cells and detect levels of the marker polypeptide in the nuclear fraction. Test samples arc typically fixed by treatment with a reagent such as formalin, formaldehyde, glutaraldehyde, methanol, or the like. Test samples are then incubated with an antibody with binding specificity for the marker polypeptide. The antibody may be conjugated to a label for subsequent detection of binding. Test samples are incubated for a time sufficient for formation of immunocomplexes. Binding of the antibody is then detected by virtue of a label conjugated to the antibody. Where the antibody is unlabelled, a second labeled antibody may be employed, e.g., an antibody specific for the isotype of the anti-marker polypeptide antibody. Examples of labels which may be employed include radionuclide, fluorescence, chemiluminescence, and enzyme labels.

In some embodiments, levels of CXCL12 in a particular test tissue (e.g., bone marrow) may be assayed indirectly by measuring levels of CXCL12 in a different tissue (e.g., blood). Generally, levels of CXCL12 in one test tissue correlate with levels of CXCL12 in a different test tissue. To give but one example, the present invention encompasses the recognition that assaying levels of CXCL12 and/or CXCR4 in blood can correlate with levels of CXCL12 and/or CXCR4 in bone marrow. Alternatively or additionally, elevated levels in blood could lead accumulation of naïve T cells and/or stem cells in blood, thereby reducing their level in other lymphoid organs (e.g., spleen and lymph nodes).

For example, in such methods as those described above, blood can be obtained from a test subject and from a reference subject and levels of CXCL12 RNA and/or protein in the two samples can be compared to one another. This may be desirable because it is much easier and less dangerous for medical professional to obtain a blood sample than a bone marrow sample.

In some embodiments, levels of CXCL12 in a particular test tissue may be assayed indirectly by monitoring levels of CXCL12 activity in a test sample. For example, levels of CXCL12 in a particular test tissue may be assayed using in vitro using cell migration assays. Cell migration assays are well-known and can be designed and carried out in any way determined by one of ordinary skill in the art. For example, cells expressing CXCR4 on their surfaces may be exposed to a sample (e.g., from a subject's bone marrow, blood, etc.), and the responsiveness of cells to the sample (e.g., rate of migration toward the sample, distance migrated, etc.) can serve as a measure of CXCL12 levels in the sample.

In some embodiments, CXCL12 levels in a test sample may be substantially equal to CXCL12 levels in the reference sample. In some embodiments, CXCL12 levels in a test sample may be substantially increased relative to the reference sample. In some embodiments, CXCL12 levels in a test sample may be increased relative to the reference sample by approximately 10%, approximately 20%, approximately 30%, approximately 40%, approximately 50%, approximately 60%, approximately 70%, approximately 80%, approximately 90%, approximately 100%, approximately 200%, approximately 300%, approximately 400%, approximately 500%, approximately 1000%, approximately 5000%, or more. In some embodiments, CXCL12 levels in a test sample may be one standard deviation higher than CXCL12 levels in the reference sample. In some embodiments, CXCL12 levels in a test sample may be two standard deviations higher than CXCL12 levels in the reference sample. In some embodiments, CXCL12 levels in a test sample may be more than two standard deviations higher than CXCL12 levels in the reference sample. In some embodiments, the difference between CXCL12 levels in a test sample and CXCL12 levels in the reference sample may be statistically significant. In some embodiments, the difference between CXCL12 levels in a test sample and CXCL12 levels in the reference sample may not be statistically significant.

In some embodiments, a relationship is determined between values of an indicator for a subject and the likelihood that a subject will exhibit a favorable response. Results of an evaluation can be expressed in terms of the probability (ranging from 0% to 100%) that a subject having a particular value for CXCL12 levels (e.g., a value for CXCL12 levels that falls within a particular range) will exhibit a favorable response to a CXCL12 and/or CXCR4 antagonist.

The present invention encompasses the recognition that patients exhibiting elevated levels of CXCL12 in particular tissues (e.g., bone marrow) might be likely to respond to therapies involving CXCL12 and/or CXCR4 antagonists. En some embodiments, patients exhibiting elevated levels of CXCL12 in particular tissues (e.g., bone marrow) may display symptoms of an autoimmune disorder (e.g., diabetes). In some embodiments, patients exhibiting elevated levels of CXCL12 in particular tissues may not display symptoms of an autoimmune disorder. Regardless of whether the patient displays symptoms of an autoimmune disorder or not, once patients have been identified with elevated levels of CXCL12 in particular tissues, any substance that behaves as a CXCL12 and/or CXCR4 antagonist (e.g., AMD3100 and/or any other CXCL12 and/or CXCR4 antagonist as described herein) may be administered to a patient. In general, a CXCL12 and/or CXCR4 antagonist is administered in an amount that is sufficient to treat, alleviate, ameliorate, relieve, delay onset of, inhibit progression of, reduce severity of, and/or reduce incidence of one or more symptoms or features of an autoimmune disorder (e.g., diabetes).

According to certain methods, a sample is obtained from a subject who is suffering from and/or susceptible to an autoimmune disorder. The likelihood that the subject might respond favorably to a CXCL12 and/or CXCR4 antagonist is evaluated and is used as a basis on which to determine whether the subject is a suitable candidate for initiating or continuing treatment with a CXCL12 and/or CXCR4 antagonist or for enrolling in or remaining in a clinical trial of a CXCL12 and/or CXCR4 antagonist. For example, if the likelihood is greater than a predetermined value, then the subject may be considered a suitable candidate for initiating or continuing treatment with the CXCL12 and/or CXCR4 antagonist. If the likelihood is less than a predetermined value, then the subject may be considered not suitable as a candidate. One of ordinary skill in the art will recognize that a variety of factors may be considered in determining whether a subject is a suitable candidate for therapy with a CXCL12 and/or CXCR4 antagonist. For example, the subject's response to other therapies, or the results of tests to evaluate the likelihood that the subject will respond to other therapies may be considered as may the side effect profile of the CXCL12 and/or CXCR4 antagonist or of any available alternative therapy.

In some embodiments, methods described above may be employed as follow-up to treatment, e.g., quantification of levels of marker polypeptides may be indicative of effectiveness of current or previously employed therapies for a particular autoimmune disorder (e.g., diabetes) being treated as well as the effect of these therapies upon patient prognosis.

Methods of Treatment

The present invention provides methods of treating and/or diagnosing a patient who is suffering from and/or is susceptible to an autoimmune disorder (e.g., diabetes). The present invention encompasses the recognition that some autoimmune disorders (e.g., diabetes) are associated with elevated levels of CXCL12 in particular tissues (e.g., bone marrow, blood, etc.). The present invention encompasses the recognition that autoimmune disorders that are associated with elevated levels of CXCL12 in particular tissues may be treated with CXCL12 and/or CXCR4 antagonists.

Thus, in some embodiments, the present invention provides a method comprising steps of: (1) providing a subject suffering from and/or susceptible to an autoimmune disorder, such as diabetes, (2) administering a level of CXCL12 and/or CXCR4 antagonist that is sufficient to treat, alleviate, ameliorate, relieve, delay onset of, inhibit progression of, reduce severity of, and/or reduce incidence of one or more symptoms or features of the autoimmune disorder.

The present invention provides methods of identifying patients who are suffering from an autoimmune disorder, but do not display symptoms of the autoimmune disorder. For example, the present inventors have discovered that CXCR12 levels are elevated in 4-5 week old NOD mice, which is long before the usual onset of diabetes in NOD mice. Thus, the present invention provides methods of predicting whether a subject may or may not develop a particular autoimmune disorder (e.g., diabetes).

Thus, in some embodiments, the present invention provides a method comprising steps of: (1 ) providing a subject, (2) measuring levels of CXCL12 and/or CXCR4 in particular tissues (e.g., bone marrow, blood, etc.) in order to identify subjects suffering from and/or susceptible to an autoimmune disorder, such as diabetes, (3) administering a level of CXCL12 and/or CXCR4 antagonist that is sufficient to treat, alleviate, ameliorate, relieve, delay onset of, inhibit progression of, reduce severity of, and/or reduce incidence of one or more symptoms or features of the autoimmune disorder.

Any of the methods of treatment and/or diagnosis described herein may also involve administration of a CXCL12 and/or CXCR4 antagonist in combination with other treatments for the autoimmune disorder. Combination therapies arc described in further detail in the following section.

Administration

In some embodiments, a therapeutically effective amount of a composition in accordance with the invention is delivered to a subject and/or organism prior to, simultaneously with, and/or after diagnosis with a disease, disorder, and/or condition (e.g., an autoimmune disease, such as type I diabetes). In some embodiments, a therapeutic amount of a composition in accordance with the invention is delivered to a patient and/or organism prior to, simultaneously with, and/or after onset of symptoms of a disease, disorder, and/or condition. In some embodiments, the amount of a composition in accordance with the invention is sufficient to treat, alleviate, ameliorate, relieve, delay onset of, inhibit progression of, reduce severity of, and/or reduce incidence of one or more symptoms or features of the disease, disorder, and/or condition.

Compositions in accordance with the present invention may be administered using any amount and any route of administration effective for treatment. The exact amount required will vary from subject to subject, depending on the species, age, and general condition of the subject, the severity of the infection, the particular composition, its mode of administration, its mode of activity, and the like. Compositions in accordance with the invention are typically formulated in dosage unit form for ease of administration and uniformity of dosage. It will be understood, however, that the total daily usage of the compositions in accordance with the present invention will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

Pharmaceutical compositions in accordance with the present invention may be administered by any route. In some embodiments, pharmaceutical compositions in accordance with the present invention are administered by a variety of routes, including oral, intravenous, intramuscular, intra-artcrial, intramedullary, intrathecal, subcutaneous, intraventricular, transdermal, interdermal, rectal, intravaginal, intraperitoneal, topical (as by powders, ointments, creams, and/or drops), transdermal, mucosal, nasal, buccal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In some embodiments, compositions in accordance with the invention are administered parenterally. In some embodiments, compositions in accordance with the invention are administered intravenously. In some embodiments, compositions in accordance with the invention are administered orally.

In general the most appropriate route of administration will depend upon a variety of factors including the nature of the agent (e.g., its stability in the environment of the gastrointestinal tract), the condition of the subject (e.g., whether the subject is able to tolerate oral administration), etc. At present the oral and/or nasal spray and/or aerosol route is most commonly used to deliver therapeutic agents directly to the lungs and/or respiratory system. However, the invention encompasses the delivery of a pharmaceutical composition by any appropriate route taking into consideration likely advances in the sciences of drug delivery.

In certain embodiments, a therapeutic agent may be administered in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

It will be appreciated that therapeutic agents and pharmaceutical compositions in accordance with the present invention can be employed in combination therapies. In some embodiments, the present invention encompasses “therapeutic cocktails” comprising compositions in accordance with the invention. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose. For example, AMD3100 may be administered with another agent that negatively affects the interaction between CXCL12 and CXCR4. To give another example, an agent that negatively affects the interaction between CXCL12 and CXCR4 (e.g., AMD3100) may be administered with another agent that is useful in the treatment of autoimmune disorders (e.g., insulin, cyclosporine, anti-CD3 antibodies, etc.). In some embodiments, the therapies employed may achieve different effects (e.g., control of any adverse side effects).

Pharmaceutical compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. It will further be appreciated that therapeutically active agents utilized in combination may be administered together in a single composition or administered separately in different compositions. Compositions can be administered concurrently with, prior to, or subsequent to, one or more other desired therapeutics or medical procedures. In general, each agent will be administered at a dose and/or on a time schedule determined for that agent. In general, it is expected that agents utilized in combination with be utilized at levels that do not exceed the levels at which they are utilized individually. In some embodiments, the levels utilized in combination will be lower than those utilized individually. Additionally, the invention encompasses delivery of pharmaceutical compositions in combination with agents that may improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an agent in accordance with the invention may be administered concurrently with another therapeutic agent used to treat the same disorder), and/or they may achieve different effects (e.g., control of any adverse side effects). In some embodiments, compositions in accordance with the invention are administered with a second therapeutic agent that is approved by the U.S. Food and Drug Administration.

In some embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of an autoimmune disorder. For example, various agents which inhibit inflammation (e.g., steroids) can be used to treat autoimmune disorders in general. In some embodiments, compositions in accordance with the invention may be administered in combination with agents which inhibit inflammation (e.g., steroids) in order to treat autoimmunc disorders.

In specific embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of diabetes. For example, compositions in accordance with the invention may be administered in combination with traditional diabetes therapies including, but not limited to, insulin administration.

In specific embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of rheumatoid arthritis. For example, compositions in accordance with the invention may be administered in combination with soluble TNF receptor, anti-TNFα receptor, analgesics, non-steroidal anti-inflammatory agents (NSAIDs), and/or other agents useful for treatment of rheumatoid arthritis.

In specific embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of Crohn's disease. For example, compositions in accordance with the invention may be administered in combination with anti-TNFα receptor and/or other agents useful for treatment of Crohn's disease.

In specific embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of multiple sclerosis. For example, compositions in accordance with the invention may be administered in combination with interferon β-1b, interferon β-1a, and/or other agents useful for treatment of multiple sclerosis.

In specific embodiments, compositions in accordance with the invention may be administered in combination with any therapeutic agent or therapeutic regimen that is useful to treat, alleviate, ameliorate, relieve, delay onset of, inhibit progression of, reduce severity of, and/or reduce incidence of one or more symptoms or features of granulomatous disease and/or osteoporosis. For example, compositions in accordance with the invention may be administered in combination with interferon β-1b and/or other agents useful for treatment of granulomatous disease and/or osteoporosis.

One of ordinary skill in the art will understand that the examples presented above are not meant to be limiting. The principles presented in the examples above can be generally applied to any combination therapies for treatment of autoimmune disease.

Pharmaceutical Compositions

The present invention provides novel agents useful for the treatment of autoimmune disorders (e.g., diabetes). In some embodiments, the present invention provides for pharmaceutical compositions comprising agents as described herein and one or more pharmaceutically acceptable excipients. Such pharmaceutical compositions may optionally comprise one or more additional therapeutically-active substances. In accordance with some embodiments, a method of administering a pharmaceutical composition comprising compositions in accordance with the invention to a patient in need thereof is provided. In some embodiments, compositions in accordance with the invention are administered to humans. For the purposes of the present invention, the phrase “active ingredient” generally refers to an agent in accordance with the invention that is useful in the treatment of an autoimmune disorder (e.g., diabetes).

Although the descriptions of pharmaceutical compositions provided herein are principally directed to pharmaceutical compositions which arc suitable for administration to humans, it will be understood by the skilled artisan that such compositions are generally suitable for administration to animals of all sorts. Modification of pharmaceutical compositions suitable for administration to humans in order to render compositions suitable for administration to various animals is well understood, and the ordinarily skilled veterinary pharmacologist can design and/or perform such modification with merely ordinary, if any, experimentation. Patients to which administration of pharmaceutical compositions in accordance with the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Formulations of pharmaceutical compositions described herein may be prepared by any method known or hereafter developed in the art of pharmaceutics. In general, such preparatory methods include the step of bringing an active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of a pharmaceutical composition comprising a predetermined amount of active ingredient. The amount of active ingredient is generally equal to the dosage of active ingredient which would be administered to a subject and/or a convenient fraction of such a dosage such as, for example, one-half or one-third of such a dosage.

Relative amounts of active ingredient, pharmaceutically acceptable excipient(s), and/or any additional ingredients in a pharmaceutical composition in accordance with the invention will vary, depending upon the identity, size, and/or condition of the subject treated and further depending upon the route by which the composition is to be administered. By way of example, a composition may comprise between 0.1% and 100% (w/w) active ingredient.

Pharmaceutical formulations in accordance with the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of a pharmaceutical composition, its use is contemplated to be within the scope of this invention.

In some embodiments, the pharmaceutically acceptable excipient is at least 95%, at least 96%, at least 97%, at least 98%, at least 99%, or about 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in formulations in accordance with the invention. Excipients such as cocoa butter and suppository waxes, coloring agents, coating agents, sweetening, flavoring, and perfuming agents can be present in a composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [Tween 20], polyoxyethylene sorbitan [Tween 60], polyoxyethylene sorbitan monoolcate [Tween 80], sorbitan monopalmitatc [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, polyethoxylated castor oil, polyoxymethylene stcaratc, and Solutol), sucrose fatty acid esters, polyethylene glycol fatty acid esters (e.g., Cremophor), polyoxyethylene ethers, (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Vccgum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl parabcn, methyl paraben, ethyl paraben, propyl paraben, benzoic acid, hydroxybenzoic acid, potassium benzoate, potassium sorbatc, sodium benzoate, sodium propionatc, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but arc not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristatc, jojoba, kukui nut, lavandin, lavender, lemon, litsca cubcba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but arc not limited to, butyl stcaratc, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to active ingredient(s), liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and perfuming agents. In certain embodiments for parenteral administration, an active ingredient can be mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is oftcn desirable to slow absorption of a drug from subcutaneous or intramuscular injection. This may be accomplished by the use of a liquid suspension of crystalline or amorphous material with poor water solubility. The rate of absorption of a drug then depends upon its rate of dissolution which, in turn, may depend upon crystal size and crystalline form. Alternatively, delayed absorption of a parenterally administered drug form is accomplished by dissolving or suspending a drug in an oil vehicle.

Compositions for rectal or vaginal administration are typically suppositories which can be prepared by mixing active ingredients in accordance with the invention with suitable non-irritating excipients such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinonc, sucrose, and acacia, (c) humcctants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and/or (i) lubricants such as talc, calcium stearate, magnesium stearate, solid polyethylene glycols, sodium lauryl sulfate, and mixtures thereof. In the case of capsules, tablets and pills, a dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

Active ingredients can be in micro-encapsulated form with one or more excipients as noted above. Solid dosage forms of tablets, dragccs, capsules, pills, and granules can be prepared with coatings and shells such as enteric coatings, release controlling coatings and other coatings well known in the pharmaceutical formulating art. In such solid dosage forms an active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, dosage forms may comprise buffering agents. They may optionally comprise opacifying agents and can be of a composition that they release active ingredient(s) only, or preferentially, in a certain part of the intestinal tract, optionally, in a delayed manner. Examples of embedding compositions which can be used include polymeric substances and waxes.

Dosage forms for topical and/or transdermal administration of a composition of this invention may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates use of transdermal patches, which often have an added advantage of providing controlled delivery of an active ingredient to the body. Such dosage forms may be prepared, for example, by dissolving and/or dispensing active ingredient in the proper medium. Alternatively or additionally, rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT Publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices arc described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis arc suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of any of the excipients and/or additional ingredients described herein.

A pharmaceutical composition in accordance with the invention may be prepared, packaged, and/or sold in a formulation suitable for pulmonary administration via the buccal cavity. Such a formulation may comprise dry particles which comprise active ingredient and which have a diameter in the range from about 0.5 μm to about 7 μm or from about 1 μm to about 6 μm. Such compositions are conveniently in the form of dry powders for administration using a device comprising a dry powder reservoir to which a stream of propellant may be directed to disperse the powder and/or using a self propelling solvent/powder dispensing container such as a device comprising active ingredient dissolved and/or suspended in a low-boiling propellant in a sealed container. Such powders comprise particles wherein at least 98% of the particles by weight have a diameter greater than 0.5 μm and at least 95% of the particles by number have a diameter less than 7 μm. Alternatively, at least 95% of the particles by weight have a diameter greater than 1 μm and at least 90% of the particles by number have a diameter less than 6 μm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of a composition. A propellant may further comprise additional ingredients such as a liquid non-ionic and/or solid anionic surfactant and/or a solid diluent (which may have a particle size of the same order as particles comprising active ingredient).

Pharmaceutical compositions in accordance with the invention formulated for pulmonary delivery may provide active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 μm to about 200 μm.

Formulations described herein as being useful for pulmonary delivery are useful for intranasal delivery of a pharmaceutical composition in accordance with the invention. Another formulation suitable for intranasal administration is a coarse powder comprising active ingredient and having an average particle from about 0.2 μm to 500 μm. Such a formulation is administered in the manner in which snuff is taken, i.e., by rapid inhalation through the nasal passage from a container of the powder held close to the nares.

Formulations suitable for nasal administration may, for example, comprise from about as little as 0.1% (wiw) and as much as 100% (w/w) of active ingredient, and may comprise one or more of any of the excipients and/or additional ingredients described herein. A pharmaceutical composition in accordance with the invention may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of any of the excipients and/or additional ingredients described herein. Alternately, formulations suitable for buccal administration may comprise a powder and/or an aerosolized and/or atomized solution and/or suspension comprising active ingredient. Such powdered, aerosolized, and/or aerosolized formulations; when dispersed, may have an average particle and/or droplet size in the range from about 0.1 μm to about 200 μm, and may further comprise one or more of any of the excipients and/or additional ingredients described herein.

A pharmaceutical composition in accordance with the invention may be prepared, packaged, and/or sold in a formulation suitable for ophthalmic administration. Such formulations may, for example, be in the form of cyc drops including, for example, a 0.1%11.0% (w/w) solution and/or suspension of active ingredient in an aqueous or oily liquid excipient. Such drops may further comprise buffering agents, salts, and/or one or more of any of the excipients and/or additional ingredients described herein. Other opthalmically-administrable formulations which are useful include those which comprise active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

Kits

The invention provides a variety of kits comprising one or more of any of the compositions in accordance with the invention. For example, the invention provides kits comprising a composition in accordance with the invention and instructions for use. A kit may comprise multiple different compositions. A kit may comprise any of a number of additional components or reagents in any combination. All of the various combinations are not set forth explicitly but each combination is included in the scope of the invention.

According to certain embodiments, a kit may include, for example, (i) a CXCL12 and/or CXCR4 antagonist; (ii) instructions for administering the CXCL12 and/or CXCR4 antagonist to a subject in need thereof.

According to certain embodiments, a kit may be provided which includes materials useful for identifying and/or screening for novel CXCL12 and/or CXCR4 antagonists. Such a kit may include, for example, (i) a library of candidate substances; (ii) a CXCL12 and/or CXCR4 antagonist that may serve as a positive control; (iii) a substance that may serve as a negative control. In some embodiments, the CXCL12 and/or CXCR4 antagonist that may serve as a positive control may comprise a substance that is already known to have activity as a CXCL12 and/or CXCR4 antagonist. In some embodiments, the CXCL12 and/or CXCR4 antagonist that may serve as a positive control may already be known to treat a particular autoimmune disorder (e.g., diabetes). In some embodiments, a substance that may serve as a negative control may be a substance that is already known not to have activity as a CXCL12 and/or CXCR4 antagonist. In some embodiments, the substance that may serve as a negative control may already be known not to treat a particular autoimmune disorder (e.g., diabetes).

According to certain embodiments, a kit may be provided which includes materials useful for identifying and/or screening for patients who may be likely to respond to treatment with CXCL12 and/or CXCR4 antagonists. Such a kit may include, for example, (i) equipment suitable for obtaining a bone marrow and/or blood sample from a subject; (ii) an antibody that recognizes CXCL12 in western blotting and/or ELISA assays; (iii) CXCL12 protein that may serve as a positive control for western blotting and/or ELISA assays; (iv) a reference bone marrow and/or blood sample (e.g., samples from non-diabetic individuals).

Kits typically include instructions for use of compositions in accordance with the invention. Instructions may, for example, comprise protocols and/or describe conditions for production of compositions, administration of compositions to a subject in need thereof, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed.

### Exemplification

### Example 1

### Elevated CXCL12 Expression in the Bone Marrow of NOD Mice Promotes Development of Diabetes by Altering T Cell Trafficking and Stem Cell Mobilization

### Materials and Methods

Mice

NOD mice were obtained from Taconic Farms. Female NOD mice were used in all the studies and monitored for diabetes by checking urine glucose levels at least two times per week, starting around 12 weeks of age. Mice were considered diabetic when urinary glucose level reached 500 mg/dl, as measured with Diastix (Bayer Diagnostics). Diabetic mice also often showed polydipsia, polyuria and weight loss. Approximately 95% of untreated NOD female mice became diabetic by 7 months of age. BALB/c mice were purchased from Jackson Laboratory. EA16NOD mice were obtained from Drs. Diane Mathis and Christopher Benoist. Cxcr4^{fif} Lck-Cre mice and littermate Cxcr4^{-if} Lck-Cre were on the mixed 129/C57BL/6 background and were generated by breeding Cxcr4^{+if} Lck-Cre mice. All mice were kept under specific pathogen free facilities. For AMD3100 treatment, prediabetic NOD mice were given AMD3100 (5 mg/kg) subcutaneously daily for the indicated lengths of time. For analysis of T cell distribution, mice were sacrificed at 2 hours after the last AMD3100 injection.

Antibodies and Flow Cytometry

Single-cell suspensions were prepared from the spleen, lymph nodes (e.g., superficial cervical, brachial, axillary, inguinal, mesenteric, and pancreas-draining lymph nodes), and bone marrow (e.g., obtained by flushing two femurs and two tibias with cold RPMI 1640 medium containing 5% fetal calf serum [FCS]). All staining antibodies were purchased from BDbioscience, except anti-Foxp3, lineage markers (Lin), and c-Kit and Sea-1, which were from E-bioscience, Stem Cell Technologies, and Biolegend, respectively. Cells were stained in the presence of 2.5 μg/ml anti-FcR antibody in PBS containing 0.1% BSA and 0.1% NaN₃ and analyzed on a FACScaliber or a LSR II or a FACSAria, collecting 10,000 to 1,000,000 live cells per sample. Analyses were carried out with Flowjo software. For analysis of LSK cells, 10% of total cells with low level of lineage marker expression were gated for further analysis of Sca-1 versus c-Kit expression profiles. Intracellular staining of Foxp3 and Ki67 was performed according to the manufacturer's instruction.

Immunohistochemistry

Pancreata were fixed in 10% formalin, embedded in paraffin, and sectioned. Paraffin-embedded tissue sections were stained with hacmatoxylin and eosin, and parallel sections were stained with polyclonal guinea pig anti-insulin (Zymed) or anti-glucagon antibodies (Linco Research Inc.), followed by incubation with ABC systems Kit (Vector laboratories). For destructive insulitis (loss of insulin staining), insulin-stained sections of pancreata were matched to serial sections stained for glucagon. Insulitis is defined as presence of infiltrating lymphocytes in islets, regardless of whether they stained positive for insulin or not.

Chemokine Gene Expression

RNAs were isolated from bone marrow using Trizol (Invitrogen) and an RNEasy MiniPrep kit (QIAGEN). RNA was reverse transcribed using the Ampho-Labeling kit (Superarray, Frederick, Md.), and the cDNA was labeled with biotin-16-dUTP (Roche). Cytokine expression was assessed using an Oligo GEArray® Mouse Chemokines & Receptors Microarray (Superarray, Frederick, Md.). Real-time PCR for CXCL12, CCL19, GAPDH, and HPRT transcripts was performed with the probes and master mixture kit from Applied Biosystems.

Homing of T Cells

T cells were purified from lymph nodes and spleens from 12-16 week old NOD and BALB/c mice by negative depletion using a MACS and staining with biotinylated antibodies specific for CD11b, CD11c, and B200 and subsequently with streptavidin-microbeads. The purified T cells (>95% purity) were labeled with 1.5 μM CFSE, washed, and then suspended in HBSS solution. 20×10⁶ or 50×10⁶ CFSE-labeled cells were transferred into the NOD and BALB/c mice. After 2 hours (for mice transferred with 50×10⁶ cells) or 48 hours (for mice transferred with 20×10⁶ cells), recipients were sacrificed, and the frequency of transferred CD4 T cells was determined in bone marrow, lymph nodes, and spleen by flow cytometry assaying for CFSE and CD4. To correct for differences in the input cell numbers among individual recipient mice, the homing index (HI) was calculated: HI=[% CFSE⁺ T cells in BM or LN]/[% CFSE⁺ CD4 T cells in spleen].

Statistics

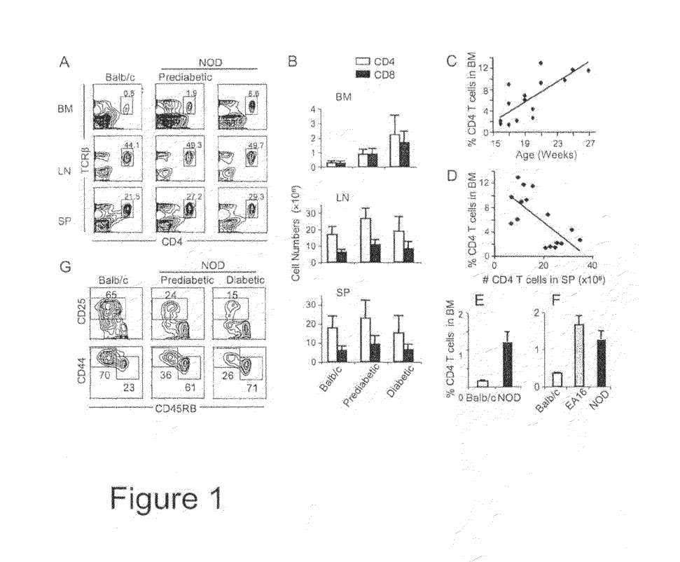

Statistical analysis for significance was done with either a two-tailed Student's t-test or a Kaplan-Meier product limit estimation. Error bars shown in FIGS. 1-12 are one standard deviation.

List of Abbreviations

CXCL12, chemokine (C-X-C motif) ligand 12; SDF-1, stromal cell-derived factor-1; CXCR4, CXC chemokine receptor 4; CFA, complete Freund's adjuvant; G-CSF, granulocyte colony-stimulating factor; HSC, hematopoietic stem cells; TID, type I diabetes.

Results

Naïve T Cells Accumulate in the Bone Marrow of NOD Mice

In the present studies of T cell homeostasis, the distribution of both naïve and memory T cells in various organs were compared between NOD mice and wild type mice. Compared to age-matched BALB/c or C57BL/6 mice, prediabetic NOD mice (15-16 week-old and no detectable urine glucose) had a significantly higher percentage of CD4+ T cells (approximately 3-fold) in the bone marrow but not in the spleen or lymph nodes (FIG. 1A). The increase was even more pronounced in diabetic NOD mice (23 week-old, urine glucose >500 mg/dl), reaching up to 15 fold of that in BALB/c mice (FIG. 1A). Correspondingly, the number of CD4 T cells in the bone marrow of prediabetic NOD mice (0.86±0.38×10⁶) was 3 times higher than that in BALB/c bone marrow (41.6±11.1×10⁶), although both bone marrows had similar numbers of cells (41.6±11.1×10⁶ versus 64.4±13.3×10⁶, p<0.09) (FIG. 1B). In diabetic NOD mice, the percentages of CD4 T cells in the bone marrow increased with age (FIG. 1C) and the increase was correlated with a decrease of CD4 T cell number in the spleen (FIG. 1D). A similar increase in the percentage and number of CD8 T cells was also observed in the bone marrow of NOD mice (FIG. 1B).

To investigate the role of inflammation or hyperglycemia in the T cell accumulation in the bone marrow of NOD mice, 4-5 week-old NOD mice were used in which insulitis (inflammation) was minimal. As shown in FIG. 1E, the percentage of CD4 T cells in the bone marrow was significantly higher in NOD mice (1.2±0.3%) than in age-matched BALB/c mice (0.2±0.03%). Furthermore, in EA16 mice, which do not typically develop diabetes because transgenic expression of an I-E molecule confers almost complete protection of NOD mice from insulitis, possibly by preventing lymphocyte infiltration into the pancreatic islets (Le Mcur et al., 1985, Nature, 316:38-42; incorporated herein by reference), the percentage of CD4 T cells in the bone marrow was significantly increased as compared to BALB/c mice and even regular NOD mice (FIG. 1F). In addition, most CD4 T cells in the bone marrow of prediabetic NOD mice were CD44^{lo}CD45RB^{hi}CD25⁻ naïve T cells (FIG. 1G). The proportion of naïve T cells increased as NOD mice became diabetic. Together, these results demonstrate that naïve T cells accumulate in the bone marrow of NOD mice.

Increased Homing Leads to T Cell Accumulation in the Bone Marrow of NOD Mice

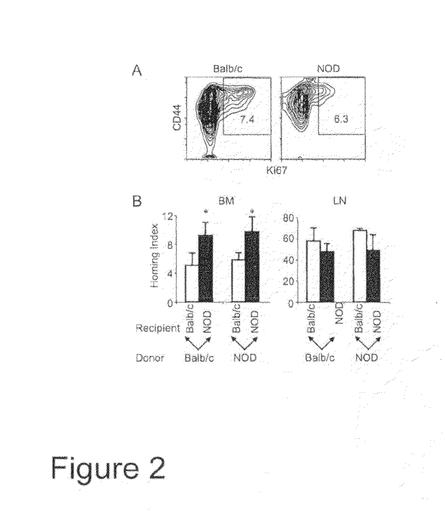

To determine if T cell accumulation in the bone marrow of NOD mice was due to proliferation of bone marrow, T cells in the bone marrow were stained for cycling marker Ki67. No significant difference in the percentages of Ki67⁺ proliferating CD4 T cells was detected in the bone marrow of prediabetic NOD mice and age-matched BALB/c mice (FIG. 2A), although a significantly higher proportion of proliferating T cells was detected in lymph nodes and spleen of NOD mice. As in BALB/c mice, proliferating CD4 T cells in the bone marrow of NOD mice were CD44^{hi} effector or memory T cells. Thus, it is unlikely that proliferation of T cells in the bone marrow of NOD mice accounts for their accumulation of naïve T cells at this site.

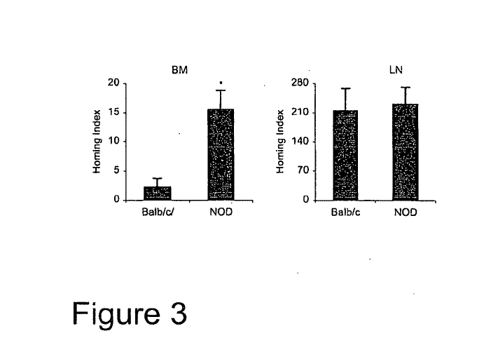

To investigate the role of recruitment in T cell accumulation in the bone marrow, total T cells were purified from NOD and BALB/c mice, labeled with CFSE, and transferred into both NOD and BALB/c mice. Distribution of CFSE-positive donor CD4 T cells in the recipients was assayed 2 hours following the transfer. No significant difference was detected in T cell distribution in the lymph nodes between BALB/c and NOD mice when either NOD or BALB/c T cells were transferred (FIG. 2B, right panel), demonstrating that T cells from BALB/c and NOD mice have equivalent motility. However, significantly more transferred BALB/c and NOD T cells were detected in the bone marrow of NOD than BALB/c mice (FIG. 2B, left panel). Forty-eight hours after the adoptive transfer, the difference between T cell distribution in the bone marrow of NOD and BALB/c recipients was even greater (FIG. 3). Without wishing to be bound by any one theory, enhanced recruitment may contribute to the accumulation of naïve T cells in the bone marrow of NOD mice. Because the enhanced recruitment is restricted to the bone marrow and independent of the source of the donor T cells, factors that mediate the preferential homing of naïve. T cells likely reside in the bone marrow of NOD mice.

Elevated CXCL12 Expression Promotes T Cell Recruitment to the Bone Marrow in NOD Mice

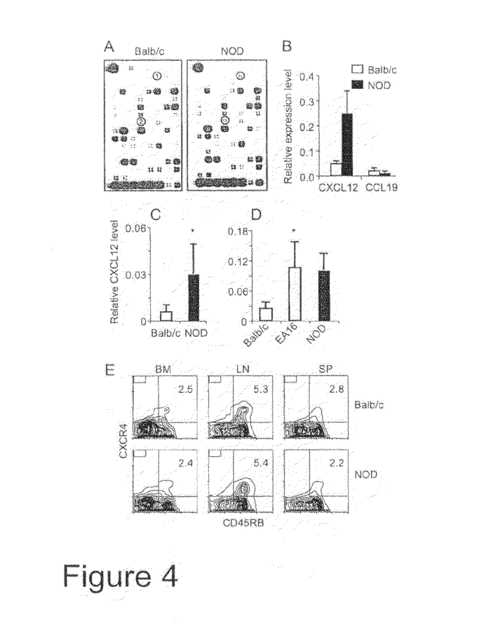

To identify chemokines that mediate the preferential homing of T cells to the bone marrow of NOD mice, as observed for the first time by the present inventors, chemokinc transcription in the bone marrow was compared between NOD mice and BALB/c mice using Mouse Chemokines & Receptors Microarrays (Superarray, Frederick, Md.). Only chemokine CXCL12 and CCL19 transcripts were elevated in the bone marrow of NOD mice as compared to that of BALB/c mice (FIG. 4A). Quantitative RT-PCR analysis showed that the CXCL12 transcript was 3-5 fold higher in the bone marrow of NOD than BALB/c mice (FIG. 4B), whereas the difference in the level of CCL19 transcript was not confirmed. Quantitative RT-PCR analysis also revealed that the CXCL12 transcript level was significantly higher in the bone marrow of 4-5 week-old NOD mice and EA16 mice than their respective age-matched controls (FIGS. 4C and 4D), correlating with increased percentages of CD4 T cells in the bone marrow of these mice (FIGS. 1E and 1F). Furthermore, majority of CD4 T cells in the spleen, lymph nodes, and bone marrow of NOD mice that expressed CXCR4 (i.e., the receptor for CXCL12) were CD45RB⁺ naïve T cells (FIG. 4E), consistent with accumulation of naïve T cells in the bone marrow. Although only a few percentages of CD4 T cells stained positive for surface CXCR4, all CD4 T cells from both NOD and C57BL/6 mice were positive for intraccllular staining of CXCR4. Because there was no difference in the percentage of CXCR4-expressing T cells between BALB/c and NOD mice, these results may suggest that it is the elevated level of CXCL12 expression that promotes homing and accumulation of the CXCR4-expressing naïve T cells in the NOD bone marrow.

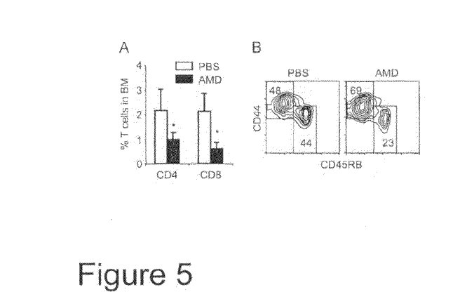

AMD3100 is a small molecule antagonist of CXCR4 (De Clercq, 2003, supra; incorporated herein by reference). The present invention encompasses the recognition that, if interaction of CXCL12 with CXCR4 were important for the presently observed naïve T cell accumulation in the bone marrow of NOD mice, treatment of NOD mice with AMD3100 would be expected to inhibit T cell accumulation in bone marrow. Thus, prediabetic NOD mice were given AMD3100 (5 mg/kg) daily for 8 days, and the distribution and phenotype of T cells in their bone marrow were assayed. AMD3100 treatment significantly reduced the proportion of both CD4 and CD8 T cells in the bone marrow of the prediabetic NOD mice as compared to prediabetic NOD mice that were given PBS (FIG. 5A). In particular, the proportion of CD4 T cells with the naïve phenotype (CD45RB^{hi} CD44^{lo}) was preferentially reduced in the AMD3100 treated NOD mice (FIG. 5B). Considering CXCL12's known function in regulating T cell migration (Moser et al., 1998, Int. Rev. Immunol., 16:323; incorporated herein by reference), the present invention encompasses the recognition that elevated CXCL12 expression may account for the accumulation of naïve T cells in the bone marrow of NOD mice.

Elevated CXCL12 Expression Promotes Recruitment/Retention of Regulatory T Cells (Tregs) and Hematopoietic Stem Cells in Bone Marrow

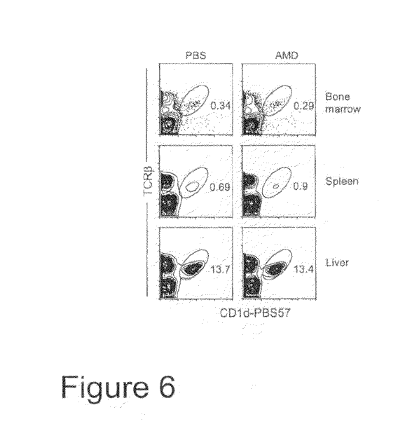

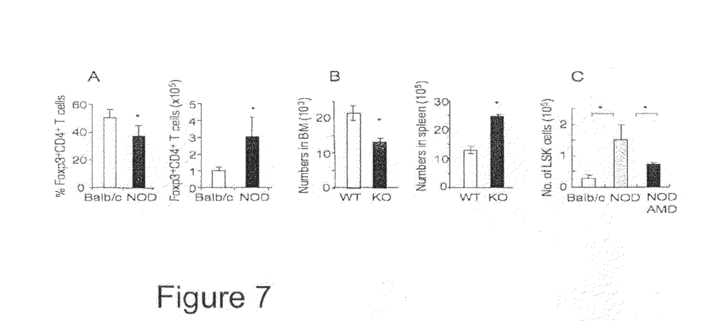

The present inventors expect that elevated CXCL12 expression would promote recruitment or retention of other cell types/subsets that express CXCR4 in the bone marrow of NOD mice. Although no significant difference in percentage or number of NKT cells, dendritic cells, or B cells was detected in the bone marrow between NOD and BALB/c mice, nor any change in distribution of these cell types in NOD mice following AMD3100 treatment (FIG. 6), the numbers of Foxp3⁺CD4⁺ Tregs were consistently higher in the bone marrow of prediabetic NOD mice than BALB/c mice (FIG. 7A). A lower percentage of CD4 T cells that are Tregs in the bone marrow of NOD mice as compared to BALB/c mice likely reflects the greater accumulation of naïve CD4 T cells than Tregs in the NOD bone marrow. To determine the involvement of CXCL12-CXCR4 interaction in regulating Treg trafficking, mice in which CXCR4 expression was specifically inactivated in T cells via Cre-mediated recombination (Zou et al., 1998, Nature 393:595-9; incorporated herein by reference) were utilized. In the absence of CXCR4, the numbers of Treg in the bone marrow was decreased significantly whereas the number of Treg in the spleen was increased as compared to littermate wild type mice (FIG. 7B). Thus, the present invention demonstrates that Treg trafficking into the bone marrow is partly regulated by CXCL12.

CXCL12 plays a role in retention of hematopoietic stem cells (HSCs) in the bone marrow of adult mice (Kucia et al., 2005, Stem Cells, 23:879-94; and Sugiyama et al., 2006, Immunity, 25:977-88; both of which are incorporated herein by reference). The number of Lin⁻Scal⁺c-Kit⁺ HSC was significantly higher in the bone marrow of prediabetic NOD mice than in age-matched BALB/c mice (FIG. 7C). Following AMD3100 treatment for 8 days, the number of HSCs in the bone marrow of NOD mice was significantly reduced.

Interference of CXCR4 Function Delays the Development of Diabetes in NOD Mice

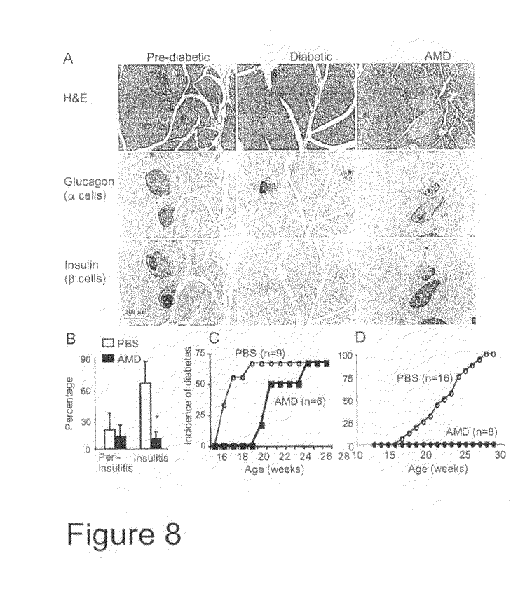

To determine the relationship between CXCL12-mediated dysregulation of T cell and stem cell trafficking and development of diabetes, NOD mice were treated with AMD3100 and progression of disease was monitored. Prediabetic NOD mice at 15 weeks of age were given AMD3100 daily for eight days and pancreata were examined by histochemical assays. As shown in FIG. 8A, prediabetic NOD mice had significantly more insulin-expressing islets than diabetic NOD mice (26.7±5.2 per section versus 15.8±9.4 per section, p<0.05). Among age-matched prediabetic NOD mice, AMD3100 treatment (daily for 8 days) significantly reduced lymphocyte infiltration into the islets (insulitis; FIGS. 8A and 8B). When prediabetic NOD mice (15-16 weeks of age) were given AMD3100 or PBS daily for three weeks, PBS-treated NOD mice rapidly developed diabetes between 17 and 20 weeks of age as indicated by glucose levels in the urine (>500 mg/dl) (FIG. 8C). In contrast, none of the AMD3100-treated mice developed the disease during the same period. However, two weeks after AMD3100 treatment was terminated, AMD3100-treated mice began to develop diabetes and the incidence reached the same level (70%) as PBS-treated mice by 26 weeks of age.

To test whether continuous AMD3100 treatment can inhibit the development of diabetes, prediabetic NOD mice (15 weeks of age) were given AMD3100 daily for 14 weeks. PBS-treated NOD mice started to show evidence of diabetes at 16 weeks of age, and by 29 weeks of age, all mice developed diabetes (FIG. 8D). In contrast, none of the AMD3100-treated mice developed the disease during the same period. Thus, the present invention encompasses the recognition that AMD3100 treatment inhibits leukocyte infiltration in the islets (insulitis) and continuous treatment delays development of overt diabetes.

Elevated CXCL12 Expression and AMD3100 Treatment Affect Disease Progression in NOD Mice Through Multiple Mechanisms

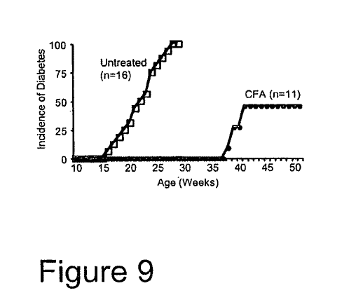

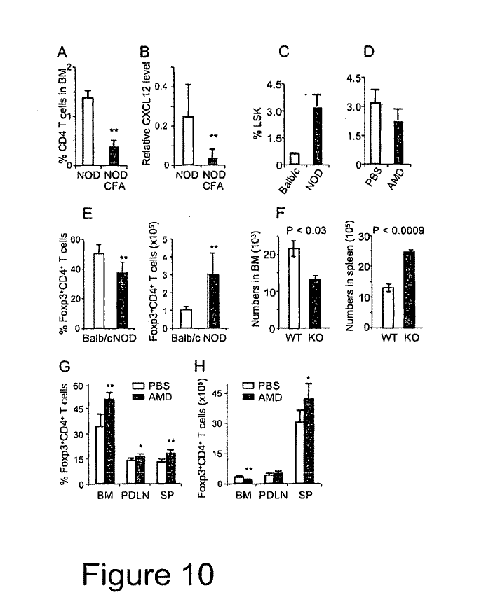

Evidence presented so far supports a model wherein elevated CXCL12 expression in the bone marrow results in dysregulated T cell trafficking, which in turn contributes to diabetes in NOD mice. Inhibition of dysregulated T cell trafficking by AMD3100 delays disease progression. To provide additional support for this model, the effect of complete Freund's adjuvant (CFA), which is different from AMD3100 but is known to prevent diabetes in NOD mice (FIG. 9), on CXCL12 expression and accumulation of T cells in the bone marrow was determined. NOD mice (11 weeks of age) were given a single CFA or PBS injection subcutaneously and, two weeks later, percentage of CD4 T cells and CXCL12 transcript level in bone marrow were determined. CFA treatment significantly reduced T cell accumulation as well as CXCL12 expression in the bone marrow of NOD mice (FIGS. 10A and 10B), supporting a role of CXCL12-mediated dysregulation of T cell trafficking in diabetes development in NOD mice.

CXCL12 is involved in mobilization of stem cells, including hematopoietic stem cells (HSC), which have been implicated in islet regeneration in NOD mice. While not wishing to be bound by any one theory, elevated CXCL12 might contribute to diabetes in NOD mice by dysregulating stem cell mobilization. Elevated CXCL12 expression in the bone marrow might be expected to retain HSC in the bone marrow. Consistent with this prediction, the percentage of Lin⁻Scal⁺c-Kit⁺ (LSK) HSC was significantly higher in NOD mice than in age-matched BALB/c mice (FIG. 10C). Furthermore, AMD3100 treatment reduced the percentage of HSC in the bone marrow of NOD mice as compared to PBS-treated NOD mice (FIG. 10D). Although the significance of the reduction is at the borderline (p=0.06), an AMD3100 treatment lasting longer than 8 days might produce more significant results.

Evidence suggests that T regulatory cells (Tregs) play a significant role in suppressing diabetes in NOD mice. To investigate whether elevated CXCL12 expression affects Treg trafficking, Treg distribution in NOD mice and the effect of AMD3100 treatment on this distribution was determined. At both 8 and 12 weeks of age, the number of Foxp3⁺CD4⁺ Tregs was consistently higher in the bone marrow of NOD mice than BALB/c mice (FIG. 10E). A lower percentage of CD4 T cells that are Tregs in the bone marrow of NOD mice as compared to BALB/c mice likely reflects the greater accumulation of naïve CD4 T cells than Tregs in the NOD bone marrow. Conditional deletion of CXCR4 only in T cells via ere-mediated recombination resulted in a decrease of Treg numbers in the bone marrow and a concomitant increase in Treg numbers in the spleen as compared to littermate wild type mice (FIG. 10F). These results suggest that Treg trafficking into the bone marrow is at least partly regulated by CXCL12.

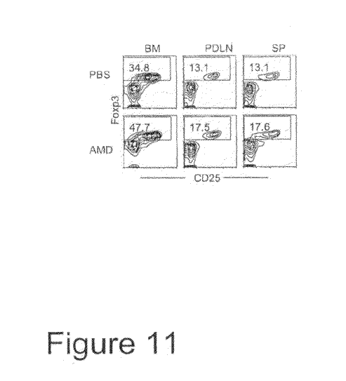

Following AMD3100 treatment for 8 days, the percentage of Foxp3⁺CD4⁺ Treg cells in the bone marrow increased while the total number of Tregs decreased (FIGS. 10G, 10H, and 11). Without wishing to be bound by any one theory, these results may reflect that the treatment promotes emigration of both naïve and Treg cells from the bone marrow, and more naïve T cells leave bone marrow than Tregs. In addition, the percentages of Tregs increased significantly in both the spleen and pancreas-draining lymph node (PDLN) following AMD3100 treatment and the number of Tregs also increased significantly in the spleen (FIGS. 10G and 10H). Because the number of Treg was relatively small in the bone marrow as compared to that in the spleen (FIG. 10H), mobilization of Tregs from the bone marrow is unlikely to be sufficient to account for the increase in Treg number in the spleen.

### Discussion

Development of type I diabetes (TID) in both humans and NOD mice is age-dependent but variable in onset, suggesting that multiple factors contribute to disease progression. Indeed, in NOD mice, studies have shown that factors that affect T cell development, function, homeostasis, and trafficking all impinge on the disease development (Anderson and Bluestone, 2005, supra; and Andre et al., 1996, supra; both of which are incorporated herein by reference). The present invention encompasses the recognition that elevated CXCL12 expression in the bone marrow of NOD mice contributes to disease progression. The present invention encompasses the recognition that this may occur by altering T cell trafficking and stem cell mobilization. The present invention encompasses the recognition of a common mechanism by which diverse processes and interventions (e.g., Tregs, lymphopenia [homeostasis], trafficking, etc.) might modulate disease progression in NOD mice.

The present invention shows that the level of CXCL12 transcript was significantly elevated in the bone marrow of NOD mice as compared to BALB/c mice (FIGS. 4A and 4B). Elevated expression is specific for CXCL12 among 33 chemokines examined. Because it was also detected in the bone marrow of 4-5 week old NOD mice and EA16 mice, elevated expression is not induced by inflammation associated with insulitis and diabetes in NOD mice. Instead, these results suggest that NOD mice are predisposed to elevated CXCL12 expression in the bone marrow, although the underlying mechanism has yet to be determined.

CXCL12 (also known as SDF-1) is a chemokine and specifically stimulates chemotaxis of cells that express CXCR4 (Nagasawa et al., 1996, Nature, 382:635; Tachibana et al., 1998, Nature, 393:591; and Zou et al., 1998, Nature, 393:595; all of which are incorporated herein by reference). Due to the surprising discoveries of the present invention, the present inventors expect that elevated CXCL12 levels could have a significant effect on recruitment and retention of CXCR4-expressing cells in the bone marrow of NOD mice. Based on analyses of young prediabetic NOD mice, diabetic NOD mice, and EA16 mice, the phenotype of CXCR4-expressing T cells, the effect of AMD3100 treatment, and direct recruitment assay, the present inventors have demonstrated for the first time that elevated CXCL12 expression results in recruitment and accumulation of naïve T cells in the bone marrow of NOD mice. First, elevated CXCL12 expression is consistently correlated with increased proportion and number of naïve T cells in the bone marrow in young (4-5 week old), prediabetic (15-16 week old) NOD mice and EA16 mice. Second, CFA inhibited CXCL12 expression and simultaneously abolished the accumulation of naïve T cells in the bone marrow of NOD mice. Third, inhibition of CXCR4 function with a small molecule antagonist prevented accumulation of naïve CD4 T cells in the bone marrow. Fourth, adoptive transferred NOD and BALB/c T cells preferentially homed to the bone marrow of NOD mice but not BALB/c mice, demonstrating a direct role of recruitment in the observed accumulation of naïve T cells. Fifth, most of CXCR4-expressing T cells are of naïve phenotype, consistent with their accumulation in the bone marrow of NOD mice. Because there are no difference in proportion of T cells that express CXCR4 between NOD and BALB/c mice and because BALB/c T cells are also preferentially recruited to the NOD bone marrow, the observed recruitment and accumulation of naïve T cells in the bone marrow of NOD mice appear to be due solely to the elevated CXCL12 expression.

In addition, the present inventors have made the surprising discovery that elevated CXCL12 expression also leads to accumulation and retention of Tregs and hematopoietic stem cells in bone marrow. A small fraction of Tregs expressed CXCR4. Deletion of CXCR4 only in T cells resulted in a significant decrease of Treg numbers in the bone marrow and a concomitant increase in the spleen, suggesting that CXCL12-CXCR4 interaction is one of the factors that regulate Treg trafficking. Consistently, the number of Tregs was significantly elevated in the bone marrow of NOD mice than BALB/c mice (FIG. 10E). Inhibition of CXCR4 by AMD3100 treatment led to reduction of Treg numbers in the bone marrow. In addition, CXCL12 is also known to regulate mobilization of stem cells, including HSC. Elevated CXCL12 expression is expected to retain HSC in the bone marrow of NOD mice. Indeed, the percentage of Lin⁻Scal⁺c-Kit⁺ HSC was significantly higher in NOD mice than BALM mice, despite the accumulation of naïve T cells in the NOD bone marrow. Inhibition of CXCR4 by AMD3100 led to mobilization of HSC out of the bone marrow, consistent with previous results. Thus, the present invention encompasses the recognition that elevated CXCL12 expression results in the recruitment and accumulation of naïve T cells, Tregs, and HSC in the bone marrow of NOD mice.

What is the relationship between elevated CXCL12 expression in bone marrow and development of diabetes in NOD mice? The present inventors have demonstrated that elevated CXCL12 expression is unlikely a consequence of inflammation or hyperglycemia associated with insulitis or diabetes in NOD mice because CXCL12 transcript was detected in the bone marrow of EA16 mice and young (4-5 week old) NOD mice. The present invention encompasses the recognition that, more likely, NOD mice are predisposed to express elevated CXCL12 in the bone marrow. Conversely, because EA16 mice do not develop diabetes despite elevated CXCL12 expression and accumulation of T cells in bone marrow, elevated CXCL12 expression alone is insufficient to initiate diabetes in the absence of autoreactive T cells. Nevertheless, the elevated CXCL12 expression is likely promote diabetes development. In humans, polymorphisms in CXCL12 have been linked to susceptibility to TID (Kared et al., 2005, supra; and Semerad et al., 2005, supra; both of which are incorporated herein by reference). Using allele-specific transcript quantification in Epstein-Barr virus-transformed lymphoblastoid cell lines, one study reported evidence that polymorphisms have a cis-acting effect on CXCL12 transcription (Kimura et al., 2005, Hum. Mol. Genet., 14:1579; incorporated herein by reference). However, whether TID patients with specific CXCL12 polymorphisms have elevated CXCL12 expression has not been reported. In NOD mice, neutralization of CXCL12 by administration of antibody suppresses insulitis and delays the onset of diabetes (Matin et al., 2002, Immunology, 107:222; incorporated herein by reference). Administration of G-CSF, a cytokine known to inhibit CXCL12 expression, also reduced insulitis and diabetes in NOD mice (Hayada et al., 2005; Kared et al., 2005, supra; both of which are incorporated herein by reference). Thus, the present invention encompasses the recognition that elevated CXCL12 expression and its associated effect on cell trafficking and mobilization is positively correlated with disease development and/or progression in NOD mice. The present inventors have further shown that complete Freund adjuvant (CFA), which is known to inhibit diabetes development (Sadelain et al., 1990, Diabetes, 39:583-9; and McInerney et al., 1991, Diabetes, 40:715-25; both of which arc incorporated herein by reference), also inhibits CXCL12 expression (Ucda et al. J. Exp. Med., 199:47-58; incorporated herein by reference) and T cell accumulation in the bone marrow of NOD mice (FIGS. 9 and 10). Finally, treatment of prediabetic NOD mice with AMD3100 abolishes accumulation of T cells and HSC in the bone marrow and simultaneously inhibits disease development (FIG. 8). However, EA16 mice do not develop diabetes despite elevated CXCL12 expression and accumulation of T cells in the bone marrow, suggesting that the elevated CXCL12 expression alone is insufficient to initiate diabetes in the absence of autoreactive T cells. Without wishing to be bound by any one theory, the elevated CXCL12 expression may contribute to type I diabetes by impinging on the rate of disease progression. Because AMD3100 is unlikely to affect CXCL12 expression in the bone marrow, contribution of the elevated CXCL12 expression in disease progression is likely to occur through its biological effect on cell trafficking and mobilization.

The present inventors encompass the recognition of potential mechanisms by which elevated CXCL12 expression promotes disease progression in NOD mice. The inventors have demonstrated that the elevated CXCL12 expression leads to homing of naïve T cells to the bone marrow. A recent study showed that diabetogenic T cells also preferentially home to the bone marrow of NOD mice (Li et al., 2007, Diabetes, 56:2251-9; incorporated herein by reference), perhaps as a result of the elevated CXCL12 expression shown here. It is possible that accumulation of T cells in the bone marrow of NOD mice might lead to lymphopenia in the peripheral lymphoid organs and homeostatic proliferation and differentiation of autoreactive T cells (King et al., 2004, Cell, 117:265-77; incorporated herein by reference; Jameson, 2002, Nat. Rev. Immunol., 2:547-56; Cho et al., 2000, J. Exp. Med., 192:549-56; and Goldrath et al., 2000, J. Exp. Med., 192:557-64; all of which are incorporated herein by reference), which in turn promotes diabetes development. It is also possible that elevated CXCL12 expression may promote disease progression in NOD mice through its effect on trafficking of autoreactive T cells, especially into islets. Studies have shown that CXCL12-CXCR4 interaction is required for recruitment of autoreactive T cells to rheumatoid arthritis synovium and the inflamed joint of collagen-induced arthritis (Matthys et al., 2001, J. Immunol., 167:4686-92; and De Klerck et al., 2005, Arthritis Res. Ther., 7:R1208-20; both of which arc incorporated herein by reference). In a virus-induced TID model, blockade of CXCL10 (IP-10) prevents diabetes development by impeding expansion of autoreactivc T cells and their migration into the pancreas (Christen et al., 2003, J. Immunol., 171:6838-45; incorporated herein by reference). The present inventors demonstrated that inhibition of CXCR4 by AMD3100 significantly reduced insulitis (FIG. 5B). Although no difference in CXCL12 expression was detected in pancreas between prediabetic NOD mice and BALB/c mice, dysregulation of lymphocyte trafficking is likely a contributing factor to the development of TID in NOD.

Elevated CXCL12 expression may promote disease progression in NOD mice through its effect on Treg trafficking, which is regulated in part by CXCL12-CXCR4 interaction. A large body of evidence suggests that Tregs play a role in suppressing autoimmunity (Sakaguchi et al., 2005, Nat. Immunol, 6:345-52; incorporated herein by reference). In NOD mice, the frequency and function of Foxp3⁺ Tregs were reported to decrease with age (Pop et al., 2005, J. Exp. Med., 201:1333-46; incorporated herein by reference). The present study found significantly more Tregs in the bone marrow of NOD mice than age-matched BALB/c mice (FIG. 8B). Following AMD3100 treatment of NOD mice, the number of Tregs in was significantly decreased in the bone marrow, whereas the number was significantly increased in the spleen (FIG. 10). However, because the number of Tregs in the bone marrow is only about one tenth of that in the spleen, Treg mobilization from the bone marrow alone cannot account for the significant increase in Treg numbers in the spleen following AMD3100 treatment. Regardless of where splenic Tregs come from, because a threshold ratio of Tregs to autoreactive T cells is important for Treg suppression of autoreactive T cells (Salomon et al., 2000, supra; and Tang et al., 2003, J. Immunol., 171:3348; both of which are incorporated herein by reference), sequestering Tregs in the bone marrow or in other organs in NOD mice may tip the balance in favor of autoreactive T cells. Consistent with this interpretation, a higher percentage of T cells proliferated in the spleen and PDLN, but not in the bone marrow of NOD than BALB/c mice. However, the extent by which elevated CXCL12 in the bone marrow promotes disease progression in NOD mice through altered Treg trafficking remains to be determined.

Elevated CXCL12 expression may promote disease progression in NOD mice through its effect on trafficking of autoreactive T cells, especially into islets. The present study found that AMD3100 treatment significantly reduced insulitis without affecting peri-insulitis in prediabetic NOD mice (FIGS. 8A and 8B), suggesting that CXCL12-CXCR4 interaction may be required for infiltration of autoreactive T cells into islets but not migration into the pancreas. These findings are consistent with previous observation that CXCL12-CXCR4 interaction is required for recruitment of autoreactive T cells to rheumatoid arthritis synovium and the inflamed joint of collagen-induced arthritis (Dc Klcrck et al., 2005, Arthritis Res. Ther., 7:R1208; and Matthys et al., 2001, J. Immunol, 167:4686; both of which are incorporated herein by reference). Without wishing to be bound by any one theory, because AMD3100 treatment did not significantly affect the percentage of T cells that proliferated in the spleen and pancreas-draining lymph nodes, the observed inhibition of insulitis may be due to inhibition of T cell infiltration into the islets. However, a significant difference in the levels of CXCL12 transcript in pancreas between NOD mice and BALB/c mice at 8, 12 and 16 weeks of age was not detected.

Elevated CXCL12 expression also leads to retention of hematopoietic stem cells (HSCs) in the bone marrow. Because bone marrow-derived stem cells appear to initiate pancreatic regeneration (Hess et al., 2003, Nat. Biotech., 21:763-70; incorporated herein by reference), it is possible that sequestering HSCs and possibly other stem/progenitor cells might contribute to disease progression in NOD mice by limiting their availability for islet regeneration. In support of this notion, systemic administration of CXCL12 ameliorates diabetes (Yano et al., 2007, Diabetes, 56:2946-57; incorporated herein by reference), perhaps partly by mobilizing HSCs from bone marrow to the periphery. The same mechanism may partly explain the effectiveness of CFA plus adoptive transfer of splenocytes, which contain a significant number of HSC and probably other progenitor cells (Ge et al., 2002, Proc. Natl. Acad. Sci., USA. 99:2989-94; incorporated herein by reference), in curing diabetic NOD mice through regeneration of islets (Chong et al., 2006, supra; Kodama et al., 2003, supra; Nishio et al., 2006, supra; and Suri et al., 2006, supra; all of which are incorporated herein by reference). Although further investigations are required to determine if mobilization of progenitor cells required for islet regeneration is impaired by elevated CXCL12 expression in NOD mice, the present findings already suggest a possible common mechanism by which diverse cell types and factors (e.g., Treg, lymphopenia, lymphocyte trafficking, etc.) may contribute to type I diabetes via cell trafficking and/or mobilization. As altered cell trafficking and/or mobilization are likely to vary among individual mice, it provides for an explanation of the variable onset of diabetes in NOD mice.

AMD3100 was originally developed to treat HIV infection through its antagonism of the CXCR4, a co-receptor for the virus (De Clercq, 2003, supra; incorporated herein by reference). Currently, it is being developed as an agent to mobilize HSCs from bone marrow to peripheral blood for transplantation of stem cells in patients with non-Hodgkin's lymphoma or multiple myeloma (Devine et al., 2004, J. Clin. Oncol., 22:1095; incorporated herein by reference). As the inventors have shown here, administration of AMD3100 to prediabctic NOD mice significantly delays insulitis and the onset of diabetes. In contrast, a recent study reported that AMD3100 treatment promotes diabetes development in a model where diabetes was induced by adoptive transfer of splenocytes from female NOD mice into sublethally irradiated male NOD mice (Aboumrad et al., 2007, Clin. Exp. Immunol., 148:432-9; incorporated herein by reference). In the transfer model, transferred T cells undergo homeostatic proliferation and acquire effector functions (Jameson, 2002, Nat. Rev. Immunol., 2:547-56; Cho et al., 2000, J. Exp. Med., 192:549-56; and Goldrath et al., 2000, J. Exp. Med., 192:557-64; all of which are incorporated herein by reference). Irradiation of recipient mice also introduces additional factors and processes which are not active in non-irradiated prediabetic mice. Thus, disease induction mechanisms in the transfer model could be significantly different from those in un-manipulated female NOD mice. Although the precise mechanism underlying the discrepancy between the two studies has yet to be determined, the discoveries made by the present inventors provide a basis for further exploring the use of AMD3100 and/or any other agent that negatively affects the interaction between CXCL12 and CXCR4 to prevent and/or treat type I diabetes and/or any other autoimmune disease in patients with elevated CXCL12 or CXCR4 expression.

Thus, the present invention encompasses the recognition that elevated CXCL12 expression in bone marrow of NOD mice likely promotes development of TID by altering T cell and hematopoietic stem cell trafficking. The discoveries made by the present inventors suggest, for the first time, the possibility of preventing and/or treating TID in humans by modulating either the expression or function of CXCL12 and CXCR4. The present invention encompasses the recognition that studies performed in NOD mice may be generally applicable to treatment in humans. The present invention encompasses the recognition that TED in humans may be treated, prevented, ameliorated, delayed, and/or improved by modulating the interaction between CXCL12 and CXCR4, by modulating the expression of CXCL12 and/or CXCR4, and/or by modulating the function of CXCL12 and/or CXCR4.

### Equivalents and Scope

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any clement(s) can be removed from the group. It should it be understood that, in general, where the invention, or aspects of the invention, is/arc referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any CXCL12 and/or CXCR4 antagonist; any method of assaying CXCL12 or CXCR4 levels in particular tissues, such as the bone marrow or bloodstream; any tissue; any method of treatment and/or diagnosis; any therapeutic application; etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. A method comprising steps of:
providing a sample from blood or bone marrow of a patient who is suffering from or susceptible to an autoimmune disorder;
analyzing levels of CXCL12 or CXCR4 in the sample;
determining, based on the analysis, whether the patient has elevated levels of CXCL12 or CXCR4 as compared with a reference sample; and
classifying the patient as likely to respond to treatment with a CXCL12 or CXCR4 antagonist if the determination reveals elevated levels of CXCL12 or CXCR4, or classifying the patient as unlikely to respond to treatment with a CXCL12 or CXCR4 antagonist if the determination does not reveal elevated levels of CXCL12 or CXCR4.

2. The method of claim 1, further comprising a step of administering a CXCL12 or CXCR4 antagonist to a patient displaying elevated levels of CXCL12 or CXCR4 in the sample.

3. A method comprising steps of:
providing a sample from bone marrow of a patient who is suffering from or susceptible to an autoimmune disorder;
analyzing levels of T cells or stem cells in the sample;
determining, based on the analysis, whether the patient has elevated levels of T cells or stem cells as compared with a reference sample; and
classifying the patient as likely to respond to treatment with a CXCL12 or CXCR4 antagonist if the determination reveals elevated levels of T cells or stem cells, or classifying the patient as unlikely to respond to treatment with a CXCL12 or CXCR4 antagonist if the determination does not reveal elevated levels of T cells or stem cells.

4. The method of claim 3, further comprising a step of administering a CXCL12 or CXCR4 antagonist to a patient displaying elevated levels of T cells or stem cells in the sample.

5. The method of claim 1, wherein the CXCL12 or CXCR4 antagonist is administered in combination with at least one additional therapeutic agent.

6. The method of claim 1, wherein the CXCL12 or CXCR4 antagonist inhibits the interaction of CXCL12 and CXCR4

7. The method of claim 1, wherein the CXCL12 or CXCR4 antagonist is a small molecule.

8. The method of claim 1, wherein the CXCL12 or CXCR4 antagonist is AMD3100.

9. The method of claim 1, wherein the CXCL12 or CXCR4 antagonist is an antibody that recognizes CXCL12 or CXCR4.

10. The method of claim 1, wherein the step of analyzing levels of CXCL12 or CXCR4 in the sample is performed by directly measuring the levels of CXCL12 or CXCR4 in the sample.

11. The method of claim 1, wherein the step of analyzing levels of CXCL12 or CXCR4 in the sample is performed indirectly and is not performed by directly measuring the levels of CXCL12 or CXCR4 in the sample.

12. The method of claim 1, wherein the reference sample is obtained from a patient who does not display symptoms of or has not been diagnosed with an autoimmune disorder.

13. The method of claim 1, wherein the autoimmune disease is type I diabetes.

14. A method comprising steps of:
providing a patient suffering from or susceptible to an autoimmune disorder; and
administering a CXCL12 or CXCR4 antagonist to the patient in an amount sufficient to treat, alleviate, ameliorate, relieve, delay onset of, inhibit progression of, reduce severity of, or reduce incidence of one or more symptoms or features of the autoimmune disorder.

15-16. (canceled)

17. The method of claim 1, wherein the autoimmune disease is type I diabetes.

18. The method of claim 1, wherein the CXCL12 or CXCR4 antagonist is administered in combination with at least one additional therapeutic agent.

19. (canceled)

20. A kit, comprising:
a library of candidate substances;
a CXCL12 and/or CXCR4 antagonist that may serve as a positive control;
a substance that may serve as a negative control.

21-25. (canceled)
